SEQUENCE LISTING (18 sequences)

SEQ ID NO: 1 - protein, 275 aa, Citrullus lanatus
MVNNALLEALNVRVLGTGDRSLVLAHGFGTDQSAWQLIYPSFTPYYRVILYDLVCAGSVNPDFFDFSRYTTLDAFVDDLISILDSLHVHRCAFVGHSVSAMVGHSVSAMVGILASIRRPELFSKLILIGASPRFLNDGDYHGGFEQSEIDRVFAAMKANYQSWVNGFAPLAVGADVPAAVQEFSRTLFNMRPDISLFVSKVIFSSDLRGVLGLVKVPCCIIQTAQDVSVPASVAIYLRDHLGGRNTVEMLDTEGHLPHLSAPQLLVRKLRRALSR

SEQ ID NO: 2 - protein, 267 aa, Citrullus lanatus
MVNNALLEALNVRVLGTGDRSLVLAHGFGTDQSAWQLIYPSFTPYYRVILYDLVCAGSVNPDFFDFSRYTTLDAFVDDLISILDSLHVHRCAFVGHSVSAMVGILASIRRPELFSKLILIGASPRFLNDGDYHGGFEQSEIDRVFAAMKANYQSWVNGFAPLAVGADVPAAVQEFSRTLFNMRPDISLFVSKVIFSSDLRGVLGLVKVPCCIIQTAQDVSVPASVAIYLRDHLGGRNTVEMLDTEGHLPHLSAPQLLVRKLRRALSR

SEQ ID NO: 3 - DNA, 828 bp, Citrullus lanatus
atggttaacaacgcccttcttgaagcccttaatgtccgtgtcctcggcaccggcgaccgttctctggtcctggcccatggcttcggcaccgaccagtccgcttggcaactcatttacccttcctttactccttactaccgcgtcatcctttacgaccttgtctgcgccggtagcgtcaaccccgacttcttcgatttctcccgctacaccactctcgacgccttcgtcgacgatctcatctccatcctagactctctccacgtccaccgctgcgcctttgtcggccactccgtctccgccatggtcggccactccgtctccgccatggtcggcatcctcgcctccatccgccgtcccgaactcttctctaagctcatcttaatcggcgcctccccaaggttcctcaacgacggcgactaccacggtgggttcgaacagagcgagattgacagggtcttcgctgcaatgaaggctaattaccaatcctgggtcaacggctttgcccctcttgctgtcggtgccgatgttcccgctgccgttcaggaattcagccggactctcttcaatatgagacccgacatttccctcttcgtctctaaggtcatcttcagcagcgatctccggggagtcctcggtctcgtcaaagtcccctgttgcataattcaaaccgcccaagacgtctctgttccggcctccgtcgctatctacctccgagaccacctcggcggccggaacaccgtggagatgctcgacaccgaaggccacctaccccatctgagtgcccctcagctactcgtacggaaactccgccgtgctctttcccggtga

SEQ ID NO: 4 - DNA, 804 bp, Citrullus lanatus
atggttaacaacgcccttcttgaagcccttaatgtccgtgtcctcggcaccggcgaccgttctctggtcctggcccatggcttcggcaccgaccagtccgcttggcaactcatttacccttcctttactccttactaccgcgtcatcctttacgaccttgtctgcgccggtagcgtcaaccccgacttcttcgatttctcccgctacaccactctcgacgccttcgtcgacgatctcatctccatcctagactctctccacgtccaccgctgcgcctttgtcggccactccgtctccgccatggtcggcatcctcgcctccatccgccgtcccgaactcttctctaagctcatcttaatcggcgcctccccaaggttcctcaacgacggcgactaccacggtgggttcgaacagagcgagattgacagggtcttcgctgcaatgaaggctaattaccaatcctgggtcaacggctttgcccctcttgctgtcggtgccgatgttcccgctgccgttcaggaattcagccggactctcttcaatatgagacccgacatttccctcttcgtctctaaggtcatcttcagcagcgatctccggggagtcctcggtctcgtcaaagtcccctgttgcataattcaaaccgcccaagacgtctctgttccggcctccgtcgctatctacctccgagaccacctcggcggccggaacaccgtggagatgctcgacaccgaaggccacctaccccatctgagtgcccctcagctactcgtacggaaactccgccgtgctctttcccggtga

SEQ ID NO: 5 - DNA, 917 bp, Citrullus lanatus
atggttaacaacgcccttcttgaagcccttaatgtccgtgtcctcggcaccggcgaccgttctctggtcctggcccatggcttcggcaccgaccagtccgcttggcaactcatttacccttcctttactccttactaccgcgtcatcctttacgaccttgtctgcgccggtagcgtcaaccccgacttcttcgatttctcccgctacaccactctcgacgccttcgtcgacgatctcatctccatcctagactctctccacgtccaccgctgcgcctttgtcggccactccgtctccgccatggtcggccactccgtctccgccatggtcggcatcctcgcctccatccgccgtcccgaactcttctctaagctcatcttaatcggcgcctccccaaggtcctttccacttccacactctgtttttctaactactctgtttttttcccctgtttttataaaattctttttatttttatttttttcaggttcctcaacgacggcgactaccacggtgggttcgaacagagcgagattgacagggtcttcgctgcaatgaaggctaattaccaatcctgggtcaacggctttgcccctcttgctgtcggtgccgatgttcccgctgccgttcaggaattcagccggactctcttcaatatgagacccgacatttccctcttcgtctctaaggtcatcttcagcagcgatctccggggagtcctcggtctcgtcaaagtcccctgttgcataattcaaaccgcccaagacgtctctgttccggcctccgtcgctatctacctccgagaccacctcggcggccggaacaccgtggagatgctcgacaccgaaggccacctaccccatctgagtgcccctcagctactcgtacggaaactccgccgtgctctttcccggtga

SEQ ID NO: 6 - DNA, 893 bp, Citrullus lanatus
atggttaacaacgcccttcttgaagcccttaatgtccgtgtcctcggcaccggcgaccgttctctggtcctggcccatggcttcggcaccgaccagtccgcttggcaactcatttacccttcctttactccttactaccgcgtcatcctttacgaccttgtctgcgccggtagcgtcaaccccgacttcttcgatttctcccgctacaccactctcgacgccttcgtcgacgatctcatctccatcctagactctctccacgtccaccgctgcgcctttgtcggccactccgtctccgccatggtcggcatcctcgcctccatccgccgtcccgaactcttctctaagctcatcttaatcggcgcctccccaaggtcctttccacttccacactctgtttttctaactactctgtttttttcccctgtttttataaaattctttttatttttatttttttcaggttcctcaacgacggcgactaccacggtgggttcgaacagagcgagattgacagggtcttcgctgcaatgaaggctaattaccaatcctgggtcaacggctttgcccctcttgctgtcggtgccgatgttcccgctgccgttcaggaattcagccggactctcttcaatatgagacccgacatttccctcttcgtctctaaggtcatcttcagcagcgatctccggggagtcctcggtctcgtcaaagtcccctgttgcataattcaaaccgcccaagacgtctctgttccggcctccgtcgctatctacctccgagaccacctcggcggccggaacaccgtggagatgctcgacaccgaaggccacctaccccatctgagtgcccctcagctactcgtacggaaactccgccgtgctctttcccggtga

SEQ ID NO: 7 - protein, 267 aa, Arabidopsis thaliana
MSQHNILEALNVRVVGTGDRILFLAHGFGTDQSAWHLILPYFTQNYRVVLYDLVCAGSVNPDYFDFNRYTTLDPYVDDLLNIVDSLGIQNCAYVGHSVSAMIGIIASIRRPELFSKLILIGFSPRFLNDEDYHGGFEEGEIEKVFSAMEANYEAWVHGFAPLAVGADVPAAVREFSRTLFNMRPDISLFVSRTVFNSDLRGVLGLVRVPTCVIQTAKDVSVPASVAEYLRSHLGGDTTVETLKTEGHLPQLSAPAQLAQFLRRALPR

SEQ ID NO: 8 - protein, 267 aa, Cucumis sativus
MVNNALLEALNVRVLGTGDRFLVLAHGFGTDQSAWQLVYPSFTPYYRVILYDLVCAGSVNPDFFDFSRYTTLDAFVDDLISILDSLHVHRCAFVGHSVSAMVGILASIRRPELFSKLILIGASPRFLNDGDYHGGFEQNEIDRVFAAMKANYQSWVNGFAPLAVGADVPAAVQEFSRTLFNMRPDISLFVSKVIFSSDLRGVLGLVKVPCCIIQTAQDVSVPTSVAIYLRDHLGGRNTIEMLDTEGHLPHLSAPQLLVRKLRRALSR

SEQ ID NO: 9 - protein, 267 aa, Cucumis melo
MVNNALLEALNVRVLGTGDRFLVLAHGFGTDQSAWQLVYPSFTPYYRVILYDLVCAGSVNPDFFDFSRYTTLDAFVDDLISILDSLHVHRCAFVGHSVSAMVGILASIRRPELFSKLILIGASPRFLNDGDYHGGFEQSEIDRVFAAMKANYQSWVNGFAPLAVGADVPAAVQEFSRTLFNMRPDISLFVSKVIFSSDLRGVLGLVKVPCCIIQTAQDVSVPTSVAIYLRDHLGGRNTIEMLDTEGHLPHLSAPQLLVRKLRRALSR

SEQ ID NO: 10 - DNA, 18 bp, artificial
  Fam primer
gagacggagtggccgacc

SEQ ID NO: 11 - DNA, 19 bp, artificial
  Vic primer
ggagacggagtggccgaca

SEQ ID NO: 12 - DNA, 20 bp, artificial
  Common primer
cacgtccaccgctgcgcctt

SEQ ID NO: 13 - DNA, 121 bp, artificial
  DNA sequence for KASP assay with deletion
gagttcgggacggcggatggaggcgaggatgccgaccatggcggagacggagtggccgacaaaggcgcagcggtggacgtggagagagtckaggatggagatgagatcgtcracgaaggcg

SEQ ID NO: 14 - DNA, 145 bp, artificial
  DNA sequence for KASP assay with insertion
gagttcgggacggcggatggaggcgaggatgccgaccatggcggagacggagtggccgaccatggcggagacggagtggccgacaaaggcgcagcggtggacgtggagagagtckaggatggagatgagatcgtcracgaaggcg

SEQ ID NO: 15 - DNA, 885 bp, Cucumis sativus
atggttaacaacgcccttcttgaagcccttaatgtccgcgtccttggcaccggcgatcgcttcctcgtcttagcccatggcttcggcaccgaccaatccgcttggcaactcgtttacccttccttcactccctactaccgtgtcatactctacgaccttgtctgcgccggcagcgtcaaccccgacttcttcgatttctcccgttacaccactctcgacgccttcgtcgacgacctcatctccatcctcgactccctccacgtccaccgctgcgccttcgtcggccactccgtctccgccatggtcggcatcctcgcctccatccgccgtcccgaactcttctccaaactcatcttaatcggcgcatccccaaggtcgtcctttttcatttctaccctctactactctgtgtttccccctgttttcttctaaaattctcttctcatttcttttaggtttctcaacgacggcgactaccatggtgggttcgaacagaatgagatagacagggttttcgctgcaatgaaggctaattaccaatcttgggtcaacgggtttgcccctctcgctgtcggtgccgatgttcccgctgccgtacaggaattcagccgaactctcttcaatatgagacccgacatttccctctttgtttctaaggttatcttcagcagcgatctccggggagtgctcggtctcgtcaaagtcccctgctgtataattcaaaccgcccaagacgtgtctgttccgacctctgtcgccatctacctgagagaccacctcgggggccggaacaccattgagatgctggacacggaagggcacctaccccatctgagtgcccctcagcttctcgttcggaaacttcgccgtgctctttctcggtga

SEQ ID NO: 16 - DNA, 880 bp, Cucumis melo
atggttaacaacgcccttcttgaagcccttaatgtccgtgtccttggcaccggcgaccgcttcctcgtcttagcccatggcttcggcaccgaccaatccgcttggcaactcgtttacccttccttcactccttactaccgtgtcatactctacgaccttgtctgcgccggcagcgtcaaccccgacttcttcgatttctctcgttacaccactctcgacgccttcgtcgacgacctcatctccatcctcgactccctccacgtccaccgctgcgccttcgtcggccactccgtctccgccatggtcggcatccttgcctccatccgccgccccgaactcttctccaaactcatcttaatcggcgcatccccaaggtgctttccgtttctaccctattctactctgttttccccctgttttcttctaaaattctcttctcatttcttttaggtttctcaacgacggcgactaccatggtgggttcgaacagagtgagattgacagggttttcgctgcaatgaaagctaattaccaatcttgggtcaacggctttgcccctctcgctgtcggtgccgatgttcccgctgccgtacaggaattcagccggactctcttcaatatgagacccgacatttccctctttgtttctaaagttatcttcagcagcgatctccggggagtcctcggtctcgtcaaagtcccctgttgtataattcaaaccgcccaagacgtctctgttccgacctccgtcgccatctacctcagagatcacctcggcggccggaacaccattgagatgctcgacaccgaaggccacctacctcatctcagtgcccctcagctactcgtgcggaaactccgccgtgctctttctcggtga

SEQ ID NO: 17 - DNA, 804 bp, Cucumis sativus
atggttaacaacgcccttcttgaagcccttaatgtccgcgtccttggcaccggcgatcgcttcctcgtcttagcccatggcttcggcaccgaccaatccgcttggcaactcgtttacccttccttcactccctactaccgtgtcatactctacgaccttgtctgcgccggcagcgtcaaccccgacttcttcgatttctcccgttacaccactctcgacgccttcgtcgacgacctcatctccatcctcgactccctccacgtccaccgctgcgccttcgtcggccactccgtctccgccatggtcggcatcctcgcctccatccgccgtcccgaactcttctccaaactcatcttaatcggcgcatccccaaggtttctcaacgacggcgactaccatggtgggttcgaacagaatgagatagacagggttttcgctgcaatgaaggctaattaccaatcttgggtcaacgggtttgcccctctcgctgtcggtgccgatgttcccgctgccgtacaggaattcagccgaactctcttcaatatgagacccgacatttccctctttgtttctaaggttatcttcagcagcgatctccggggagtgctcggtctcgtcaaagtcccctgctgtataattcaaaccgcccaagacgtgtctgttccgacctctgtcgccatctacctgagagaccacctcgggggccggaacaccattgagatgctggacacggaagggcacctaccccatctgagtgcccctcagcttctcgttcggaaacttcgccgtgctctttctcggtga

SEQ ID NO: 18 - DNA, 804 bp, Cucumis melo
atggttaacaacgcccttcttgaagcccttaatgtccgtgtccttggcaccggcgaccgcttcctcgtcttagcccatggcttcggcaccgaccaatccgcttggcaactcgtttacccttccttcactccttactaccgtgtcatactctacgaccttgtctgcgccggcagcgtcaaccccgacttcttcgatttctctcgttacaccactctcgacgccttcgtcgacgacctcatctccatcctcgactccctccacgtccaccgctgcgccttcgtcggccactccgtctccgccatggtcggcatccttgcctccatccgccgccccgaactcttctccaaactcatcttaatcggcgcatccccaaggtttctcaacgacggcgactaccatggtgggttcgaacagagtgagattgacagggttttcgctgcaatgaaagctaattaccaatcttgggtcaacggctttgcccctctcgctgtcggtgccgatgttcccgctgccgtacaggaattcagccggactctcttcaatatgagacccgacatttccctctttgtttctaaagttatcttcagcagcgatctccggggagtcctcggtctcgtcaaagtcccctgttgtataattcaaaccgcccaagacgtctctgttccgacctccgtcgccatctacctcagagatcacctcggcggccggaacaccattgagatgctcgacaccgaaggccacctacctcatctcagtgcccctcagctactcgtgcggaaactccgccgtgctctttctcggtga

(19) United States
(12) Patent Application Publication
(10) Pub. No.: US 2025/0280777 A1
(43) Pub. Date: Sep. 11, 2025

(54) METHODS FOR SELECTING WATERMELON PLANTS AND PLANT PARTS COMPRISING A MODIFIED DWARF14 GENE

(71) Applicant: NUNHEMS B.V., Nunhem (NL)

(72) Inventors: Jayasri ALLUVADA, Davis, CA (US); Mona MAZAHERI, Davis, CA (US); Elena CHIAPPARINO, Sant Agata Bolognese (IT)

(21) Appl. No.: 18/575,550

(22) PCT Filed: Jun. 28, 2022
(86) PCT No.: PCT/EP2022/067731

§ 371 Date: Dec. 29, 2023

Related U.S. Application Data
(60) Provisional application No. 63/217,071, filed on Jun. 30, 2021.

(30) Foreign Application Priority Data
Sep. 2, 2021 (EP) 21194565.4

(51) Int. Cl. A01H 1/00; A01H 1/04; A01H 5/08; A01H 6/34; C07K 14/415; C12Q 1/6895

(52) U.S. Cl. CPC A01H 1/121; A01H 1/04; A01H 5/08; A01H 6/342; C07K 14/415; C12Q 1/6895; C12Q 2600/156

(57) ABSTRACT

The present invention is directed to a genotyping method for a gene named DWARF 14 in watermelon, cucumber or melon, which, when mutated, confers an increased secondary branching phenotype. Also plants comprising modifications in the DWARF 14 gene are provided herein.

### CROSS-REFERENCE TO RELATED APPLICATIONS

This application is a National Stage Entry of PCT/EP2022/067731, filed Jun. 28, 2022, which claims priority to European Patent Application No 21194565.4, filed Sep. 2, 2021, and U.S. Provisional Application No. 63/217,071, filed Jun. 30, 2021, the disclosures of each of which are hereby incorporated by reference in their entireties.

### FIELD

The present invention is directed to the identification of a modified (or mutant) gene in watermelon and to methods for generating and/or selecting plants and plant parts comprising a modified (or mutant) allele of the gene or the wild type allele of the gene. The gene is referred to as DW4RF14 or ClDWARF14 or ClD14, as the wild type gene is assumed to be an ortholog of the Arabidopsis thaliana AtDWARF14 (AtD14) gene. In normal watermelon plants the wild type ClD14 gene is found on chromosome 8 and encodes a ClD14 protein of 267 amino acids. A modified allele of the gene was found in multibranching watermelon plants, which contained a duplication of 8 amino acids and thus a protein of 275 amino acids, see FIG. 1. Plants homozygous for this modified allele of the ClD14 gene have a multibranching phenotype, with an average number of secondary branches being equal to or above 45 secondary branches. In contrast watermelon plants heterozygous for the modified allele or homozygous for the wild type allele have a normal branching phenotype, with an average number of secondary branches being significantly below 45, such as around below 30 secondary branches, e.g. around 20 secondary branches on average.

Other Cucurbitaceae, such as melon and cucumber, also contain genes which encode D14 proteins which have a high sequence identity to the watermelon ClD14 protein. These are referred to as e.g. CmD14 (Cucumis melo) or CsD14 (Cucumis sativus) genes and proteins. The watermelon, cucumber and melon genes and proteins may herein also be referred to just as D14 gene, D14 allele or D14 protein.

It was further, surprisingly, found that the mutant allele, which comprises the 8 amino acid duplication, actually encodes a non-functional ClD14 protein. This was unexpected, as the D14 protein is a complex protein, which interacts with various other proteins and the 8 amino acid duplication was not expected to completely abolish protein function. When screening a watermelon TILLING population, a mutant allele which encoded a truncated, non-functional protein (that lacked 113 amino acids of the 267 amino acids) surprisingly resulted in the same multibranching phenotype as the mutant allele comprising the 8 amino acid duplication. Both mutant alleles resulted in an average number of secondary branches which was about 240%, relative to the average number of secondary branches in the wild type plant (set to 100% secondary branching). This strong phenotype, caused by the non-functional protein, is referred herein as “strong multibranching” or “full multibranching”. In addition, the finding allows the generation of mutant alleles which do not result in “full multibranching”, but “intermediate multibranching”, whereby the ClD14 protein has reduced function, but not a loss of function.

Thus, in one aspect, the invention relates to watermelon plants comprising a mutant allele of ClD14 gene, which results in a non-functional ClD14 protein and full multibranching (when the mutant allele is in homozygous form), or comprising a mutant allele of the ClD14 gene, which results in a reduced function ClD14 protein and intermediate multibranching (when the mutant allele is in homozygous form). The watermelon plant comprising the mutant allele which encodes the non-functional protein of SEQ ID NO: 1 (ClD14ins) is in one aspect not encompassed.

The invention is in another aspect directed at methods for determining whether a Cucurbitaceae plant, especially a watermelon, melon or cucumber plant or plant part comprises a wild type allele of the D14 gene and/or a mutant allele of a D14 gene. The wild type allele of a D14 gene encodes a watermelon D14 protein of SEQ ID NO: 2 (or a protein comprising at least 95% sequence identity to SEQ ID NO: 2), a cucumber D14 protein of SEQ ID NO: 8 (or a protein comprising at least 95% sequence identity to SEQ ID NO: 8), or a melon protein of SEQ ID NO: 9 (or a protein comprising at least 95% sequence identity to SEQ ID NO: 9). In one aspect the mutant allele is an allele encoding a duplication of amino acids 94 to 101 of SEQ ID NO: 2 (watermelon), SEQ ID NO: 8 (cucumber) or SEQ ID NO: 9 (melon). In another aspect the mutant allele is an allele which encodes a protein comprising one or more amino acids inserted, duplicated, replaced or deleted compared to the wild type protein of SEQ ID NO: 2 (watermelon), SEQ ID NO: 9 (melon) or SEQ ID NO: 8 (cucumber) and is a reduced-function protein, leading to intermediate multibranching when the allele is in homozygous form, or is a non-functional protein, leading to full multibranching when the allele is in homozygous form.

Also a method for detecting a wild type allele or a mutant allele of a D14 gene is provided, whereby either a primer pair or an oligonucleotide probe is used to amplify or detect a D14 allele in a genomic DNA of watermelon, melon or cucumber. Oligonucleotide primers or probes comprise at least 10, 11, 12, 13, 14, 15, 16, 17, 18, 19, 20 or more consecutive nucleotides of SEQ ID NO: 5 or 6 (or the complement DNA strand of either of these), or of SEQ ID NO: 15 or 16 (or the complement DNA strand of either of these). In particular a pair of primers is provided, at least one forward primer and one reverse primer, which hybridize to and amplify part of the genomic D14 allele in a PCR reaction.

In another aspect methods for generating and/or selecting a Cucurbitaceae plant, especially a watermelon, melon or cucumber plant or plant part comprises a mutant allele of a D14 gene. In one aspect the mutant allele is an allele encoding a duplication of amino acids 94 to 101 of SEQ ID NO: 2 (watermelon), SEQ ID NO: 8 (cucumber) or SEQ ID NO: 9 (melon). In another aspect the mutant allele is an allele which encodes a protein comprising one or more amino acids inserted, duplicated, replaced or deleted compared to the wild type protein of SEQ ID NO: 2 (watermelon), SEQ ID NO: 9 (melon) or SEQ ID NO: 8 (cucumber) and is a reduced-function protein, leading to intermediate multibranching when the allele is in homozygous form, or is a non-functional protein, leading to full multibranching when the allele is in homozygous form.

In one aspect also watermelon, cucumber or melon plants and plant parts are provided comprising a mutant allele of a D14 gene. In one aspect the mutant allele encodes a protein comprising a duplication of amino acids 94 to 101 of SEQ ID NO: 2 (watermelon), SEQ ID NO: 8 (cucumber) or SEQ ID NO: 9 (melon). In another aspect the mutant allele is an allele which encodes a protein comprising one or more amino acids inserted, duplicated, replaced or deleted compared to the wild type protein of SEQ ID NO: 2 (watermelon), SEQ ID NO: 9 (melon) or SEQ ID NO: 8 (cucumber) and is a reduced-function protein, leading to intermediate multibranching when the allele is in homozygous form, or is a non-functional protein, leading to full multibranching when the allele is in homozygous form.

In one aspect a watermelon plant is provided which is heterozygous for a mutant allele of a ClD14 gene. In one aspect the mutant allele encodes a protein comprising a duplication of amino acids 94 to 101 of SEQ ID NO: 2 (watermelon). In another aspect the mutant allele is an allele which encodes a protein comprising one or more amino acids inserted, duplicated, replaced or deleted compared to the wild type protein of SEQ ID NO: 2 (watermelon), SEQ ID NO: 9 (melon) or SEQ ID NO: 8 (cucumber) and is a reduced-function protein, leading to intermediate multibranching when the allele is in homozygous form, or is a non-functional protein, leading to full multibranching when the allele is in homozygous form.

### BACKGROUND

U.S. Pat. No. 7,314,9791B2 describes a recessive allele referred to as HMBN allele, which in homozygous form increases secondary branching and decreases average fruit weight to 0.87 kg.

Also WO2006/060425 describes the recessive allele referred to as HMBN allele. On page 15 [0090], the HMBN allele is described as ‘an unexpected mutant allele that arose from a watermelon breeding project’.

Patent application US2020093086 describes watermelon plants producing small fruits of less than 0.9 kg due to the combination of the HMBN allele in homozygous form and the mutant ts-gene allele on chromosome 2.

The gene and gene location of the HMBN allele is so far unknown. Consequently, also the function of the gene is unknown.

Herein the gene for the HMBN allele has been identified and has been found to encode a ClD14 protein comprising a duplication of amino acids 94 to 101 of SEQ ID NO: 2 (the wild type ClD14 protein). This mutant protein is shown in SEQ ID NO: 1 and is also referred to as ClD14ins (for ‘insertion’) herein.

The wild type protein ClD14 (SEQ ID NO: 2) is thought to be an orthologue of the Arabidopsis DWARF14 protein. AtDWARF14 is a protein, which has been shown to have a dual function in strigolactone signaling and strigolactone hydrolysis in Arabidopsis, as mutants were made which affected either of these functions, see Seto et al. (2019, Nature Communications 10:191, Strigolactone perception and deactivation by a hydrolase receptor DWARF14). In this publication the authors describe in FIG. 5 a model of the involvement of the Arabidopsis DWARF14 (AtD14) protein in the strigolactone signaling pathway and hydrolysis. A bioactive Strigolactone molecule is perceived by the AtD14 protein and induces a conformational change of the AtD14 protein, resulting in the formation of a protein complex with other signaling proteins (such as D53). Signal transduction leads to e.g. the inhibition of branching. After signaling, the AtD14 protein changes back to its original conformation and hydrolyzes the strigolactone molecule. AtD14 is, therefore, involved in both signal transduction to e.g. inhibit branching and in the homeostasis of strigolactone levels in the plant. The AtD14 protein contains 3 amino acids which are referred to as the ‘catalytic triad’, namely S97 (Serine 97), D218 (Asparagine 218) and 11247 (Histidine 247). These are indicated in FIG. 2 for the Arabidopsis D14 protein and the corresponding amino acids in the modified watermelon ClD14 protein.

The watermelon protein of SEQ ID NO: 1 (ClD14ins), identified as underlying the multibranching phenotype (caused by the HMBN allele) was found to comprises a duplication of 8 amino acids. The duplication comprises one of the amino acids of the catalytic triad, as seen in FIG. 2, namely S97 is duplicated. S97 in the AtD14 protein appears to be located on the surface of the protein and seems to be involved in ligand binding.

Initially it was speculated by the applicants that, without being bound by any theory, it could be that the duplication of 8 amino acids in the ClD14 protein changes the ClD14 conformation to reduce or prevent the interaction with other proteins/ligands or changes the ClD14 conformation in such a way that the binding pocket for the strigolactone molecule is affected, thereby reducing or preventing signal transduction.

However, further analysis surprisingly found that the effect of the duplication of the 8 amino acids is that the ClD14ins protein is non-functional in vivo and cannot fulfill its signal transduction role in watermelon. The phenotype seen when the mutant allele is in homozygous form is, therefore, the most extreme secondary branch formation, referred herein as ‘full multibranching’ or ‘strong multibranching’. This was concluded from a TILLING mutant in which the codon for amino acid W155 was mutated into a STOP codon (W155STOP, or W155*), leading to a truncated protein comprising only amino acids 1 to 154 of SEQ ID NO: 2. The W155* protein must be non-functional, as 113 amino acids of the wild type protein are missing. The effect on (average) secondary branch formation in plants homozygous for the mutant allele W155* was the same as seen in the plants comprising the mutant allele encoding the ClD14ins protein. See Examples.

It was, therefore, surprisingly, found that the ClD4ins protein (comprising the 8 amino acid duplication) is non-functional in vivo, i.e. it lost its function in the strigolactone signalling pathway and does not transmit any signal anymore, whereby the inhibition of secondary branch formation is not induced and the multibranching phenotype is expressed to its fullest extent.

Watermelon plants are grown for fruit production are either diploid (2n), producing seeded fruits after pollination of the female flowers with pollen of the male flowers, or triploid (3n), producing seedless fruit after pollination of the female flowers with pollen from another watermelon plant (called a pollenizer plant), as the flowers of the triploid plants do not produce fertile pollen.

The HMBN allele has so far been used to develop pollenizer plants which contain the HMBN allele in homozygous form and have a multibranching phenotype. One of these pollenizers is the variety Sidekick (Harris Moran, see World wide web at hmclause.com/wp-content/uploads/2014/11/USACANADA_Watennelon_Sidekick_Techsheet_2014_ENG.pdf). Sidekick is a non-harvestable pollenizer, as the seeded fruits have pink fruit flesh and are discarded.

Commercial pollenizers can be distinguished as being harvestable or non-harvestable pollenizers (see also McGregor and Waters 2014, supra). Harvestable pollenizers are diploid pollenizers, which produce marketable, seeded fruits upon pollination of the female flowers. Non-harvestable pollenizers are diploid pollenizers which produce agronomically undesirable fruits upon pollination of the female flowers, such as white fleshed fruits, fruits having a brittle rind, etc. A grower can thus choose to produce triploid, seedless fruits and diploid, seeded fruits in one field, or to only produce triploid, seedless fruits and discarding the diploid, seeded fruits of the pollenizer. Obviously pollenizers take up a lot of space in the field, which can otherwise be occupied by triploid plants, and for this reason several pollenizers have been developed which produce compact plants.

The present inventors have found that the single recessive gene present in Sidekick and underlying the multibranching phenotype of Sidekick encodes a protein which comprises a duplication of 8 amino acids in comparison to the wild type protein of SEQ ID NO: 2. The modified (or mutant) protein is included herein as SEQ ID NO: 1. An alignment of the wild type protein and mutant protein are shown in FIG. 1 (‘D14/ns’ is the mutant protein of SEQ ID NO: 1 and ‘WT’ is the wild type protein of SEQ ID NO: 2). The genomic DNA and the cDNA/mRNA of the mutant ClD14 gene (shown in SEQ ID NO: 5 and SEQ ID NO: 3) therefore comprise a duplication of 24 nucleotides with respect to the wild type genomic DNA and the cDNA/mRNA (shown in SEQ ID NO: 6 and SEQ ID NO: 4).

The present inventors further found that the multibranching phenotype of Sidekick is due to the protein, which comprises a duplication of 8 amino acids in comparison to the wild type protein of SEQ ID NO: 2, being non-functional in vivo and that the phenotype is ‘full multibranching’, i.e. no signal transduction to inhibit secondary branch formation takes place. Thereby, the inventors for the first time are able to make different mutant alleles (different from the mutant allele present in Sidekick), which result in full multibranching when the mutant allele is in homozygous form, but also to make mutant alleles which retain function in vivo, but have reduced function compared to the wild type protein, and result in more moderate or intermediate multibranching when the mutant allele is in homozygous from.

By BLAST analysis the corresponding proteins of cucumber (CsD14) and melon (CmD14) were identified. These have a very high sequence identity to each other (using Emboss Needle pairwise alignments, default parameters), as shown in Table 1 below.

TABLE 1

| | ClD14 | CmD14 | CsD14 |
|---|---|---|---|
| | (SEQ ID | (SEQ ID | (SEQ ID |
| | NO: 2) | NO: 9) | NO: 8) |
| ClD14 (SEQ ID NO: 2) | 100% | | |
| CmD14 (SEQ ID NO: 9) | 98.5% | 100% | |
| CsD14 (SEQ ID NO: 8) | 98.1% | 99.6% | 100% |

Given the high protein sequence identity, it is expected that the in vivo function of the watermelon, cucumber and melon D14 proteins is the same.

Therefore, accordingly, the duplication of amino acids 94 to 101 in ClD14 (SEQ ID NO: 2), CsD14 (SEQ ID NO: 8) and CmD14 (SEQ ID NO: 9) should, in homozygous form, result in significantly more secondary branches (‘full multibranching’) being formed than in plants homozygous for the wild type allele, encoding the wild type protein. Likewise, other mutant alleles which result in a loss-of-function of the D14 protein, should result in ‘full multibranching’ and mutant alleles which result in a reduced-function of the D14 protein should result in ‘intermediate multibranching’. FIG. 3 shows a multiple sequence alignment of the mutant watermelon ClD14 protein (SEQ ID NO: 1, ClD14ins in FIG. 3) and the wild type cucumber and melon proteins.

In one aspect these mutant alleles and plants and plant parts (such as fruits) comprising these mutant alleles in homozygous or heterozygous form are encompassed herein.

Thus any mutant allele in the ClD14, CsD14 or CmD14 gene and a plant comprising such a mutant allele is encompassed herein, especially mutant alleles whereby one or more amino acids are inserted, deleted duplicated or replaced with respect to the wild type protein of SEQ ID NO: 2 (watermelon ClD14), SEQ ID NO: 8 (cucumber CsD14) or SEQ ID NO: 9 (melon CmD14). In one aspect the insertion, deletion, duplication or replacement of one or more amino acids results in the encoded protein being a reduced function D14 protein or loss-of-function D14 protein in vivo. In one aspect the mutant allele encodes a protein which comprises a duplication of at least 1, 2, 3, 4, 5, 6, 7 or of all 8 amino acids of amino acids 94 to 101 of SEQ ID NO: 2, SEQ ID NO: 8 or SEQ ID NO: 9. In one aspect at least the Ser (S) at position 97 of ClD14, CsD14 or CmD14 is duplicated.

Further methods for generating mutant alleles in the ClD14, CsD14 or CmD14 gene are encompassed herein. Especially a method for generating mutant alleles encoding a protein which has reduced function or loss of function in vivo and results in full multibranching (in case of loss of function in vivo) or intermediate multibranching (in case of reduced function in vivo), when the mutant allele is in homozygous form.

Also encompassed is, in one aspect, a method for generating mutant alleles encoding a protein which comprises a duplication of at least 1, 2, 3, 4, 5, 6, 7 or of all 8 amino acids of amino acids 94 to 101 of SEQ ID NO: 2, SEQ ID NO: 8 or SEQ ID NO: 9. In one aspect a method for generating a mutant allele whereby the allele encodes a protein wherein at least the Ser (S) at position 97 of the wild type ClD14, CsD14 or CmD14 protein is duplicated.

Also methods for screening (e.g. genotyping) and/or selecting plants or plant parts or seeds for the presence of mutant and/or wild type alleles of the ClD14, CsD14 or CmD14 gene are provided herein.

A mutant ClD14. CsD14 or CmD14 allele may comprise an allele encoding a protein wherein one or more amino acids are inserted, duplicated, deleted and/or replaced compared to the wild type ClD14, CsD14 or CmD14 protein, or a mutant ClD14, CsD14 or CmD14 allele may comprise one or more mutations (insertions, duplications, deletions and/or replacements of one or more nucleotides) in a regulatory region of the gene, such as a promoter or enhancer, resulting in reduced or no functional wild type protein being made.

In one aspect the mutant allele encodes a protein which comprises one or more amino acids replaced, inserted and/or deleted, whereby the protein is non-functional in vivo and a plant homozygous for the mutant allele shows full multibranching. Full multibranching is, thus, the complete lack of inhibition of secondary branch formation as no functional D14 is present in the plant Full multibranching in watermelon is, for example, seen as about 240% of the average number of secondary branches relative to the wild type plant (set to 100% secondary branching), see Examples. Preferably the phenotype of the plant comprising a mutant allele in homozygous form and a plant comprising a wild type allele in homozygous form is compared in the same genetic background, so that the background genome is highly similar and minimizes genotype differences.

In one aspect the mutant allele encodes a wild type D14 protein and the mutant allele is not expressed in vivo due to e.g. mutations in the regulatory region (such as the promoter) and a plant homozygous for the mutant allele shows full multibranching.

Knock-out alleles of D14 or mutant alleles of D14, whereby the mutation results in a loss-of-function of the D14 protein in vivo can be easily generated de novo, as will be explained elsewhere herein.

In one aspect the mutant allele encodes a protein which comprises one or more amino acids replaced, inserted and/or deleted, whereby the protein has a reduced function in vivo and a plant homozygous for the mutant allele shows intermediate multibranching. Intermediate multibranching is, thus, not the complete lack of inhibition of secondary branch formation in the plant, but the mutant D14 protein retains some functionality in vivo and partially inhibits secondary branch formation. Intermediate multibranching in watermelon is, for example, seen as the average number of secondary branches that develop being in between the average number of the wild type plant (homozygous for the functional D14 allele) and the average number of the plant homozygous for the non-functional D14 protein or for the knock-out allele. For example, if the plant homozygous for the functional D14 allele produces an average number of secondary branches that is set to 100%, and the plant homozygous for the allele encoding a non-functional D14 protein (or homozygous for a knock-out allele) produces 240% of secondary branches relative to the wild type, then the ‘intermediate multibranching’ produces an average number of secondary branches that is in-between 100% homozygous wild type) and 240% (homozygous non-functional), so about at least 110%, 120%, 130%, 140%, 150%, 160%, 170%, 180%, 190%, or 200% of the average number of secondary branches relative to the wild type plant (set to 100% secondary branching), but less than the full multibranching, see Examples. Mutant alleles in the D14 gene, whereby the mutation results in a reduced function in vivo of the D14 protein can be easily generated de novo, as will be explained elsewhere herein.

In one aspect the mutant allele encodes a protein which comprises one or more amino acids replaced, inserted and/or deleted in the IPR000073 domain, starting at amino acid 22 and ending at amino acid 259 of SEQ ID NO: 2 (watermelon), SEQ ID NO: 8 (cucumber) or SEQ ID NO: 9 (melon).

In one aspect the mutant allele encodes a mutant D14 protein as shown in Table 2 or in FIG. 6.

In one aspect the mutant allele encodes a protein which comprises one or more amino acids replaced, inserted and/or deleted in the region of amino acids 94 to 101 of SEQ ID NO: 2 (wild type watermelon protein), or of SEQ ID NO 8 (wild type cucumber protein) or of SEQ ID NO: 9 (wild type melon protein).

In one aspect the mutant allele encodes a protein which comprises one or more amino acids duplicated selected from amino acids 94 to 101 of SEQ ID NO: 2 (wild type watermelon protein), or of SEQ ID NO: 8 (wild type cucumber protein) or of SEQ ID NO: 9 (wild type melon protein).

In one aspect the mutant allele encodes a protein which comprises 1, 2, 3, 4, 5, 6, 7 or all 8 amino acids duplicated selected from amino acids 94 to 101 of SEQ ID NO: 2 (wild type watermelon protein ClD14), or of SEQ ID NO: 8 (wild type cucumber protein CsD14) or of SEQ ID NO: 9 (wild type melon protein CmD14). In one aspect the mutant allele encodes a protein which comprises at least a duplication of Serine 97 (S97) of SEQ ID NO: 2 (wild type watermelon protein), or of SEQ ID NO: 8 (wild type cucumber protein) or of SEQ ID NO: 9 (wild type melon protein). In one aspect the one or more duplicated amino acids are adjacent to the wild type amino acids.

In one aspect the mutant alleles described above result in increased secondary branching of the watermelon, cucumber or melon plant when the mutant allele is in homozygous form, compared to the plant which is homozygous for the wild type alleles (encoding the wild type proteins of ClD14, CsD14 and CmD14). In one aspect the mutant allele is a knock-out allele or encodes a non-functional D14 protein, resulting in full multibranching when the mutant allele is in homozygous form. In one aspect the mutant allele results in a mutant D14 protein having reduced function compared to the wild type D14 protein, but still retaining function in vivo, resulting in intermediate multibranching when the mutant allele is in homozygous form.

The above mutant alleles can easily be generated de novo by e.g. targeted gene editing techniques, such as CRISPR based techniques or by mutagenesis, such as radiation induced mutagenesis or chemically induced mutagenesis. A plant homozygous for the mutant allele can be generated by selfing the plant and then growing the homozygous plant in comparison to the wild type control (e.g. the non-mutated plant) to determine whether secondary branch numbers are higher in the homozygous mutant plant.

In another aspect the mutant ClD14, CmD14 or CsD14 allele encodes a truncated protein, whereby at least 10, 20, 30, 40, 50, 60, 70, 80, 90, 100, 110, 120, 130, 140, 150, 160, 170, 180, 190, 200 or more amino acids of the C-terminal end of the wild type ClD14, CsD14 or CmD14 protein are missing or are optionally replaced by different amino acids, rendering the protein to have a reduced in vivo function or no in vivo function.

In a different aspect the mutant ClD4, CmD14 or CsD14 allele encodes a protein, whereby at least 1, 2, 3, 4, 5, 6, 7, 8, 9, 10, 20, 30, 40, 50, 60, 70, 80, 90, 100, 110, 120, 130, 140, 150, 160, 170, 180, 190, 200 or more amino acids are inserted into, or are duplicated or are replaced or deleted from the wild type ClD14, CsD14 or CmD14 protein, rendering the protein to have a reduced in vivo function or no in vivo function.

As mentioned before, the extent of multibranching is determined by the functionality of the mutant protein, so a non-functional mutant protein will result in the maximum level of multibranching (herein referred to as full or strong multibranching), while a reduced function mutant protein will result in a lower degree of multibranching (herein referred to as intermediate multibranching). There is, thus, a direct relationship between D14 functionality and the degree of multibranching. The skilled person can easily generate different mutant alleles and homozygous plants comprising the mutant alleles and then grow the plants and select the mutant allele which results in the desired degree of multibranching.

In one aspect of the invention a plant or plant cell is provided, characterized in that the plant or plant cell has decreased activity of a ClD14 protein. CsD14 protein or CmD14 protein compared to a corresponding wild type plant cell, wherein the ClD14, CsD14 or CmD14 protein of the wild type plant cell is encoded by nucleic acid molecules selected from the group consisting of:
a) nucleic acid molecules, which encode a protein with the amino acid sequence given under SEQ ID NO: 2 (watermelon ClD14), SEQ ID NO: 8 (cucumber CsD14) or SEQ ID NO: 9 (melon CmD14);
b) nucleic acid molecules, which encode a protein, the sequence of which has an identity of at least 95%, 96%, 97%, 98% or 99% with the amino acid sequence given under SEQ ID NO: 2 (watermelon ClD14), SEQ ID NO: 8 (cucumber CsD14) or SEQ ID NO: 9 (melon CmD14).
c) a nucleic acid molecule of SEQ ID NO: 4 or SEQ ID NO: 6 or a sequence comprising at least 95%, 96%, 97%, 98% or 99% sequence identity to SEQ ID NO: 4 or to SEQ ID NO: 6 and encoding a ClD14 protein;
d) a nucleic acid molecule of SEQ ID NO: 17 or SEQ ID NO: 15 or a sequence comprising at least 95%, 96%, 97%, 98% or 99% sequence identity to SEQ ID NO: 17 or to SEQ ID NO: 15 and encoding a CsD14 protein;
e) a nucleic acid molecule of SEQ ID NO: 18 or SEQ ID NO: 16 or a sequence comprising at least 95%, 96%, 97%, 98% or 99% sequence identity to SEQ ID NO: 18 or to SEQ ID NO: 16 and encoding a CmD14 protein.

The decreased activity of the ClD14, CsD14 or CmD114 protein is caused by a mutant ClD14, CsD14 or CmD14 allele.

Decreased activity may be caused by a knock-down or knock-out of the expression of the mutant allele (e.g. through a mutation in the promoter or other regulatory sequence) or through the mutant allele encoding a loss-of-function or decreased-function ClD14, CsD14 or CmD14 protein. A mutant allele encoding a loss-of-function protein or a knock-out allele will, in homozygous form, result in the plant having a strong multibranching phenotype, while a mutant allele encoding a reduced-function protein or a knock-down allele will, in homozygous form, result in an intermediate multibranching phenotype, in-between the plant homozygous for the wild type allele and the plant homozygous for the loss-of-function (or knock-out) allele.

In one aspect the mutant ClD14, CsD14 or CmD14 allele encodes a mutant ClD14, CsD14 or CmD14 protein having decreased function or loss-of-function compared to the wild type protein, e.g. the mutant protein comprises one or more amino acids replaced, deleted and/or inserted, or duplicated, compared to the wild type protein. In one aspect the mutant allele encodes a protein which comprises one or more amino acids replaced, deleted or inserted with respect to the wild type protein of SEQ ID NO: 2, 8 or 9, whereby the mutant protein has either a loss of function and results in strong multibranching (when in homozygous form) or a reduced function and results in intermediate multibranching (when in homozygous form).

Mutant alleles which result in truncated D14 proteins will generally result in a loss of function, such as the W155* mutant generated herein in watermelon. Also, the Q255* may result in a loss of function or reduced function, as the last 13 amino acids of the protein are missing, including 5 amino acids of the highly conserved IPR000073 domain.

Alleles encoding truncated proteins, or proteins comprising one or more amino acids replaced by another amino acid or deleted or inserted, can easily be generated and tested in vivo, to see the effect on multibranching when the alleles are in homozygous form. The mutants described in Table 2 and in the Table A below can also easily be generated in watermelon, melon or cucumber, or other mutants can be generated using known methods, such as random mutagenesis followed by e.g. TILLING, targeted mutagenesis methods, etc.

Also software programs such as SIFT or PROVEAN analysis can be used to make a prediction of the effect of amino acid insertions, deletions, or replacements on protein function, although this is only a prediction and still needs to be confirmed in vivo. For example, P245L is predicted to be ‘not tolerated’ by SIFT analysis and ‘deleterious’ by Provean analysis, meaning that function of the protein is predicted to be lost or reduced. For changes that are predicted to be ‘tolerated’ using SIFT or ‘neutral’ using Provean analysis, function is predicted to not change. However, as mentioned, the prediction does not need to be true (it is based on statistical models) and in vivo analysis is needed. Still, the tools can be useful to focus further analysis on mutant alleles that are predicted to have an effect on protein function.

TABLE A

| Mutation in SEQ ID NO: 2 (watermelon | | |
|---|---|---|
| D14 protein), SEQ ID NO: 8 (cucumber | | |
| D14 protein) or SEQ ID NO: 9 (melon | SIFT | PROVEAN |
| D14 protein) | //sift.bii.a-star.edu.sg/ | //provean.jevi.org/ |
| V14I | tolerated | neutral |
| P44S | tolerated | neutral |
| L72F | Not tolerated | neutral |
| H89Y | tolerated | neutral |
| G121S | tolerated | neutral |
| S139N | tolerated | neutral |
| (in ID NO: 8 this is already an N) | | |
| W155Stop | Not tolerated | deleterious |
| G235V | tolerated | deleterious |
| P254L | Not tolerated | deleterious |
| Q255Stop | Not tolerated | deleterious |

One aspect herein is, therefore, a watermelon plant comprising a mutant allele of a gene named ClD14 (Citrullus lanatus Dwarf14), wherein the mutant allele comprises a mutation in one or more regulatory sequences resulting in decreased gene expression or no gene expression compared to a corresponding wild type allele, or wherein the mutant allele encodes a protein comprising a deletion, truncation, insertion or replacement of one or more amino acids, compared to the protein encoded by the wild type allele, resulting in a reduced function or loss of function of the ClD14 protein, wherein the mutant allele results in said plant developing an increased average number of secondary branches when the mutant allele is in homozygous form, and wherein the mutant allele is not the mutant allele which encodes the protein of SEQ ID NO: 1,
wherein the ClD14 protein of the wild type allele is encoded by nucleic acid molecules selected from the group consisting of:
a) nucleic acid molecules, which encode a protein with the amino acid sequence given under SEQ ID NO: 2;
b) nucleic acid molecules, which comprise the nucleotide sequence shown under SEQ ID NO: 6 or a complimentary sequence thereof.

Another aspect herein is a cucumber plant comprising a mutant allele of a gene named CsD14 (Cucumis sativus Dwarf14), wherein the mutant allele comprises a mutation in one or more regulatory sequences resulting in decreased gene expression or no gene expression compared to a corresponding wild type allele, or wherein the mutant allele encodes a protein comprising a deletion, truncation, insertion or replacement of one or more amino acids, compared to the protein encoded by the wild type allele, resulting in a reduced function or loss of function of the CsD14 protein, wherein the mutant allele results in said plant developing an increased average number of secondary branches when the mutant allele is in homozygous form, wherein the CsD14 protein of the wild type allele is encoded by nucleic acid molecules selected from the group consisting of:
a) nucleic acid molecules, which encode a protein with the amino acid sequence given under SEQ ID NO: 8;
b) nucleic acid molecules, which comprise the nucleotide sequence shown under SEQ ID NO: 15 or a complimentary sequence thereof.

Yet another aspect herein is a melon plant comprising a mutant allele of a gene named CmD14 (Cucumis melo Dwarf14), wherein the mutant allele comprises a mutation in one or more regulatory sequences resulting in decreased gene expression or no gene expression compared to a corresponding wild type allele, or wherein the mutant allele encodes a protein comprising a deletion, truncation, insertion or replacement of one or more amino acids, compared to the protein encoded by the wild type allele, resulting in a reduced function or loss of function of the CmD14 protein, wherein the mutant allele results in said plant developing an increased average number of secondary branches when the mutant allele is in homozygous form,
wherein the CmD14 protein of the wild type allele is encoded by nucleic acid molecules selected from the group consisting of:
a) nucleic acid molecules, which encode a protein with the amino acid sequence given under SEQ ID NO: 9;
b) nucleic acid molecules, which comprise the nucleotide sequence shown under SEQ ID NO: 16 or a complimentary sequence thereof.

Especially, the watermelon plant, cucumber plant or melon plant, comprises a mutant allele which encodes a protein in which one or more amino acids are inserted, replaced or deleted, wherein said mutant protein comprises a reduced function, but not a loss-of-function of the protein, whereby the average number of secondary branches is higher than in a plant which is homozygous for the wild type D14 allele, but not as high as in a plant homozygous for a mutant D14 allele encoding a non-functional protein.

Also in one aspect, the watermelon plant, cucumber plant or melon plant, comprises a mutant allele which encodes a protein in which one or more amino acids are inserted, replaced or deleted, wherein said mutant protein comprises a loss-of-function of the protein.

In one aspect the mutant allele encodes a protein which comprises V14 of SEQ ID NO: 2, 8 or 9 is replaced by a different amino acid, especially by I, or by a stop codon.

In one aspect the mutant allele encodes a protein which comprises P44 of SEQ ID NO: 2, 8 or 9 is replaced by a different amino acid, especially by S, or by a stop codon.

In one aspect the mutant allele encodes a protein which comprises L72 of SEQ ID NO: 2, 8 or 9 is replaced by a different amino acid, especially by F, or by a stop codon.

In one aspect the mutant allele encodes a protein which comprises H89 of SEQ ID NO: 2, 8 or 9 is replaced by a different amino acid, especially by Y, or by a stop codon.

In one aspect the mutant allele encodes a protein which comprises G121 of SEQ ID NO: 2, 8 or 9 is replaced by a different amino acid, especially by S, or by a stop codon.

In one aspect the mutant allele encodes a protein which comprises S139 of SEQ ID NO: 2 or 9 is replaced by a different amino acid, especially by N, or by a stop codon.

In one aspect the mutant allele encodes a protein which comprises W155 of SEQ ID NO: 2, 8 or 9 is replaced by a different amino acid or by a stop codon.

In one aspect the mutant allele encodes a protein which comprises G235 of SEQ ID NO: 2, 8 or 9 is replaced by a different amino acid, especially by V, or by a stop codon.

In one aspect the mutant allele encodes a protein which comprises P254 of SEQ ID NO: 2, 8 or 9 is replaced by a different amino acid, especially by L. or by a stop codon.

In one aspect the mutant allele encodes a protein which comprises Q255 of SEQ ID NO: 2, 8 or 9 is replaced by a different amino acid or by a stop codon.

In one aspect the mutant allele encodes a protein which comprises a duplication of at least 1, 2, 3, 4, 5, 6, 7, or all 8 amino acids selected from amino acid 94 to amino acid 101 of SEQ ID NO: 2, 8 or 9. In one aspect at least S97 is duplicated. In one aspect amino acids 94 to 101 are duplicated.

In another aspect the mutant allele encodes a protein which comprises a deletion or replacement of at least 1, 2, 3, 4, 5, 6, 7, or all 8 amino acids selected from amino acid 94 to amino acid 101 of SEQ ID NO: 2, 8 or 9. In one aspect at least S97 is deleted or replaced by another amino acid. In one aspect amino acid 94 to 101 are deleted or replaced by other amino acids.

In one aspect the mutant allele, thus, encodes a ClD14, CsD14, CmD14 protein of SEQ ID NO: 2, 8 or 9, respectively, in which at least S97 is duplicated, or in which at least 1, 2, 3, 4, 5, 6, 7, or all 8 consecutive amino acids of the following amino acids are duplicated V94 (Valine 94), G95 (Glycine 95), 196 (Histidine 96), S97 (Serine 97), V98 (Valine 98), S99 (Serine 99), A100 (Alanine 100), M101 (Methionine 101). In one aspect the at least 1, 2, 3, 4 or more consecutive amino acids include S97.

In one aspect the duplication of at least 1, 2, 3, 4 or more amino acids is located adjacent to the original amino acids, i.e. without an interval of other amino acids in between the duplicated amino acids.

In another aspect, the mutant allele encodes a ClD14, CsD14, CmD14 protein of SEQ ID NO: 2, 8 or 9, respectively, in which at least one amino acid, e.g. at least one amino acid selected from amino acids 94 to 101 of SEQ ID NO: 2, 8 or 9, or at least one amino acid in the IPR000073 domain of SEQ ID NO: 2, 8 or 9, or at least one amino acid of the helical lid domain, is replaced by another amino acid or by a STOP codon, resulting in a loss of function or decreased function protein and the phenotypic change (increased secondary branching) when the allele is in homozygous form (when no wild type allele is present in the diploid plant or plant cell). The IPR000073 domain starts at amino acid 22 of SEQ ID NO: 2, 8 and 9 and ends at amino acid 259 of SEQ ID NO: 2, 8 and 9. The helical lid domain starts at amino acid 136 of SEQ ID NO: 2, 8 and 9 and ends at amino acid 193 of SEQ ID NO: 2, 8 and 9. When referring to a start or an end, the mentioned amino acid or nucleotide is included.

In yet another aspect the mutant allele encodes a ClD14, CsD14, CmD14 protein of SEQ ID NO: 2, 8 or 9, respectively, in which at least one amino acid of the catalytic triad or preceding or following an amino acid of the catalytic triad by 1, 2, 3, 4, 5, 6, 7 or 8 positions, is replaced by another amino acid or by a STOP codon, resulting in a loss of function or decreased function protein and the phenotypic change (increased secondary branching) when the allele is in homozygous form (when no wild type allele is present in the diploid plant or plant cell). The amino acids of the catalytic triad are S97, D218 and 11247 of SEQ ID NO: 2, 8 or 9.

In another aspect one or more amino acids are missing, e.g. through a mutation causing a premature STOP codon, resulting in a loss of function or decreased function protein and the phenotypic change (increased secondary branching) when the allele is in homozygous form (when no wild type allele is present in the diploid plant or plant cell). In particular, in one aspect one or more amino acids selected from amino acids 94 to 101 of SEQ ID NO: 2, 8 or 9, are missing, e.g. through a premature STOP codon mutation being present in the sequence prior to the codon(s) encoding said amino acid(s). Or one or more amino acids of the IPR000073 domain are missing, or one or more amino acids of the helical lid domain are missing, or one or more amino acids of the catalytic triad and/or preceding or following an amino acid of the catalytic triad by 1, 2, 3, 4, 5, 6, 7, or 8 positions, are missing, e.g. through a premature STOP codon mutation being present in the sequence prior to the codon(s) encoding said amino acid(s).

A reduced function or a loss-of function of the protein is present when the mutant allele changes the in vivo phenotype from the wild type phenotype, i.e. normal secondary branching when the wild type allele is present in homozygous form, into increased secondary branching when the mutant allele is in homozygous form in a diploid plant. The term ‘increased secondary branching’ or ‘increased average number of secondary branches’, therefore, encompasses both a ‘full multibranching’ phenotype, caused by a loss-of-function D14 protein or knock-out of expression of the D14 allele, and a ‘intermediate multibranching’ phenotype caused by a reduced-function D14 protein or reduced expression of the D14 allele, compared to the wild type, functional D14 allele.

The absolute average number of secondary branches may differ somewhat between genotypes, but the relative effect should be the same in different genotypes. Thus, in a specific genetic background or genotype, the wild type has a certain average number of secondary branches, the loss-of-function protein or knock-out allele has the maximum or ‘full’ average number of secondary branches, and the reduced-function or knock-down allele is in-between these two extremes. E.g. if the average number of secondary branches in the wild type are set to be 100% and the loss-of-function is 240% relative to the wild type, then an average secondary branching above 100% and below 240% are ‘intermediate multibranching’ phenotypes. In one aspect, the ‘increased average secondary branching’ is at least 110%, 120%, 130%, 140%, 150%, 160%, 170%, 180%, 190%, 200%, 210% relative to the wild type (being 100%). In one aspect the ‘increased average secondary branching’ is lower than the ‘full multibranching’, it is e.g. 95% or less, 90% or less, 85% or less, 80% or less, 70% or less, 60% or less, 50% or less of the ‘full multibranching’ (being 100%).

In one aspect increased secondary branching is an average number of secondary branches of equal to or above 45, as seen in the watermelon plant homozygous for the allele encoding the protein of SEQ ID NO: 1 (comprising a duplication of amino acids 94 to 101 of SEQ ID NO: 2), compared to the watermelon plant homozygous for the wild type allele encoding the protein of SEQ ID NO: 2, which produces on average around 20 secondary branches. See also the Examples.

In watermelons and other cucurbits the main stem grows and forms primary lateral branches. On the primary lateral branches the plant makes secondary lateral branches. These secondary branches are counted starting at e.g. 90 cm from the end/crown on the main stem to the end/crown. Secondary branching is, thus, in one aspect measured by counting the number of secondary branches starting at a distance of 90 cm from the crown to the end/crown of the plant. This is done for several (at least 4, 5, 6, 7, 8, 9, 10) plants of a line and the average number of secondary branches is then calculated for each line. However, secondary branching can also be measured by counting the number of secondary branches starting at a shorter distance from the crown. e.g. 40 cm.

### SUMMARY

A cultivated watermelon, cucumber or melon plant or plant part is provided comprising at least one copy of a mutant allele of a gene named ClD14 in watermelon, CsD14 in cucumber or CmD14 in melon, said mutant allele conferring an average increased number of secondary branches when the mutant allele is in homozygous form in a diploid plant.

In one aspect the watermelon ClD14 gene is located on chromosome 8 of the watermelon genome, especially the gene is located in a region starting at base 28794281 and ending at base 28795173 of chromosome 8 of the Charleston Grey chromosome (cucurbitgenomics.org). The promoter sequence is located upstream of the genomic coding sequence, e.g. within the 1000 or 2000 bases upstream from base 28794281.

In one aspect the mutant ClD14, CsD14 or CmD14 allele confers, when in homozygous form, full multibranching which is the highest average number of secondary branches being formed, due to the encoded mutant protein being non-functional or due to the mutant allele not being expressed, i.e. being a knock-out allele.

In another aspect the mutant ClD14. CsD14 or CmD14 allele confers, when in homozygous form, intermediate multibranching which is an increased average number of secondary branches being formed compared to the wild type plant, but not the highest average number of branches capable of being formed in the full multibranching plant. The intermediate multibranching is due to the encoded mutant protein having a reduced function compared to the wild type protein or due to the mutant allele being expressed at a lower level than the wild type allele, i.e. being a knock-down allele.

In one embodiment the plant or plant part or seed comprising the mutant allele of the ClD14 gene is a watermelon plant or plant part or seed and is diploid, tetraploid, triploid or polyploid Preferably the mutant allele is present in one or two copies in a diploid plant or plant part or seed. Optionally it may be present in two or four copies in a tetraploid plant or plant part or seed, or in one, two or three copies in a triploid plant or plant part or seed.

The plant, plant part or seed may be watermelon comprising at least one copy of a mutant allele of a gene named ClD14, whereby the wild type gene encodes the wild type protein of SEQ ID NO: 2 (or a wild type protein comprising at least 95%, 96%, 97% or 98% sequence identity to SEQ ID NO: 2), or may be cucumber comprising at least one copy of a mutant allele of a gene named CsD14 whereby the wild type gene encodes the wild type protein of SEQ ID NO: 8 (or a wild type protein comprising at least 95%, 96%, 97% or 98% sequence identity to SEQ ID NO: 8), or may be melon comprising at least one copy of a mutant allele of a gene named CmD14, whereby the wild type gene encodes the wild type protein of SEQ ID NO: 9 (or a wild type protein comprising at least 95%, 96%, 97% or 98% sequence identity to SEQ ID NO: 9).

The plant part comprising the mutant allele of the ClD14, CsD14 or CmD14 gene may be a cell, a flower, a leaf, a stem, a cutting, pollen, a root, a rootstock, a scion, a fruit, a protoplast, an embryo, an anther.

Also encompassed is a vegetatively propagated watermelon, cucumber or melon plant propagated from such a plant part comprising at least one mutant allele of the ClD14, CsD14 or CmD14 gene.

Likewise a seed from which a plant of the invention can be grown is provided.

Further, male or female flowers, ovaries, anthers and pollen or microspores produced by a plant according to the invention is provided.

A method of producing watermelon, cucumber or melon fruits is provided, said method comprising growing a diploid plant comprising one or two copies of a mutant allele of a ClD14, CsD14 or CmD14 gene. Mutant alleles are described elsewhere herein, and are D14 alleles which in homozygous form confer increased secondary branching (homozygous for the mutant allele) compared to normal secondary branching (homozygous for the wild type D14 allele).

A method of producing seedless watermelon fruits is provided, said method comprising growing a triploid plant and a diploid pollenizer plant, whereby the pollenizer plant comprising two copies of a mutant allele of a ClD14 gene, allowing pollination of the flowers of the triploid plant and optionally harvesting the seedless triploid fruits.

A method of producing seedless watermelon fruits is provided, said method comprising growing a triploid plant and a diploid pollenizer plant, whereby the triploid plant comprising one, two or three copies of a mutant allele of a ClD14 gene, allowing pollination of the flowers of the triploid plant and optionally harvesting the seedless triploid fruits.

Further method of producing seeded watermelon fruits is provided, said method comprising growing a diploid plant, whereby the diploid plant comprising one or two copies of a mutant allele of a ClD14 gene, allowing pollination of the flowers and optionally harvesting the seeded diploid fruits.

A method for growing watermelon, cucumber or melon plants is provided, comprising growing a diploid watermelon, cucumber or melon plant comprising one or two copies of a mutant allele of a ClD14, CsD14 or CmD14 gene, especially in a field or in a greenhouse or tunnel.

A method for production of a cultivated watermelon, cucumber or melon plant producing an (average) increased number of secondary branches (compared to a plant homozygous for the wild type D14 gene) is provided comprising the steps of:
a) introducing random or targeted mutations into one or more watermelon, cucumber or melon plants, plant parts or seeds; or providing a population of mutant plants or seeds (e.g. a TILLING population),
b) selecting a plant comprises a mutant allele of a (ClD14, CsD14 or CmD14 gene, e.g. a mutant allele which produces significantly reduced or no wild type ClD14, CsD14 or CmD14 protein (e.g. a knock-down or knock-out allele) or which encodes a protein which comprises one or more amino acids deleted, replaced, inserted or duplicated compared to the wild type protein,
c) optionally removing any transgenic construct (e g. CRISPR construct) from the plant, and/or
d) optionally generating a plant homozygous for the mutant allele and analyzing the average number of secondary branches produced by the plant in comparison to the plant homozygous for the wild type allele.

A method for selecting or identifying watermelon, cucumber or melon plants, seeds or plant parts is provided comprising the steps of:
a) analyzing whether the genomic DNA of the plant or plant part comprises a mutant allele and/or comprises a wild type allele of the ClD14, CsD14 or CmD14 gene in their genome and optionally
b) selecting a plant or plant part comprising one or two copies of a mutant allele of the ClD14, CsD14 or CmD14 gene in the genome,

wherein the wild type allele of the ClD14 gene encodes the protein of SEQ ID NO: 2, the wild type allele of the Cs14 gene encodes the protein of SEQ ID NO: 8 and the wild type allele of the CmD14 gene encodes the protein of SEQ ID NO: 9.

Step a) can be carried out in various ways, using e.g. PCR based methods, sequencing based methods, nucleic acid hybridization based methods, gene expression levels, etc. In one aspect for example a KASP assay may be used, see e.g. Examples

A method for screening (e.g. genotyping) genomic DNA of watermelon, cucumber or melon plants, seeds or plant parts is provided comprising the steps of:
a) providing a sample (or a plurality of samples) of genomic DNA of a watermelon, melon or cucumber plant or of a plurality of plants (e.g. a F2 population, inbred lines, a backcross population, a breeding population, hybrid plants, etc.),
b) providing a pair of PCR primers or an oligonucleotide probe, which primers or (oligonucleotide) probe comprise at least 10, 11, 12, 13, 14, 15, 16, 17, 18, 19, 20, 21, 22 or more consecutive nucleotides of the genomic D14 allele of the ClD14, CsD14 or CmD14 gene and can hybridize to the genomic allele and/or amplify part of the genomic allele in a PCR assay, and
c) carrying out a PCR assay using the primer pair or a hybridization assay using the probe of step b) on the sample(s) of step a), and optionally
d) selecting a plant or plant part or seed comprising one or two copies of an allele (e.g. a wild type allele and/or a mutant allele) of the ClD14, CsD14 or CmD14 gene in the genome,

wherein the wild type allele of the ClD14 gene encodes the protein of SEQ ID NO: 2, the wild type allele of the Cs14 gene encodes the protein of SEQ ID NO: 8 and the wild type allele of the CmD14 gene encodes the protein of SEQ ID NO: 9.

In step b) a PCR primer pair is at least one forward primer, complementary to one of the DNA strands of the D14 allele and one reverse primer complementary to the other DNA strand of the D14 allele, which primer pair hybridizes to the denatured genomic DNA and amplifies part of the D14 allele in a PCR reaction. Primers can be designed to amplify the wild type or any mutant D14 allele using primer design tools. In one aspect two forward primers are used, one designed to amplify the wild type allele and one designed to amplify a mutant allele of the D14 gene, and one common reverse primer. These three primers can be used in a KASP-assay to genotype the samples of step a). Thus, in one aspect the assay in step c) is a KASP-assay, but also other genotyping assays can be used, such as those described in world wide web at biosearchtech.com/sectors/agrigenomics/agrigenomics-pcr-qpcr-technologies.

In one aspect the assay discriminates between a wild type and a mutant allele of the D14 gene. e.g. between the wild type ClD14 allele of SEQ ID NO: 6 and the mutant ClD14ins allele of SEQ ID NO: 5, or another mutant allele. Examples of other mutant alleles are given in Table A and Table 2, but also any other mutant allele is encompassed. e.g. any mutant allele that, in homozygous form, significantly increases the average number of secondary branches developing compared to the control plant, e.g. the plant comprising the wild type allele in homozygous form. In one aspect the mutant allele is a knock-out allele or a mutant allele encoding a loss-of-function protein leading to strong multibranching and in another aspect the mutant allele is a knock-down allele or a mutant allele encoding a reduced-function protein, leading to intermediate multibranching. Thus any wild type and/or mutant allele of the D14 gene can be detected in the assay.

For analyzing the genomic DNA at least crude genomic DNA extraction may be necessary. The presence of a mutant allele or a wild type allele in the genomic DNA can be detected directly or indirectly. Directly may for example be by nucleic acid hybridization of e.g. oligonucleotide probes. Indirectly may for example be by nucleic acid amplification using e g. PCR primers which comprise e.g. a tail sequence attached to the primer and during PCR the allele-specific primer binds to the template DNA and elongates, thereby attaching the tail sequence to the newly synthesized strand and in subsequent PCR rounds a FRET cassette (fluorescent resonant energy transfer cassette) binds to the tail and emits fluorescence. The fluorescent signal can then be detected. This is used e.g. in the KASP-assay.

The mutant allele may differ from the wild type allele in various aspects. e.g. in the promoter sequence or in the protein coding sequence or in the intron/exon splice sites. The mutant allele may have a reduced gene expression or no gene expression or it may result in the production of a protein comprising one or more amino acids deleted, replaced, or inserted or duplicated compared to the wild type protein.

In one aspect the mutant allele is an allele encoding a protein comprising one or more amino acids replaced, inserted or deleted with respect to the functional proteins of SEQ ID NO: 2, 8 or 9, whereby the mutant protein has reduced function or no function in vivo.

In one aspect the mutant allele is an allele encoding a protein comprising one or more amino acids replaced, inserted or deleted selected from: any one or more of the amino acids of the conserved IPR00073 domain and/or any one or more amino acids of the helical lid domain and/or any one or more of the catalytic triad amino acids and/or any one or more of the 8 amino acids preceding or following a catalytic triad amino acid, with respect to the functional proteins of SEQ ID NO: 2, 8 or 9, whereby the mutant protein has reduced function or no function in vivo. Thus, not only the plants and plant parts comprising one or more of the mutant alleles described herein are encompassed herein, but also the assays which enable the detection of a plant or plant part comprising at least one of the mutant alleles described herein are encompassed. Therefore, any watermelon, cucumber or melon plant, seed or plant part or DNA therefrom can be analyzed for the presence of the wild type D14 allele or for the presence of at least one of any of the mutant D14 alleles described herein. For any mutant allele an assay can easily be developed, as it is well known how to make primers or probes for a particular mutant allele E.g. for the W155* allele, an assay can easily be designed to detect the presence of the allele in the genomic DNA derived from a watermelon plant.

In one aspect the mutant allele is an allele encoding a protein comprising a duplication of 1, 2, 3, 4, 5, 6, 7 or 8 amino acids of amino acids 94 to 101 of SEQ ID NO: 2, 8 or 9. In one aspect the mutant allele comprises a duplication of at least Ser 97 of SEQ ID NO: 2, 8 or 9. In one aspect the mutant allele comprises a duplication of all amino acids of amino acids 94 to 101 of SEQ ID NO: 2, 8 or 9.

In one aspect the plant or plant part is watermelon and the mutant allele encodes the protein of SEQ ID NO: 1 (D14ins). This mutant allele, which encodes the 8 amino acid duplication as described herein, can be detected as described herein.

In another aspect the plant or plant part is watermelon and the mutant allele encodes a mutant protein comprising one or more amino acids inserted, replaced or deleted resulting in a reduced function or loss-of function, but which is not the protein of SEQ ID NO: 1 (D14 ins). i.e. it is not the allele present in variety Sidekick. The plant does, therefore, not comprise the sequence of SEQ ID NO: 5 in its genome. In one aspect the plant only comprises one copy of the sequence of SEQ ID NO: 5 in its genome.

Also, methods of generating and/or selecting plants or plant parts comprising at least one mutant allele of the watermelon ClD14 gene, or of the cucumber CsD14 gene or of the melon CmD14 gene in their genome is provided.

In one aspect also a method for detecting the presence of a wild type allele and/or of a mutant allele of the watermelon ClD14 gene, or of the cucumber CsD14 gene or of the melon CmD14 gene in the genome is provided.

In one aspect a method for detecting whether a watermelon plant or plant part or seed comprises at least one copy of the wild type allele, encoding the protein of SEQ ID NO: 2 and/or comprises at least one copy of a mutant allele, encoding e.g. the protein of SEQ ID NO: 1, or any protein comprising one or more amino acids replaced, inserted or deleted with respect to the protein of SEQ ID NO: 2 (as described elsewhere), is provided and optionally selecting a plant, plant part or seed comprising at least one copy of a mutant allele, said mutant allele encoding e.g. the protein of SEQ ID NO: 1, or any protein comprising one or more amino acids replaced, inserted or deleted with respect to the protein of SEQ ID NO: 2 (as described elsewhere).

In another aspect a method for detecting whether a watermelon plant or plant part or seed comprises at least one copy of the wild type allele comprising SEQ ID NO: 6 and/or comprises at least one copy of a mutant allele comprising one or more nucleotides inserted, replaced or deleted with respect to SEQ ID NO: 6, whereby the encoded protein has a reduced function or loss of function in vivo.

Provided is, in one aspect, a method for detecting, and optionally selecting, a watermelon plant, seed or plant part comprising at least one copy of a mutant allele of a gene named ClD14 (Citrullus lanatus Dwarf14), comprising the steps of:
a) providing one or more genomic DNA samples of one or more watermelon plants, seeds or plant parts.
b) carrying out a genotyping assay, using the DNA samples of a) as template, that discriminates between the wild type ClD14 allele and the mutant ClD14 allele, wherein said genotyping assay is based on nucleic acid amplification making use of ClD14 allele-specific oligonucleotide primers, and/or wherein said genotyping assay is based on nucleic acid hybridization making use of ClD14 allele-specific oligonucleotide probes, and optionally
c) selecting a plant, seed or plant part comprising one or two copies of the mutant allele,

wherein the mutant ClD14 allele comprises one or more nucleotides inserted, duplicated, deleted or replaced with respect to the sequence of SEQ ID NO: 6, resulting in a mutant ClD14 protein which comprises one or more amino acids inserted, duplicated, deleted or replaced with respect to the sequence of SEQ ID NO: 2.

In the method in one aspect said ClD14 allele-specific oligonucleotide primers or said ClD14 allele-specific oligonucleotide probes is a primer or probe which comprise at least 10 nucleotides of SEQ ID NO: 6 or of the complement strand of SEQ ID NO: 6.

In this method in one aspect the mutant allele comprises at least one codon inserted or duplicated in the coding region of the allele, or at least one codon changed into another codon, or at least one codon deleted or changed into a STOP codon. For example, the mutant allele is a mutant allele as described in Table A or Table 2 herein. The mutant allele may be an allele which encodes a mutant D14 protein having a loss-of-function or a reduced-function, resulting in strong multibranching or intermediate multibranching, respectively, when the mutant allele is in homozygous form.

Also a KASP-assay (Kbioscience Kompetitive Allele specific PCR-genotyping Assay) is provided comprising two allele specific forward primers, e.g. the FAM primer of SEQ ID NO: 10, and the VIC primer of SEQ ID NO: 11, and a Common reverse primer, e.g. of SEQ ID NO: 12. See also Examples. Obviously, other allele specific primers can be developed to detect and/or discriminate between the wild type allele (encoding the protein of SEQ ID NO: 2) and the mutant allele comprising a duplication of 24 nucleotides (encoding 8 amino acids) and encoding the protein of SEQ ID NO: 1, or any other mutant allele comprising e.g. one or more amino acids replaced, duplicated, deleted or inserted with respect to the wild type protein. For example, a KASP assay is provided to detect the mutant allele in which the codon for W155 is changed into a stop codon, or a KASP assay for any of the mutant alleles of Table 2, as well as any other mutant allele which results in a loss-of-function or reduced-function D14 protein in vivo.

Likewise, isolated sequences or molecules of the (wild type or mutant) genomic sequence, the cDNA or mRNA sequence, protein sequences, as well as oligonucleotide primers or probes for detecting a wild type or mutant allele of the watermelon ClD14 gene, or of the cucumber CsD14 gene or of the melon CmD14 gene are encompassed herein.

Also a method for generating a PCR amplification product and/or a oligonucleotide hybridization product of (a part of the) genomic DNA of watermelon, cucumber or melon plants, seeds or plant parts is provided comprising the steps of:
a) providing a sample (or a plurality of samples) of genomic DNA of a watermelon, melon or cucumber plant or of a plurality of plants (e.g. a F2 population, inbred lines, a backcross population, a breeding population, hybrid plants, etc.),
b) providing at least a pair of PCR primers or at least one oligonucleotide probe, which primers or (oligonucleotide) probe comprise at least 10, 11, 12, 13, 14, 15, 16, 17, 18, 19, 20, 21, 22 or more consecutive nucleotides of the genomic D14 allele of the ClD14, CsD14 or CmD14 gene and can hybridize to the genomic allele and/or amplify part of the genomic allele in a PCR assay, and
c) carrying out a PCR assay using the primer pair or a hybridization assay using the probe of step b) on the sample(s) of step a) to generate a PCR amplification product and/or an oligonucleotide hybridization product, and optionally
d) selecting a plant or plant part or seed comprising one or two copies of an allele (e.g. a wild type allele and/or a mutant allele) of the ClD14, CsD14 or CmD14 gene in the genome,

wherein the wild type allele of the ClD14 gene encodes the protein of SEQ ID NO: 2 (or comprises the genomic DNA of SEQ ID NO: 6), the wild type allele of the Cs14 gene encodes the protein of SEQ ID NO: 8 (or comprises the genomic DNA of SEQ ID NO: 15) and the wild type allele of the CmD14 gene encodes the protein of SEQ ID NO: 9 (or comprises the genomic DNA of SEQ ID NO: 16).

Further a method for amplifying and/or hybridizing (a part of the) genomic DNA of watermelon, cucumber or melon plants, seeds or plant parts is provided comprising the steps of:
a) providing a sample (or a plurality of samples) of genomic DNA of a watermelon, melon or cucumber plant or of a plurality of plants (e.g. a F2 population, inbred lines, a backcross population, a breeding population, hybrid plants, etc.),
b) providing at least a pair of PCR primers or at least one oligonucleotide probe, which primers or (oligonucleotide) probe comprise at least 10, 11, 12, 13, 14, 15, 16, 17, 18, 19, 20, 21, 22 or more consecutive nucleotides of the genomic D14 allele of the ClD14, CsD14 or CmD14 gene and can hybridize to the genomic allele and/or amplify part of the genomic allele in a PCR assay, and
c) carrying out a PCR assay using the primer pair or a hybridization assay using the probe of step b) on the sample(s) of step a) to generate a PCR amplification product and/or a oligonucleotide hybridization product, and optionally
d) selecting a plant or plant part or seed comprising one or two copies of an allele (e.g. a wild type allele and/or a mutant allele) of the ClD14, CsD14 or CmD14 gene in the genome, wherein the wild type allele of the ClD14 gene encodes the protein of SEQ ID NO: 2 (or comprises the genomic DNA of SEQ ID NO: 6), the wild type allele of the Cs14 gene encodes the protein of SEQ ID NO: 8 (or comprises the genomic DNA of SEQ ID NO: 15) and the wild type allele of the CmD14 gene encodes the protein of SEQ ID NO: 9 (or comprises the genomic DNA of SEQ ID NO: 16).

Also a genotyping kit comprising primers and/or probes and reaction components to amplify and/or hybridize part of the genomic DNA of the D14 gene is provided.

Primers and probes are preferably labelled or modified by e.g. a tail sequence or label, to be able to detect the amplification or hybridization reaction products.

### General Definitions

The verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”, e.g. “a plant” refers also to several cells plants, etc. Similarly, “a fruit” or “a plant” also refers to a plurality of fruits and plants.

As used herein, the term “plant” includes the whole plant or any parts or derivatives thereof, preferably having the same genetic makeup as the plant from which it is obtained, such as plant organs (e.g. harvested or non-harvested frits, leaves, flowers, anthers, etc.), plant cells, plant protoplasts, plant cell tissue cultures from which whole plants can be regenerated, plant calli, plant cell clumps, plant transplants, seedlings, plant cells that are intact in plants, plant clones or micropropagations, or parts of plants, such as plant cuttings, embryos, pollen, anthers, ovules, fruits (e.g. harvested tissues or organs), flowers, leaves, seeds, clonally propagated plants, roots, stems, root tips, grafts (scions and/or root stocks) and the like. Also any developmental stage is included, such as seedlings, cuttings prior or after rooting, etc. When “seeds of a plant” are referred to, these either refer to seeds from which the plant can be grown or to seeds produced on the plant, after self-fertilization or cross-fertilization.

As used herein, the term “variety” or “cultivar” means a plant grouping within a single botanical taxon of the lowest known rank, which can be defined by the expression of the characteristics resulting from a given genotype or combination of genotypes.

The term “allele(s)” means any of one or more alternative forms of a gene at a particular locus, e.g. the D14 locus (where the D14 gene is located; the alleles of the gene may be wild type alleles designated ClD14 (in watermelon) or CsD14 (in cucumber) or CmD14 (in melon), or mutant alleles, which alleles relate to one trait or characteristic at a specific locus (e.g. secondary branching). In a diploid cell of an organism, alleles of a given gene are located at a specific location, or locus (loci plural) on a chromosome. One allele is present on each chromosome of the pair of homologous chromosomes. A diploid plant species may comprise a large number of different alleles at a particular locus. These may be identical alleles of the gene (homozygous) or two different alleles (heterozygous), e.g. two identical copies of the mutant or one copy of the mutant allele and one copy of the wild type allele. Likewise a triploid plant is referred to as homozygous for the gene if it has three identical alleles of a gene (e.g. three copies of the mutant allele) and a tetraploid plant is referred to as homozygous for the gene if it has four identical alleles of the gene, e.g. four copies of the mutant allele.

“ClD14 gene” is a single, recessive gene identified in cultivated watermelon on chromosome 8, which when mutated results in a phenotypic change of an increased (average) number of secondary branches developing when the mutant allele is in homozygous form, compared to the plant homozygous for the wild type, non-mutated ClD14 gene. The CsD14 gene and CmD14 gene are the orthologs of the ClD114 gene, but then in cucumber and melon.

“F1, F2, F3, etc.” refers to the consecutive related generations following a cross between two parent plants or parent lines. The plants grown from the seeds produced by crossing two plants or lines is called the F1 generation. Selfing the F1 plants results in the F2 generation, etc.

“F1 hybrid” plant (or F1 hybrid seed) is the generation obtained from crossing two inbred parent lines. Thus, F1 hybrid seeds are seeds from which F1 hybrid plants grow. F1 hybrids are more vigorous and higher yielding, due to heterosis. Inbred lines are essentially homozygous at most loci in the genome.

A “plant line” or “breeding line” refers to a plant and its progeny. As used herein, the term “inbred line” refers to a plant line which has been repeatedly selfed and is nearly homozygous. Thus, an “inbred line” or “parent line” refers to a plant which has undergone several generations (e.g. at least 4, 5, 6, 7 or more) of inbreeding, resulting in a plant line with a high uniformity.

The term “gene” means a (genomic) DNA sequence comprising a region (transcribed region), which is transcribed into a messenger RNA molecule (mRNA) in a cell, and an operably linked regulatory region (e.g. a promoter). An example is the D14 gene of the invention. Different alleles of a gene are thus different alternatives form of the gene, which may be in the form of e.g. differences in one or more nucleotides of the genomic DNA sequence (e.g. in the promoter sequence, the exon sequences, intron sequences, etc.), mRNA and/or amino acid sequence of the encoded protein.

“Mutant ClD14 allele” refers herein to a mutant allele of the gene in watermelon, which causes the watermelon plant to develop an increased (average) number of secondary branches, e.g. equal to or more than 45 secondary branches, when the mutant allele is in homozygous form (also referred to as ‘multibranching’). Similarly, “mutant CsD14 allele or mutant CmD14 allele” refer to mutant alleles of the orthologous genes in cucumber and melon which cause in increase in secondary branching in these crops. The mutation in the mutant allele can be any mutation or combination of mutations, including deletions, truncations, insertions, duplications, point mutations, non-sense mutations, mis-sense mutations or non-synonymous mutations, splice-site mutations, frame shift mutations and/or mutations in one or more regulatory sequences such as promoter sequence, or enhancer or silencer sequences. The mutant ClD14 allele may result in ‘full multibranching’ or ‘strong multibranching’, which refers to the mutant allele not transmitting a signal in the plant to suppress secondary branch formation, due to the mutant allele encoding a non-functional protein or the mutant allele being a knock-out allele. The mutant ClD14 allele may result in ‘intermediate multibranching’, which refers to the mutant allele transmitting some signal in the plant to suppress secondary branch formation to some extent, but to a significantly lesser extent than in the wild type plant, due to the mutant allele encoding a reduced-functional protein or the mutant allele being a knock-down allele. The ‘intermediate multibranching’ phenotype is, therefore, in-between the average number of secondary branches of a plant homozygous for the wild-type, non-mutant allele and the average number of secondary branches of a plant having the ‘full multibranching’ phenotype.

“Wild type ClD14, or CsD14 or CmD14 allele” refers herein to the functional allele of the gene, which causes the plant to develop a normal number of secondary branches. The wild type ClD14 allele is found in any commercial variety of watermelon (e.g. Nunhems variety Premium F1, Montreal F1, and others). In one aspect the wild type ClD14 allele is a wild type allele of the ClD14 gene, whereby the ClD14 gene is the gene encoding a protein of SEQ ID NO: 2 or encoding a protein comprising at least 95%, 96%, 97%, 98% or 99% sequence identity to SEQ ID NO: 2 (when aligned pairwise, e.g. using Needle). In one aspect the wild type CsD14 allele is a wild type allele of the CsD14 gene, whereby the CsD14 gene is the gene encoding a protein of SEQ ID NO 8 or encoding a protein comprising at least 95%, 96%, 97%, 98% or 99% sequence identity to SEQ ID NO: 8 (when aligned pairwise, e.g. using Needle). In one aspect the wild type CmD14 allele is a wild type allele of the CmD14 gene, whereby the CmD14 gene is the gene encoding a protein of SEQ ID NO: 9 or encoding a protein comprising at least 95%, 96%, 97%, 98% or 99% sequence identity to SEQ ID NO: 9 (when aligned pairwise, e.g. using Needle).

The term “locus” (loci plural) means a specific place or places or a site on a chromosome where for example a gene or genetic marker is found. The ClD14 locus is, thus, the location in the genome of watermelon, where the mutant allele and/or the wild type allele of the ClD14 gene is found. The ClD14 locus is a locus on cultivated watermelon chromosome 8 (using the chromosome assignment of the published watermelon genome found at world wide web at cucurbitgenomics.org under “Watermelon: Genome”, “Charleston Grey” or “97103 V1 or V2).

“Induced mutant alleles” are mutant alleles in which the mutation(s) is/are/have been induced by human intervention. e.g. by mutagenesis via physical or chemical mutagenesis methods or via e.g. tissue culture (as described in e.g. Zhang et al, Plos 9(5) e96879), including also targeted gene editing techniques (such as Crispr based techniques, TALENS, etc.).

“Diploid plant” refers to a plant, vegetative plant part(s), or seed from which a diploid plant can be grown, having two sets of chromosome, designated herein as 2n.

A “DH plant” or “doubled-haploid plant” is a diploid plant produced by doubling the haploid genome of the diploid plant using e.g. in vitro techniques. A DH plant is, therefore, homozygous at all loci.

“Triploid plant” refers to a plant, vegetative plant part(s), or seed from which a triploid plant can be grown, having three sets of chromosomes, designated herein as 3n.

“Tetraploid plant” refers to a plant, vegetative plant part(s), or seed from which a tetraploid plant can be grown, having four sets of chromosomes, designated herein as 4n.

“Polyploid plant” refers to plants having a higher ploidy than diploid, i.e. triploid (3n), tetraploid (4n), hexaploid (6n), octaploid (8n), etc.

“Pollenizer plant” or “pollenizer” refers to the (inbred or hybrid) diploid plant, or parts thereof (e.g. its pollen or scion), suitable as pollenizer for inducing fruit set on triploid plants. A pollenizer plant is, thus, able to lead to good fruit set (and good triploid fruit yield) of normal triploid plants, by producing an appropriate amount of pollen at the appropriate day-time and for an appropriate period of time.

“Hybrid triploid plant” or “F1 triploid” or “triploid hybrid” is a triploid plant grown from hybrid, triploid seed obtained from cross fertilizing a male diploid parent with a female tetraploid parent. The male parent is used for inducing fruit set and seed production on a tetraploid female parent, resulting in fruits containing F1 hybrid triploid seeds. Both the male parent and the female parent used to produce F1 triploid seeds are inbred so that each parent line is nearly homozygous and stable.

“Seedless fruit” are fruits which contain no viable mature seeds. The fruit may contain one or more small, edible, white ovules. Optionally the fruit may contain a few brown or black seeds, but these are not viable. Viable mature seeds are seeds which can be germinated in soil under appropriate conditions and grow into plants.

“Interplanting” refers to the combination of two or more types of seeds and/or transplants sown or transplanted on the same field, especially the sowing and/or transplanting of pollenizers in the same field as triploid hybrid plants (for seedless fruit production on the triploid plants and diploid fruit production on the pollenizer plants). For example, the pollenizer may either be planted in separate rows or interplanted with the triploid plants in the same row (e.g. in hills within each row). Pollenizers may also be planted in between rows of triploids. Also seeds of pollenizers and triploid hybrids may be mixed prior to seeding, resulting in random seeding. The transplants of the triploid hybrid plants and/or pollenizer plants may also comprise a rootstock of a different plant. Suitable rootstocks are known in the art. Watermelon plants with a different rootstock are referred to as “grafted”.

“Planting” or “planted” refers to seeding (direct sowing) or transplanting seedlings (plantlets) into a field by machine or hand.

“Vegetative propagation” or “clonal propagation” refers to propagation of plants from vegetative tissue, e.g. by in vitro propagation or grafting methods (using scions and rootstocks). In vitro propagation involves in vitro cell or tissue culture and regeneration of a whole plant from the in vitro culture. Grafting involves propagation of an original plant by grafting onto a rootstock. Clones (i.e. genetically identical vegetative propagations) of the original plant can thus be generated by either in vitro culture or grafting. “Cell culture” or “tissue culture” refers to the in vitro culture of cells or tissues of a plant. “Regeneration” refers to the development of a plant from cell culture or tissue culture or vegetative propagation. “Non-propagating cell” refers to a cell which cannot be regenerated into a whole plant.

“Recessive” refers to an allele which expresses its phenotype (e.g. multibranching) when no dominant allele is present in the diploid genome, i.e. when it is homozygous in a diploid. The mutant ClD14 allele results in a plant having a phenotypic change (described elsewhere) when present in two copies in a diploid plant, optionally in four copies in a tetraploid plant or in two or three copies in a triploid plant or in the respective number of copies in another polyploidy. The dominant allele is herein also referred to as the wild type (WT) allele.

“Cultivated watermelon” or “Citrullus lanatus” refers herein to Citrullus lanatus ssp. vulgaris, or Citrullus lanatus (Thunb.) Matsum. & Nakai subsp. vulgaris (Schrad.), and having good agronomic characteristics, especially producing marketable fruits of good fruit quality and fruit uniformity. This excludes wild watermelon.

“Wild watermelon” refers herein to Citrullus lanatus ssp. lanatus and Citrullus lanatus ssp. mucosospermus, producing fruits of poor quality and poor uniformity.

“Cultivated cucumber” or “cultivated melon” refer to Cucumis sativus or Cucumis melo having good agronomic characteristics, especially producing marketable fruits of good fruit quality and fruit uniformity. This excludes wild cucumber or wild melons producing fruits of poor quality and poor uniformity.

“SNP marker” refers to a Single Nucleotide Polymorphism between e.g. a mutant ClD14, CsD14 or CmD14 allele and a wild type allele. Using a SNP marker assay which can distinguish between the mutant and wild type allele of the gene (i.e. an allele specific assay) one can screen pants, plant parts or the DNA therefrom for the presence of the mutant allele and/or the wild type allele.

“INDEL marker” refers to an insertion/deletion polymorphism between e.g. a mutant ClD14, CsD14 or CmD14 allele and a wild type ClD14, CsD14 or CmD14 allele. For example, the marker mWM23349015_k2 is an INDEL marker, which distinguishes between the wild type ClD14 allele, encoding the protein of SEQ ID NO: 2, and the mutant ClD14 allele, encoding the protein of SEQ ID NO: 1 (comprising a duplication of 8 amino acids). Using an INDEL marker assay which can distinguish between the mutant and wild type allele of the gene (i.e. an allele specific assay) one can screen pants, plant parts or the DNA therefrom for the presence of the mutant allele.

“Genotyping” methods are methods whereby the genotype or allelic composition of a plant or plant part or seed can be determined. Bi-allelic genotyping assays, such as KASP-assays, can distinguish between two alleles at a locus.

“Cultivated watermelon genome” and “physical position on the cultivated watermelon genome” and “chromosome 8” refers to the physical genome of cultivated watermelon, the reference genome is found on the world wide web at cucurbitgenomics.org under “Watermelon: Genome”, e.g. “Watermelon (Charleston Grey)” and the physical chromosomes and the physical position on the chromosomes.

A “chromosome region comprising the mutant ClD14 allele” refers to the genomic region of e.g. chromosome 8 of cultivated watermelon which region carries the mutant ClD14 allele. The presence of the allele can be determined phenotypically and/or by the detection of markers distinguishing different ClD14 alleles or by the genomic sequence of the allele sequence itself (determined e.g. by sequencing the allele). An “allele specific marker” is a marker which is specific for a particular allele (e.g. a specific mutant allele) and is thus discriminating between e.g. the mutant allele and the wild type allele.

A genetic element, an introgression fragment, or a gene or allele conferring a trait (such as the phenotypic characteristics of the mutant D14 allele) is said to be “obtainable from” or can be “obtained from” or “derivable from” or can be “derived from” or “as present in” or “as found in” a plant or seed or tissue or cell if it can be transferred from the plant or seed in which it is present into another plant or seed in which it is not present (such as wild type line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Cultivated watermelons containing the genetic element, locus, introgression fragment, gene or allele (e.g., a mutant ClD14 allele) can be generated de novo, e.g. by mutagenesis (e.g. chemical mutagenesis, CRISPR-Cas induced, etc.) and then e.g. be crossed into other cultivated watermelons. Similarly, cultivated cucumbers or melons containing the genetic element, locus, introgression fragment, gene or allele (e.g., a mutant CsD14 or CmD14 allele) can be generated de novo.

“Average” or “mean” refers herein to the arithmetic mean and both terms are used interchangeably. The term “average” or “mean” thus refers to the arithmetic mean of several measurements. The skilled person understands that the phenotype of a plant line or variety depends to some extent on growing conditions and that, therefore, arithmetic means of at least 4, 5, 6, 7, 8, 9, 10, 15, 20, 30, 40, 50 or more plants (or plant parts) are measured, preferably in randomized experimental designs with several replicates and suitable control plants grown under the same conditions in the same experiment. “Statistically significant” or “statistically significantly” different or “significantly” different refers to a characteristic of a plant line or variety that, when compared to a suitable control show a statistically significant difference in that characteristic (e.g. the p-value is less than 0.05, p<0.05, using ANOVA) from the (mean of the) control. For example, when referring herein to differences in average numbers of secondary branches it is understood that the differences referred to are statistically significant differences, e.g. an ‘intermediate multibranching’ plant genotype has a statistically significantly higher average number of secondary branches than the control plant genotype, comprising the wild type D14 allele in homozygous form.

The term “traditional breeding techniques” encompasses herein crossing, backcrossing, selfing, selection, double haploid production, chromosome doubling, embryo rescue, protoplast fusion, marker assisted selection, mutation breeding etc., all as known to the breeder, by which, for example, a chromosome 8 comprising a mutant ClD14 allele can be obtained, identified and/or transferred.

“Backcrossing” refers to a breeding method by which a (single) trait, such as the phenotypic changes conferred by the mutant ClD14 allele, can be transferred from one (often an inferior) genetic background (also referred to as “donor”) into another (often a superior) genetic background (also referred to as “recurrent parent”. An offspring of a cross (e g an F1 plant obtained by crossing e.g. the donor with the recurrent parent watermelon, or an F2 plant or F3 plant, etc., obtained from selfing the F1), is “backcrossed” to the parent with e.g. the superior genetic background. After repeated backcrossing, the trait of the one (often inferior) genetic background will have been incorporated into the other (often superior) genetic background.

“Marker assisted selection” or “MAS” is a process of using the presence of molecular markers (such as SNP markers or INDEL markers), which are genetically and physically linked to a particular locus or to a particular chromosome region or allele specific markers, to select plants for the presence of the specific locus or region or allele. For example, a molecular marker genetically and physically linked to the mutant ClD14 allele or an allele specific marker, can be used to detect and/or select e.g. watermelon plants, or plant parts, comprising the mutant ClD14 allele. Allele specific markers are preferred markers, as they select for the allele directly.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule.

A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

“Sequence identity” and “sequence similarity” can be determined by alignment of two peptide or two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm to align two sequences over their entire length, maximizing the number of matches and minimising the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using computer programs, such as EMBOSS as available on the world wide web under ebi.ac.uk/Tools/psa/emboss_needle/). Alternatively sequence similarity or identity may be determined by searching against databases such as FASTA, BLAST, etc., but hits should be retrieved and aligned pairwise to compare sequence identity. Two proteins or two protein domains, or two nucleic acid sequences have “substantial sequence identity” if the percentage sequence identity is at least 95%, 96%, 97%, 98%, 99% or more (as determined by Emboss “needle” using default parameters, i.e. gap creation penalty=10, gap extension penalty=0.5, using scoring matrix DNAFULL for nucleic acids and Blosum62 for proteins).

When reference is made to a nucleic acid sequence (e.g. DNA or genomic DNA) having “*substantial sequence identity to” a reference sequence or having a sequence identity of at least 95%, e.g. at least 96%, 97%, 98% or 99% nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions.

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions.

“M1 generation” or “M1 plants” in context with the present invention shall refer to the first generation that is produced directly from the mutagenic treatment. A plant grown from seeds treated with a mutagen e.g. is a representative of an M1 generation.

“M2 generation” or “M2 plant” shall refer herein to the generation obtained from self-pollination of the M1 generation. A plant grown from seeds obtained from a self-pollinated M1 plant represents a M2 plant. M3, M4, etc. refers to further generations obtained after self-pollination.

An “mRNA coding sequence” shall have the common meaning herein. An mRNA coding sequence corresponds to the respective DNA coding (cDNA) sequence of a gene/allele apart from that thymine (T) is replaced by uracil (U).

A “mutation” in a nucleic acid molecule (DNA or RNA) is a change of one or more nucleotides compared to the corresponding wild type sequence, e.g. by replacement, deletion or insertion of one or more nucleotides. Examples of such a mutation are point mutation, nonsense mutation, missense mutation, splice-site mutation, frame shift mutation or a mutation in a regulatory sequence.

A “nucleic acid molecule” shall have the common understanding in the art. It is composed of nucleotides comprising either of the sugars deoxyribose (DNA) or ribose (RNA).

A “point mutation” is the replacement of a single nucleotide, or the insertion or deletion of a single nucleotide.

A “nonsense mutation” is a (point) mutation in a nucleic acid sequence encoding a protein, whereby a codon in a nucleic acid molecule is changed into a stop codon. This results in a pre-mature stop codon being present in the mRNA and results in translation of a truncated protein. A truncated protein may have decreased function or loss of function.

A “missense or non-synonymous mutation” is a (point) mutation in a nucleic acid sequence encoding a protein, whereby a codon is changed to code for a different amino acid. The resulting protein may have decreased function or loss of function.

A “splice-site mutation” is a mutation in a nucleic acid sequence encoding a protein, whereby RNA splicing of the pre-mRNA is changed, resulting in an mRNA having a different nucleotide sequence and a protein having a different amino acid sequence than the wild type. The resulting protein may have decreased function or loss of function.

A “frame shift mutation” is a mutation in a nucleic acid sequence encoding a protein by which the reading frame of the mRNA is changed, resulting in a different amino acid sequence. The resulting protein may have decreased function or loss of function.

A “deletion” in context of the invention shall mean that anywhere in a given nucleic acid sequence at least one nucleotide is missing compared to the nucleic sequence of the corresponding wild type sequence or anywhere in a given amino acid sequence at least one amino acid is missing compared to the amino acid sequence of the corresponding (wild type) sequence.

A “truncation” shall be understood to mean that at least one nucleotide at either the 3′-end or the 5′-end of the nucleotide sequence is missing compared to the nucleic sequence of the corresponding wild type sequence or that at least one amino acid, but preferably at least 10, 20, 30, 40, 50, 60, 70, 80, 90, 100 or more amino acids, at either the N-terminus or the C-terminus of the protein is missing compared to the amino acid sequence of the corresponding wild type protein. The 5′-end is determined by the ATG codon used as start codon in translation of a corresponding wild type nucleic acid sequence.

“Replacement” shall mean that at least one nucleotide in a nucleic acid sequence or at least one amino acid in a protein sequence is different compared to the corresponding wild type nucleic acid sequence or the corresponding wild type amino acid sequence, respectively, due to an exchange of a nucleotide in the coding sequence of the respective protein.

“Insertion” shall mean that the nucleic acid sequence or the amino acid sequence of a protein comprises at least one additional nucleotide or amino acid compared to the corresponding wild type nucleic acid sequence or the corresponding wild type amino acid sequence, respectively.

“Duplication” shall mean that one or more (consecutive) nucleotides or one or more (consecutive) amino acids are present at least twice in a nucleotide or amino acid sequence in stead of once in the wild type sequence. A duplication is, therefore, an insertion of one or more consecutive nucleotides or one or more consecutive amino acids which were already present once in a wild type sequence. The insertion may be adjacent to the original sequence, or it may be separated by one or more nucleotides or amino acids, i.e. it may be duplicated further away from the original sequence.

“Pre-mature stop codon” in context with the present invention means that a stop codon is present in a coding sequence (cds) which is closer to the start codon at the 5′-end compared to the stop codon of a corresponding wild type coding sequence.

A “mutation in a regulatory sequence”, e.g. in a promoter or enhancer of a gene, is a change of one or more nucleotides compared to the wild type sequence, e.g. by replacement, deletion or insertion of one or more nucleotides, leading for example to decreased or no mRNA transcript of the gene being made.

A “mutation in a protein” is a change of one or more amino acid residues compared to the wild type sequence, e.g. by replacement, deletion, truncation or insertion or duplication of one or more amino acid residues.

“Mutant protein” is herein a protein comprising one or more mutations in the nucleic acid sequence encoding the protein, whereby the mutation results in (the mutant nucleic acid molecule encoding) a “reduced-function” or “loss-of-function” protein, as e.g. measurable in vivo. e.g. by the phenotype conferred by the mutant allele.

“Wild type 3-dimensional structure” or “wild type protein folding” refers to the in vivo folding of the wild type protein to carry out its normal function in vivo. “Modified 3-dimensional structure or modified protein folding” refers to the mutant protein having a different folding than the wild type protein, which reduces or abolishes its normal function or activity in vivo, i.e. the protein has a reduced-function or loss-of-function. Protein truncations also lead to a modified 3-dimensional structure. The 3-D structure can be predicted to be modified using e.g. programs such as RaptorX and comparing the predicted wild type protein structure to the predicted modified protein structure.

In context of the present invention, “decreased activity” of a protein shall mean a decrease in activity of a D14 protein when compared to a corresponding wild type plant cell or a corresponding wild type plant. Decrease shall in one aspect comprise an entire knock-out or knock-down of gene expression, or the production of a loss-of-function or of a reduced-function D14 protein, e.g. a mutant D14 protein may have lost function or decreased function compared to the wild type, functional D14 protein. A decrease in activity can be a decrease in the expression of a gene encoding a D14 protein (also referred to as knock-down), or a knock-out of the expression of a gene encoding a D14 protein and/or a decrease in the quantity of a D14 protein in the cells, or a reduced-function or loss-of-function in the activity of a D14 protein in the cells. As it was found that the D14 protein function directly reflects (and causes) the degree of secondary branching, the loss-of-function protein (or knock-out allele) or decreased-function protein (or knock-down allele) can be determined phenotypically, in a plant homozygous for the mutant allele and will be seen either in a ‘full multibranching’ phenotype or a ‘intermediate multibranching’ phenotype.

In context with the present invention, the term “wild type plant cell” or “wild type plant” means that they comprise wild type D14 alleles and not mutant D14 alleles. Thus, the wild type plant or wild type plant cell is a plant or plant cell comprising fully functional D14 genes, encoding a fully functional ClD14, CsD14 or CmD14 proteins (also referred to as wild type D14 protein), e.g. regarding watermelon plants or plant cells a diploid watermelon plant comprising in its genome SEQ ID NO: 6 and/or producing the protein of SEQ ID NO: 2 (or a protein comprising at least 95% sequence identity to SEQ ID NO: 2) and having a normal branching phenotype.

“Knock-out” or “entire knock-out” shall be understood that expression of the respective gene is not detectable anymore.

“Loss-of-function” or “reduced-function” or “decreased function” shall mean in context of the present invention that the protein, although possibly present in amounts equal or similar to a corresponding wild type protein, does not evoke its normal effect anymore, i.e. for mutant alleles encoding such a protein when present in homozygous form in a diploid plant, the plant produces a phenotypic change described elsewhere herein. As mentioned, it was found that the D14 protein function directly reflects (and causes) the degree of secondary branching, the loss-of-function protein or decreased-function protein can be determined phenotypically, in a plant homozygous for the mutant allele and will be seen either in a ‘full multibranching’ phenotype or a ‘intermediate multibranching’ phenotype.

“Catalytic triad” refer to 3 conserved amino acids in the wild type ClD14, CsD14 and CmD14 proteins, S97, D218 and H247 of SEQ ID NO: 2 (ClD14), SEQ ID NO: 8 (CsD14) and SEQ ID NO: 9 (CmD14). “Targeted gene editing” is referred to techniques whereby endogenous target genes can be modified, e.g. one or more nucleotides can be inserted, replaced and/or deleted e.g. in the promoter or coding sequence. For example CRISPR based techniques, such as Crispr-Cas9 gene editing, Crispr-Cpf1 gene editing, or more recent techniques called ‘base editing’ or ‘primer editing’ can be used to modify endogenous target genes, such as the endogenous wild type ClD14 gene in watermelon (encoding the protein of SEQ ID NO: 2 or a wild type protein comprising at least 95% sequence identity to SEQ ID NO: 2), the endogenous wild type CsD14 gene in cucumber (encoding the protein of SEQ ID NO: 8 or a wild type protein comprising at least 95% sequence identity to SEQ ID NO: 8) and the endogenous wild type CmD14 gene in melon (encoding the protein of SEQ ID NO: 9 or a wild type protein comprising at least 95% sequence identity to SEQ ID NO: 9).

“Oligonucleotides” or “oligos” or “oligonucleotide primers or probes” are short, single-stranded polymers of nucleic acid, e.g. at least 10, 11, 12, 13, 14, 15, 16, 17, 18, 19, 20, 21, 22, 23, 24 or more nucleotides in length. Oligos may be unmodified or modified with a variety of chemistries depending on their intended use, for example, the addition of 5′ or 3′ phosphate groups to enable ligation or block extension, respectively, labeling with radionuclides or fluorophores and/or quenchers for use as probes, the incorporation of thiol, amino, or other reactive moieties to enable the covalent coupling of functional molecules such as enzymes, and extension with other linkers and spacers of diverse functionality. DNA oligos are the most commonly used, but RNA oligos are also available. The length of an oligo is usually designated by adding the suffix -mer. For example, an oligonucleotide with 19 nucleotides (bases) is called a 19-mer. For most uses, oligonucleotides are designed to base-pair with a strand of DNA or RNA. The most common use for oligonucleotides is as primers for PCR (polymerase chain reaction). Primers are designed with at least part of their sequence complementary to the sequence targeted for amplification. Optimal primer length for a complementary sequence is e.g. 18 to 22 nucleotides. Optimal primer sequences for PCR are usually determined by primer design software.

“DNA microarrays” are arrays which have many microscopic spots of DNA, usually oligonucleotides, bound on a solid support. Assay targets can be DNA, cDNA, or cRNA. Depending on the system, the hybridization of targets to specific spots is detected by fluorescence, chemiluminescence, or colloidal silver or gold. Microarrays are used for multiple applications such as simultaneous measurement of the expression of large numbers of genes, enabling genome-wide gene expression analysis, as well as genotyping studies using e.g. single-nucleotide polymorphism (SNP) or InDel analysis.

“Complementary strands” refer to two strands of complementary sequence, and may be referred to as sense (or plus) and anti-sense (or minus) strands for double stranded DNA. The sense/plus strand is, generally, the transcribed sequence of DNA (or the mRNA that was generated in transcription), while the anti-sense/minus strand is the strand that is complementary to the sense sequence. For any of the sequences provided herein only one strand of the sequence is given, but the complementary strand of the given strand is also encompassed herein. The complementary nucleotides of DNA are A complementary to T, and G complementary to C. The complementary nucleotides of RNA are A complementary to U, and G complementary to C.

### FIGURES

FIG. 1: A pairwise amino acid sequence alignment between the wild type (WT) ClD14 protein of SEQ ID NO: 2 and the mutant ClD14ins protein of SEQ ID No: 1. The 8 duplicated amino acids are highlighted in bold.

FIG. 2: A pairwise amino acid sequence alignment between the Arabidopsis AtD14 protein (SEQ ID NO: 7) with the ClD14ins protein of SEQ ID NO: 1. The amino acids of the catalytic triad are highlighted in bold.

FIG. 3: Multiple sequence alignment of the watermelon ClD14ins protein of SEQ ID NO: 1 and the wild type cucumber CsD14 protein (SEQ ID NO: 8) and the wild type melon CmD14 protein (SEQ ID NO: 9).

FIG. 4: Pairwise alignment of the wild type genomic sequence (SEQ ID NO: 6), encoding the wild type watermelon ClD14 protein of SEQ ID NO: 2, and the mutant genomic sequence (SEQ ID NO: 5), comprising 24 duplicated/inserted nucleotides and encoding the mutant protein of SEQ ID NO: 1 (comprising a duplication of 8 amino acids, including one of the amino acids of the catalytic triad, S97). The intron sequence is indicated in bold.

FIG. 5: Allelic discrimination plot for InDel marker mWM23349015_k2, with Fam allele (mutant insertion allele) on X axis and VIC allele (wild type/deletion allele) on Y axis.

FIG. 6: TILLING mutants identified in the wild type ClD14 protein are shown in bold and underlined, with the amino acid substitution indicated below. Boxed amino acids are the amino acids of the catalytic triad. The light grey bar shows the helical lid domain from amino acid 136 to 193 (described in Seto et al., 2019, Nature Communications 10: 191). The two black triangles (with arrows) show the start and end of the conserved domain IPR00073 (amino acid 22 to 259), which is an InterPro domain described as ‘alpha/beta hydrolase fold-1’ or ‘AB_hydrolase_1” domain. This domain is described as follows. The α/β hydrolase fold is common to a number of hydrolytic enzymes of widely differing phylogenetic origin and catalytic function. The core of each enzyme is an α/β-sheet (rather than a barrel), containing 8 strands connected by helices. The enzymes are believed to have diverged from a common ancestor, preserving the arrangement of the catalytic residues. All have a catalytic triad, the elements of which are borne on loops, which are the best conserved structural features of the fold. The catalytic triad residues are presented on loops. One of these is the nucleophile elbow and is the most conserved feature of the fold.

FIG. 7: Right side photo of the W155Stop TILLING mutant (homozygous for the W155Stop allele), showing a multibranching phenotype. Left photo azygous plant (homozygous for the wild type allele), wherein the functional ClD14 protein binds strigolactone and suppresses secondary branching.

### DETAILED DESCRIPTION

A first embodiment of the present invention concerns cultivated watermelon, cucumber or melon plants, comprising at least one copy of a mutant allele of a gene, named herein D14 gene (ClD14, CsD14 or CmD14), conferring (when in homozygous form) a change the average number of secondary branches developing compared to the plant homozygous for the functional, wild type allele of the gene.

The ClD14 gene is an endogenous gene of cultivated watermelon, which when mutated and in homozygous form results in a significant increase in secondary branches being produced by the plant.

In a multibranching watermelon it was found that both copies of the endogenous allele of the ClD14 gene contained a duplication of 24 nucleotides in the coding sequence, which in turn lead to a duplication of 8 amino acids. The protein is called ClD4ins herein and is shown in SEQ ID NO: 1 The duplication included one of the amino acids of the catalytic triad (S97). Originally it was speculated by the inventors that this duplication might reduce the function or abolish the correct functioning of the catalytic triad in vivo.

The D14 is a complex protein with several functions in the plant and several functional domains in the protein, including strigolactone binding, hydrolosis, interaction with various other proteins and ligands, conformational changes and signal transduction.

It was, therefore, very surprising that a TILLING mutant, which produced a truncated, non-functional D14 protein (referred to as W155* protein), had the same phenotype as the protein comprising the 8 amino acid duplication. This meant that the protein comprising the 8 amino acid duplication (including the catalytic triad amino acid S97) actually was a loss-of-function protein and that the phenotype seen was the strongest secondary branching (referred to herein as ‘full multibranching’ or ‘strong multibranching’). It also meant that mutant proteins which do not have a complete loss-of-function can be generated, which cause an ‘intermediate multibranching’ phenotype, i.e. the signaling pathway whereby secondary branch formation is suppressed, is still induced and transmitted by a reduced-function D14 protein, so that there is only partial suppression of secondary branching.

Thus, in one aspect a watermelon plant comprising a mutant allele of a gene named ClD14 (Citrullus lanatus Dwarf14) is provided, wherein the mutant allele comprises a mutation in one or more regulatory sequences resulting in decreased gene expression or no gene expression compared to a corresponding wild type allele, or wherein the mutant allele encodes a protein comprising a deletion, truncation, insertion or replacement of one or more amino acids, compared to the protein encoded by the wild type allele, resulting in a reduced function or loss-of-function of the ClD14 protein, wherein the mutant allele results in said plant developing an increased average number of secondary branches when the mutant allele is in homozygous form, and wherein the mutant allele is not the mutant allele which encodes the protein of SEQ ID NO: 1 (ClD4ins protein), wherein the ClD14 protein of the wild type allele is encoded by nucleic acid molecules selected from the group consisting of:
a) nucleic acid molecules, which encode a protein with the amino acid sequence given under SEQ ID NO: 2
b) nucleic acid molecules, which comprise the nucleotide sequence shown under SEQ ID NO: 6 or a complimentary sequence thereof.

In one aspect the mutant allele encodes a protein in which one or more amino acids are inserted, replaced or deleted, resulting in a loss-of-function of the protein, whereby the average number of secondary branches is at its highest level (full multibranching), e.g. at least 200%, 210%, 215%, 220% or more of the wild type control plant comprising the wild type allele in homozygous form. e.g. it is as high as in a plant homozygous for a mutant ClD14 allele encoding a non-functional protein ClD4ins protein or the W155* protein, but the mutant allele is not the allele encoding the ClD14ins protein of SEQ ID NO: 1. The genome of the plant does, therefore, not comprise SEQ ID NO: 5 on chromosome 8, which is the genomic sequence encoding the ClD14ins protein.

ClD4 alleles which encode loss-of-function D14 proteins can be easily generated de novo. For example by random or targeted mutagenesis. Two specific mutant alleles are the W155* mutant and the Q255* mutant generated in the Examples. However, any other mutant allele, which results in a loss of ClD14 protein function is encompassed and can easily be generated and its phenotype tested.

In another aspect the mutant allele encodes a protein in which one or more amino acids are inserted, replaced or deleted, resulting in a reduced function of the protein, but not a loss-of-function of the protein, whereby the average number of secondary branches is higher than in a plant which is homozygous for the wild type ClD14 allele, but not as high as in a plant homozygous for a mutant ClD14 allele encoding a non-functional protein, such as for example the ClD14 ins protein or the W155* protein.

The watermelon plant is in one aspect homozygous for the mutant allele and develops an increased average number of secondary branches (full multibranching or intermediate multibranching) compared to the plant which is homozygous for the wild type allele. Also encompassed is a seed from which a plant having increased average secondary branching (full multibranching or intermediate multibranching) can be grown.

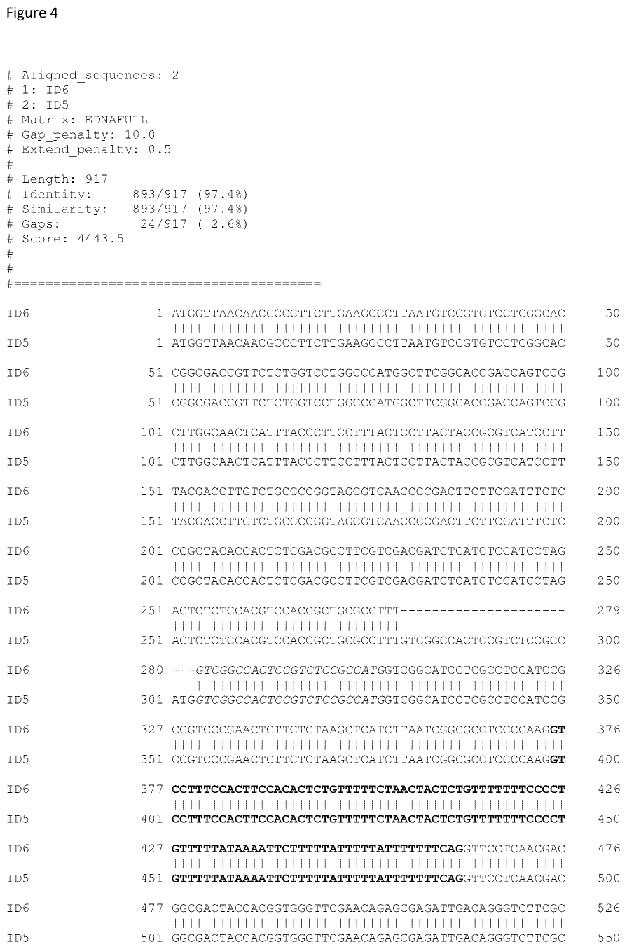

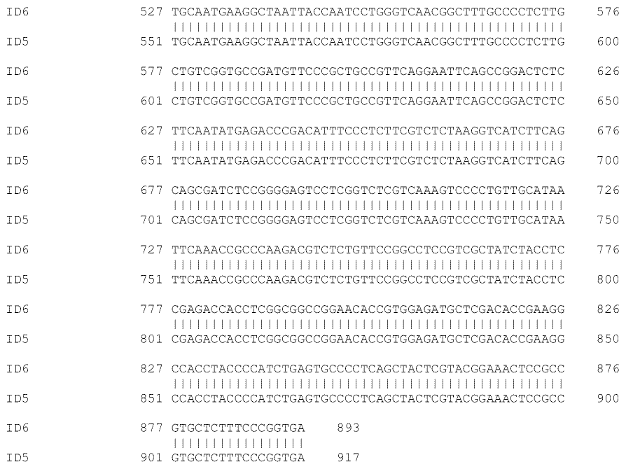

For the original multibranching mutant (comprising the 8 amino acid duplication, called herein the ClD14ins protein) a high throughput genotyping assay based on the INDEL marker (Insertion/Deletion) in the mutant allele, i.e. the insertion of 24 additional nucleotides in the mutant/modified allele and the “deletion” (absence) of these 24 nucleotides in the wild type allele, was developed to screen the genomic DNA of populations of plants, seeds or plant parts for the INDEL. FIG. 4 shows the genomic sequence of the ClD14 wild type allele (SEQ ID NO: 6; ‘Deletion of 24 nucleotides’) and mutant/modified allele (SEQ ID NO: 5, ‘Insertion of 24 nucleotides’).

The two sequences comprising the INDEL, and which were used to design the two forward and one reverse PCR primers, are shown in SEQ ID NO: 13 (‘deletion’ sequence, i.e. wild type allele) and SEQ ID NO: 14 (‘insert sequence’, i.e. mutant allele). These are sequences of the reverse strand (− strand) of the alleles. The forward strand (plus strand) is shown in SEQ ID NO: 6 (wild type genomic sequence) and SEQ ID NO: 5 (mutant genomic sequence with insert) and also in FIG. 4.

However, similar genotyping assays can be developed (and are encompassed herein) for any mutant allele of the D14 gene, e.g. any mutant shown in Table A or Table 2 or other mutant alleles of the ClD14 gene.

In one aspect, therefore, a genotyping assay is provided for genotyping watermelon plants, seeds, plant parts, cells or tissues, comprising the steps:
a) providing genomic DNA of one or more watermelon plants or a population of plants, and
b) carrying out a genotyping assay which detects the presence of the wild type allele of SEQ ID NO: 6 (or the complement strand thereof) and/or the presence of a mutant allele, wherein the mutant allele comprises one or more nucleotides inserted, deleted, replaced or duplicated with respect of SEQ ID NO: 6, and optionally
c) selecting a plant, seed, plant part, cell or tissue comprising either two copies of the wild type allele, or one copy of the wild type allele and one copy of a mutant allele, or two copies of a mutant allele.

In step b) the mutation in the mutant allele preferably causes one or more amino acids to be inserted, deleted or replaced with respect to the wild type protein.

In one aspect, a genotyping assay genotyping watermelon plants, plant parts, cells or tissues, comprising the steps is provided, comprising the steps:
a) providing genomic DNA of one or more watermelon plants or a population of plants (e.g. breeding population, F2 population, backcross population etc.), and
b) carrying out a genotyping assay which detects the presence of the wild type allele encoding the protein of SEQ ID NO: 2 and/or the presence of a mutant allele, wherein the mutant allele comprises one or more amino acids inserted, deleted, replaced or duplicated with respect of SEQ ID NO: 2, and optionally
c) selecting a plant, seed, plant part, cell or tissue comprising either two copies of the wild type allele, or one copy of the wild type allele and one copy of a mutant allele, or two copies of a mutant allele.

Step a) may comprise isolation of genomic DNA from the plant, seeds, plant part, cell or tissue to be analyzed in the genotyping assay. Often crude DNA extractions methods can be used, as known in the art.

Step b) preferably comprises a bi-allelic genotyping assay, which makes use of allele-specific primers and/or allele-specific probes.

The plants of step a) may be mutagenized using e.g. chemical or radiation mutagens or gene editing techniques. Thus prior to step a) there may be a step of treating the plants, seeds or plant parts with a mutagenic agent or induce targeted mutations in the ClD14 allele.

Various genotyping assays can be used, as long as they can detect INDELs and SNPs and can differentiate between the wild type allele of SEQ ID NO: 6 being present in the genomic DNA (at the ClD14 locus on chromosome 8) or a mutant allele of the ClD14 gene being present in the genomic DNA. Genotyping assays are generally based on allele-specific primers used in PCR or thermal cy cling reactions (polymerase chain reaction) to amplify either the wild type or mutant allele and detect the amplification product or on allele-specific oligonucleotide probes, which hybridize to either the wild type allele or the mutant allele, or both. For example genotyping with BHQplus probes uses two allele specific probes and two primers that flank the region of the polymorphism, and during thermal cycling the polymerase encounters the allele-specific probes bound to the DNA and releases a fluorescent signal. Allele discrimination involves competitive binding of the two allele-specific BHQPlus probes (see also biosearchtech com).

Examples of genotyping assays are the KASP-assay (by LGC, see www at LGCgenomics.com and also www at biosearchtech.com/products/pcr-kits-and-reagents/genotyping-assays/kasp-genotyping-chemistrv), based on competitive allele-specific PCR and end-point fluorescent detection, the TaqMan-assay (Applied Biosystems), which is also PCR based, HRM assays (High Resolution Melting Assay), wherein allele-specific probes are detected using real time PCR, or the rhAmp assay, based on Rnase H2-dependent PCR, BHQplus genotyping, BHQplex CoPrimer genotyping and many others.

The KASP-assay is also described in lie C. Holme J, Anthony J. ‘SNP genotyping: the KASP assay. Methods Mol Biol. 2014:1145:75-86’ and EP1726664B1 or U.S. Pat. No. 7,615,620 B2, incorporated by reference. The KASP genotyping assay utilizes a unique form of competitive allele-specific PCR combined with a novel, homogeneous, fluorescence-based reporting system for the identification and measurement of genetic variation occurring at the nucleotide level to detect single nucleotide polymorphisms (SNPs) or inserts and deletions (InDels). The KASP technology is suitable for use on a variety of equipment platforms and provides flexibility in terms of the number of SNPs and the number of samples able to be analyzed. The KASP chemistry functions equally well in 96-, 384-, and 1,536-well microtiter plate formats and has been utilized over many years in large and small laboratories by users across the fields of human, animal, and plant genetics.

The TaqMan genotyping assays is also described in Woodward J. ‘Bi-allelic SNP genotyping using the TaqMan® assay.’ Methods Mol Biol. 2014; 1145:67-74, U.S. Pat. Nos. 5,210,015 and 5,487,972, incorporated herein by reference. With TaqMan® technology allele-specific probes are utilized for quick and reliable genotyping of known polymorphic sites. TaqMan assays are robust in genotyping multiple variant types, including single nucleotide polymorphisms, insertions/deletions, and presence/absence variants. To query a single bi-allelic polymorphism, two TaqMan probes labeled with distinct fluorophores are designed such that they hybridize to different alleles during PCR-based amplification of a surrounding target region. During the primer extension phase of PCR, the 5′-3′ exonuclease activity of Taq polymerase cleaves and releases the fluorophores from bound probes. At the end of PCR, the emission intensity of each fluorophore is measured and allele determination at the queried site can be made.

Various genotyping assay s can, therefore, be used, which can differentiate between the presence of the wild type allele of the ClD14 gene, encoding the protein of SEQ ID NO: 2, or a mutant allele of the ClD14 gene. Various mutant alleles of the ClD4 gene can be detected. So, not only the mutant allele encoding the protein of SEQ ID NO: 1 (comprising 8 additional amino acids due to a duplication of 24 nucleotides), but the assay can be designed to detect any other mutant allele of the ClD14 gene, such as any mutant allele e.g. as described in Table A or Table 2 or others.

As mentioned preferably a bi-allelic genotyping assay is used, e.g. a KASP-assay, a TaqMan assay, a BHQplus assay, PACE genotyping (see world wide web at idtdna.com/pages/products/qpcr-and-pcr/genotyping/pace-snp-genotyping-assays) or any other bi-allelic genotyping assay.

In one aspect the genotyping assay in step b) of the methods above is a KASP-assay. Thus in step b) a competitive PCR is carried out using two forward primers and one common reverse primer. The two forward primers comprise at least 10, 11, 12, 13, 14, 15, 16, 17, 18, 19 or 20 nucleotides complementary to SEQ ID NO: 6 (or the complement strand thereof). In addition the two forward primers comprise 1, 2, 3 or more nucleotides (preferably at the 3′end of the primers) which provide specificity to the SNP or INDEL which differentiates the wild type sequence from the mutant sequence of the allele. The two forward primers thereby have different binding specificity (or preference) to either the wild type allele or to the mutant allele. For example the Fam-primer comprises 17 nucleotides of the wild type sequence and 1 nucleotide specific for the insertion allele, and the VIC-primer in the Examples comprises 18 nucleotides of the wild type allele and 1 nucleotide specific to the ‘deletion’ allele. A KASP-assay can easily be designed to differentiate between the wild type allele of SEQ ID NO: 6 and any mutant allele of the ClD14 gene which differs from the wild type allele in one or more nucleotides being inserted, deleted or replaced, so e.g. the assay can be designed for any SNP or INDEL that differentiates two alleles.

It is noted that genotyping assays, such as the KASP assay described e.g. in the Examples, can also be carried out to detect the mutant and/or wild type ClD14 allele in triploid or tetraploid watermelon plants and plant parts in the same way as described for diploid watermelon plants and plant parts.

In one aspect the mutant allele of the ClD14 gene encodes a protein comprising one or more amino acids inserted, duplicated, replaced or deleted with respect of the wild type protein of SEQ ID NO: 2.

In one aspect the mutant allele of the ClD14 gene encodes a protein which is truncated in comparison to the protein of SEQ ID NO: 2, e.g. at least 8, 10, 15, 20, 30, 40, 50, 60, 70, 80, 90, 100 or more amino acids are missing at the C-terminal end or optionally at the N-terminal end.

In one aspect the mutant allele of the ClD14 gene encodes a protein which comprises one or more amino acids deleted or replaced in comparison to the protein of SEQ ID NO: 2, e.g. at least 1, 2, 3, 4, 5.6, 7, 8, 9, 10 or more amino acids are deleted or replaced by one or more different amino acids.

In another aspect the mutant allele of the ClD14 gene encodes a protein which comprises one or more amino acids inserted or duplicated in comparison to the protein of SEQ ID NO: 2, e.g. at least 1, 2, 3, 4, 5, 6, 7, 8, 9, 10, 11, 12, 13, 14, 15 or more amino acids are inserted or duplicated. In one aspect at least one or more amino acids of amino acid 94 to amino acid 101 of SEQ ID NO: 2 are duplicated, preferably at least the S97 is duplicated. In one aspect at least 2, 3, 4, 5, 6, 7 or 8 consecutive amino acids of amino acids 94 to 101 of SEQ ID NO: 2 are duplicated, preferably wherein the consecutive amino acids include the S97.

Therefore, in one embodiment a method is provided for detecting, and optionally selecting, a watermelon plant, seed or plant part comprising at least one copy of a wild type allele and/or of a mutant allele of a gene name ClD14 (Citrullus lanatus Dwarf14), comprising:
a) providing genomic DNA of a watermelon plant or of a plurality of plants (e.g. a breeding population, F2, backcross, etc.),
b) carrying out an assay (e.g. a bi-allelic genotyping assay) that discriminates or can discriminate between the presence of alleles in the genomic DNA of a), based on nucleic acid amplification (e.g. comprising the use of allele specific oligonucleotide primers) and/or nucleic acid hybidization (e.g. comprising the use of allele-specific oligonucleotide probes), to detect the presence of a wild type allele of the gene and/or a mutant allele of the gene, wherein the wild type allele comprises the sequence of SEQ ID NO: 6 (or wherein the wild type allele encodes the protein of SEQ ID NO 2) and the mutant allele comprises one or more nucleotides inserted, duplicated, deleted or replaced with respect to the sequence of SEQ ID NO: 6 (or the mutant allele encodes a protein comprising one or more amino acids inserted, duplicated, deleted or replaced with respect to the wild type protein of SEQ ID NO: 2), and optionally
c) selecting a plant, seed or plant part comprising one or two copies of the mutant allele.

Under step b) the genotyping assay discriminates between the wild type and the mutant alleles based on nucleic acid (especially DNA) amplification reactions making use of e.g. oligonucleotide primers, such as PCR (Polymerase Chain Reaction) and PCR primers, preferably allele-specific primers, and/or nucleic acid hybridization making use of as oligonucleotide probes, preferably allele-specific probes.

The primers or probes are preferably modified to comprise a label, e.g. a fluorescent label, or to comprise a tail sequence or other modification.

In one aspect, in any of the above methods the assay uses one or more ClD14 allele specific primers or one or more ClD14 allele specific probes. As mentioned, based on the genomic sequence of SEQ ID NO: 6 or other (e.g. degenerate) genomic sequences which encode the protein of SEQ ID NO: 2 or the genomic sequence of a mutant allele which encodes e.g. a protein comprising one or more amino acids inserted, duplicated, deleted or replaced in comparison to SEQ ID NO: 2, PCR primers and nucleic acid probes can be designed using known methods or software programs for oligonucleotide design. Primers and probes may for example be at least 10, It, 12, 13, 14, 15, 16, 17, 18, 19, 20, 21, 22, 23, 24 or more nucleotides (bases) in length and anneal to (or hybridize to) the template DNA sequence, i.e. they preferably have at least 95%, 96%, 97%, 98%, 99% or 100% sequence identity to the target sequence. The primer or probe specificity to a wild type allele or a mutant allele is due to at least 1, 2, 3 or more nucleotides of the primer or probe being specific for either allele. The primers or probes are thus designed around the polymorphism (e.g. the SNP or InDel) between the two alleles of the target gene, so that they discriminate between these. In one aspect the assay is a bi-allelic genotyping assay selected from e.g. a KASP-assay, a TaqMan-assay, a BHQplus probe assay or any other bi-allelic genotyping assay.

In one aspect, the mutant allele comprises at least one codon inserted or duplicated in the coding region of the allele, or at least one codon changed into another codon (e.g. through a single nucleotide change), or at least one codon deleted or changed into a STOP codon.

In any of the methods above, in one aspect the mutant allele comprises the sequence of SEQ ID NO: 5. i.e. comprises an insertion/duplication of 24 nucleotides, leading to the duplication of 8 amino acids in the protein.

Thus, in one aspect the methods can be used to discriminate between plants, seeds or plant parts comprising two copies of the wild type ClD14 allele encoding the protein of SEQ ID NO: 2, two copies of the mutant ClD14 allele encoding the protein of SEQ ID NO: 1, or one copy of each allele (heterozygous). Optionally plants, plant parts or seeds comprising any of these genotypes may be selected for e.g. further breeding or for use in watermelon production.

In any of the methods above, in another aspect the mutant allele encodes a mutant protein described herein, e.g. in Table A or Table 2. Thus, in one aspect the methods can be used to discriminate between plants, seeds or plant parts comprising two copies of the wild type ClD14 allele encoding the protein of SEQ ID NO: 2, two copies of the mutant ClD14 allele encoding the mutant protein described herein, e.g. in Table A or Table 2, or one copy of each allele (heterozygous). Optionally plants, plant parts or seeds comprising any of these genotypes may be selected for e.g. further breeding or for use in watermelon production.

Thus, in one aspect, in any of the above methods, the mutant allele encodes a loss-of-function protein or a reduced-function protein, as described.

Although any DNA genotyping assay may be used in the above methods, be it PCR based (using PCR primers) and/or hybridization based (using probes), in one aspect a KASP-assay is used to discriminate between the wild type and the mutant allele. The assay can be used in a high throughput way, e.g. in 96 well plates or more well plates (e.g. 384 well plates).

Depending on the SNP or INDEL, between the wild type and mutant ClD14 allele, various allele-specific primers and probes can be designed for use in the assays.

In one aspect two forward primers (one for the wild type allele and one for the mutant allele) and one common reverse primer (for both the wild type and the mutant allele) are used in the KASP-assay. In one aspect the two forward primers and the reverse primer comprise at least 10, 11, 12, 13, 14, 15, 16, 17, 18, 19 or more nucleotides of SEQ ID NO: 6 or of the complement sequence of SEQ ID NO: 6. The forward primers further comprise at least 1, 2, or 3 nucleotides (preferably at the 3′end of the primer) which confer specificity (or preference) to either amplification of the wild type allele or amplification of the mutant allele. Each forward primer forms a primer pair with the common reverse primer to amplify the DNA sequence of the target allele in between the primer pair, during thermal cycling. Standard components for thermal cy cling are used and standard components for KASP-assays.

In one aspect the KASP-assay discriminates between the InDel found in the ClD14 allele, i.e. the KASP-assay can discriminate between the presence in the genomic DNA of SEQ ID NO: 6 in homozygous form (ClD14 wild type, normal branching allele), the presence of SEQ ID NO: 5 in homozygous form (ClD14 allele with insertion, multibranching allele) and the presence of both SEQ ID NO: 6 and SEQ ID NO: 5 in the watermelon genome. Different forward and reverse primers can be designed to achieve allele discrimination in the assay.

In one aspect the forward primers comprise the sequence of SEQ ID NO: 10 and/or SEQ ID NO: 11, or the complement sequence of either of these. In one aspect the common primer optionally comprises the sequence of SEQ ID NO: 12 or the complement sequence thereof.

In one aspect the primers comprise one or more of SEQ ID NO: 10 (forward primer), SEQ ID NO: 11 (forward primer), and SEQ ID NO: 12 (common primer), or a sequence comprising at least 95%, 96%, 97%, 98%, 99% sequence identity to SEQ ID NO: 10, SEQ ID NO: 11 or SEQ ID NO: 12, or a complementary sequence of any one of these sequences.

In another embodiment a method is provided for producing a hybridization product or an amplification product of a wild type allele and/or of a mutant allele of a gene name ClD14 (Citrullus lanatus Dwarf14), comprising:
a) providing genomic DNA of a watermelon plant or of a plurality of plants (e.g. a breeding population, F2, backcross, etc.).
b) carrying out an assay (e.g. a bi-allelic genotyping assay) that discriminates or can discriminate between the presence of alleles in the genomic DNA of a), which assay generates a nucleic acid amplification product (e.g. through the use of allele specific oligonucleotide primers to generate the product) and/or which assay generates a nucleic acid hybridization product (e.g. through the use of allele-specific oligonucleotide probes to generate the hybridization product), whereby the amplification product or hybridization product indicates the presence of a wild type allele of the gene and/or a mutant allele of the gene in the DNA, wherein the wild type allele comprises the sequence of SEQ ID NO: 6 (or wherein the wild type allele encodes the protein of SEQ ID NO: 2) and the mutant allele comprises one or more nucleotides inserted, duplicated, deleted or replaced with respect to the sequence of SEQ ID NO: 6 (or the mutant allele encodes a protein comprising one or more amino acids inserted, duplicated, deleted or replaced with respect to the wild type protein of SEQ ID NO: 2), and optionally
c) selecting a plant, seed or plant part comprising one or two copies of the mutant allele.

Also a method of amplifying all or part of a mutant and/or wild type ClD14 allele from a genomic DNA sample derived from a watermelon plant, plant part or seed is provided, comprising contacting genomic DNA with a primer pair which amplifies all or part of the mutant ClD14 or wild type ClD14 allele in the sample, and detecting the amplification product.

Also, a method of hybridizing a probe to a mutant and/or wild type ClD14 allele in a genomic DNA sample derived from a watermelon plant, plant part or seed is provided, comprising contacting genomic DNA with a oligonucleotide probe which hybridizes to the mutant ClD14 or wild type ClD14 allele in the sample, and detecting the hybridization product.

All embodiments described above and elsewhere herein also apply to these embodiments. The amplification product may thus be a PCR amplification product. e.g. competitive PCR amplification product generated in e.g. a KASP assay or other assay, to detect the mutant and/or wild type allele in the DNA sample. The hybridization product may thus be a hybridization product of an oligonucleotide probe which hybridizes to the nucleic acid in the DNA sample, to detect the mutant and/or wild type allele in the DNA sample. The primer pairs or probes preferably are allele specific, and the products are thus distinguishable as being either two copies of the wild type allele, two copies of the mutant allele or one copy of each being present in the genomic DNA of the watermelon plant, plant part or seed.

The primers or probes are preferably modified, e.g. labeled by a tail sequence or fluorescent label or otherwise modified with respect to the wild type sequence which they amplify or hybridize.

As the described methods require detection of a mutant and/or wild type allele in the genomic DNA of the plant, plant part or seed, the genomic DNA needs to be accessible for detection, e.g. it may be extracted from the plant cells using DNA extraction methods or at least eluted from the damaged cells into a solution (e.g. a buffer solution).

As the ortholog genes in other Cucurbitaceae are provided herein, the above methods can also be applied to other D14 genes and alleles in other species, especially cucumber and melon.

In one aspect, therefore, a genotyping assay is provided for genotyping watermelon, cucumber or melon plants, seeds, plant parts, cells or tissues, comprising the steps:
a) providing genomic DNA of one or more watermelon, cucumber or melon plants or a population of plants (e.g. breeding population. F2 population, backcross population etc.), and
b) carrying out a genotyping assay which is able to detect, or which detects, the presence of the wild type allele of SEQ ID NO: 6 or comprising at least 95% identity thereto (watermelon gene) or SEQ ID NO: 15, or comprising at least 95% identity thereto (cucumber gene) or SEQ ID NO: 16 or comprising at least 95% identity thereto (melon gene), and/or the presence of a mutant allele, wherein the mutant allele comprises one or more nucleotides inserted, deleted, replaced or duplicated with respect of SEQ ID NO: 6 (or with respect to the wild type sequence comprising at least 95% identity thereto), SEQ ID NO: 15 (or with respect to the wild type sequence comprising at least 95% identity thereto) or SEQ ID NO: 16 (or with respect to the wild type sequence comprising at least 95% identity thereto), and optionally
c) selecting a plant, seed, plant part, cell or tissue comprising either two copies of the wild type allele, or one copy of the wild type allele and one copy of a mutant allele, or two copies of a mutant allele.

In one aspect, a genotyping assay genotyping watermelon, melon or cucumber plants, seed, plant parts, cells or tissues, comprising the steps is provided, comprising the steps:
a) providing genomic DNA of one or more watermelon, cucumber or melon plants or a population of plants (e.g. breeding population, F2 population, backcross population etc.), and
b) carrying out a genotyping assay which is able to detect (or which detects) the presence of the wild type allele encoding the protein of SEQ ID NO: 2 or a protein comprising at least 95% sequence identity thereto (watermelon wild type ClD14 protein) or SEQ ID NO 8 or a protein comprising at least 95% sequence identity thereto (cucumber wild type ClD14 protein) or SEQ ID NO: 9 or a protein comprising at least 95% sequence identity thereto (melon wild type ClD14 protein) and/or the presence of a mutant allele, wherein the mutant allele comprises one or more amino acids inserted, deleted, replaced or duplicated with respect of SEQ ID NO: 2 (or with respect to the wild type sequence comprising at least 95% identity thereto), or SEQ ID NO: 8 (or with respect to the wild type sequence comprising at least 95% identity thereto) or SEQ ID NO: 9 (or with respect to the wild type sequence comprising at least 95% identity thereto), and optionally
c) selecting a plant, seed, plant part, cell or tissue comprising either two copies of the wild type allele, or one copy of the wild type allele and one copy of a mutant allele, or two copies of a mutant allele.

Thus, a method is provided for detecting, and optionally selecting, a watermelon, cucumber or melon plant, seed or plant part comprising at least one copy of a wild type allele and/or of a mutant allele of a gene name ClD14 (Citrullus lanatus Dwarf14), CsD14 (Cucumis sativus Dwarf14) or CmD14 (Cucumis melo Dwarf14) comprising:
a) carrying out an assay on a genomic DNA sample obtained from at least one plant that detects or discriminates between D14 alleles based on nucleic acid amplification and/or nucleic acid hybridization to detect the presence of a wild type allele of the gene and/or a mutant allele of the gene, wherein the wild type allele encodes the protein of SEQ ID NO: 2 or a protein comprising at least 95% sequence identity thereto (in watermelon), SEQ ID NO: 8 or a protein comprising at least 95% sequence identity thereto (in cucumber) and SEQ ID NO: 9 or a protein comprising at least 95% sequence identity thereto (in melon) and the mutant allele comprises one or more amino acids inserted, deleted or replaced with respect to SEQ ID NO: 2 (or with respect to the wild type sequence comprising at least 95% identity thereto), SEQ HD NO: 8 (or with respect to the wild type sequence comprising at least 95% identity thereto) or SEQ ID NO: 9 (or with respect to the wild type sequence comprising at least 95% identity thereto), and optionally
b) selecting a plant, seed or plant part comprising one or two copies of the mutant allele.

Further a method is provided for determining the genotype of the D14 gene, and optionally selecting, a watermelon, cucumber or melon plant, seed or plant part comprising certain genotype, e.g. at least one copy of a wild type allele and/or of a mutant allele of a gene name ClD14 (Citrullus lanatus Dwarf14), CsD14 (Cucumis sativus Dwarf14) or CmD14 (Cucumis melo Dwarf14) comprising:
a) carrying out a bi-allelic genotyping assay on one or more genomic DNA samples, obtained from one or more plants, wherein said genotyping assay detects or discriminates between D14 alleles based on D14 allele-specific primers and/or D14 allele-specific probes which allele specific primers or allele specific probes detect the presence of a wild type allele of the gene or of a mutant allele of the gene, wherein the wild type allele encodes the protein of SEQ ID NO: 2 or a protein comprising at least 95% sequence identity thereto (in watermelon), SEQ ID NO: 8 or a protein comprising at least 95% sequence identity thereto (in cucumber) and SEQ ID NO: 9 or a protein comprising at least 95% sequence identity thereto (in melon) and the mutant allele comprises one or more amino acids inserted, deleted or replaced with respect to SEQ ID NO: 2 (or with respect to the wild type sequence comprising at least 95% identity thereto), SEQ ID NO: 8 (or with respect to the wild type sequence comprising at least 95% identity thereto) or SEQ ID NO: 9 (or with respect to the wild type sequence comprising at least 95% identity thereto), and optionally
b) selecting one or more plants, seeds or plant parts comprising one or two copies of the mutant allele.

Such an assay can be used for marker assisted selection (MAS) of plants in e.g. a breeding program to select plants comprising a certain genotype, e.g. homozygous for the wild type allele of the D14 gene (having normal secondary branching), homozygous or heterozygous for a mutant allele of the D14 allele.

Therefore, also a method of breeding watermelon, cucumber or melon plants is provided herein, said method comprising genotyping one or more plants for the allele composition at the D14 locus in the genome and optionally selecting one or more plants having a specific genotype at the D14 locus. In one aspect also genotyping-by-sequencing may be done for the D14 gene.

As mentioned, optionally the plants or seeds which comprise two copies of a mutant D14 allele can be grown and phenotyped for the secondary branching phenotype. The mutant allele is in one aspect a mutant allele which, in homozygous form, confers multibranching/increased secondary branching. In one aspect the mutant allele confers ‘full multibranching’ when in homozygous form. In another aspect the mutant allele confers ‘intermediate multibranching’ when in homozygous form. Thus, the mutant allele may comprise one or more nucleotides replaced, inserted or deleted, whereby the encoded protein has a loss-of-function or whereby the allele is not expressed in the plant, leading to ‘full multibranching’ when the mutant allele is in homozygous form, or the mutant allele may comprise one or more nucleotides replaced, inserted or deleted, whereby the encoded protein has a reduced-function or whereby the allele has a reduced expression in the plant, leading to ‘intermediate multibranching’ when the mutant allele is in homozygous form.

In one aspect the mutant allele encodes a protein having reduced function or a loss-of-function in vivo due to the protein comprising at least one amino acid of the 1PR000073 domain of SEQ ID NO: 2, SEQ ID NO: 8 and SEQ ID NO: 9 (or the equivalent amino acids in a protein comprising at least 95% identity to any of these) being deleted, or being replaced by different amino acid or by a stop codon. In another aspect the mutant allele encodes a protein having reduced function or a loss-of-function in vivo due to the protein comprising at least one amino acid being inserted or duplicated in the IPR000073 domain of SEQ ID NO: 2, SEQ ID NO: 8 and SEQ ID NO: 9 (or the equivalent amino acids in a protein comprising at least 95% identity to any of these).

In a different aspect the mutant allele encodes a protein having reduced function or a loss-of-function in vivo due to the protein comprising at least one amino acid of the helical lid domain of SEQ ID NO: 2, SEQ ID NO: 8 and SEQ ID NO: 9 (or the equivalent amino acids in a protein comprising at least 95% identity to any of these) being deleted, or being replaced by different amino acid or by a stop codon. In another aspect the mutant allele encodes a protein having reduced function or a loss-of-function in vivo due to the protein comprising at least one amino acid being inserted or duplicated in the helical lid domain of domain of SEQ ID NO: 2, SEQ ID NO 8 and SEQ ID NO: 9 (or the equivalent amino acids in a protein comprising at least 95% identity to any of these).

In another aspect the mutant allele encodes a protein having reduced function or a loss-of-function in vivo due to the protein comprising at least one amino acid of the catalytic triad of SEQ ID NO: 2, SEQ ID NO: 8 and SEQ ID NO: 9 (or the equivalent amino acids in a protein comprising at least 95% identity to any of these), or of the 1, 2, 3, 4, 5, 6, 7, or 8 amino acids preceding or following a catalytic triad amino acid, being deleted, or being replaced by different amino acid or by a stop codon. In another aspect the mutant allele encodes a protein having reduced function or a loss-of-function in vivo due to the protein comprising at least one amino acid of the catalytic triad of SEQ ID NO: 2, SEQ ID NO: 8 and SEQ ID NO: 9 (or the equivalent amino acids in a protein comprising at least 95% identity to any of these) being duplicated, or at least one of the 1, 2, 3, 4, 5, 6, 7, or 8 amino acids preceding or following a catalytic triad amino acid, being duplicated, or at least one amino acid being inserted into the stretch of 8 amino acids preceding or following a catalytic triad amino acid.

In yet another aspect the mutant allele encodes a protein of Table A or Table 2.

In one aspect the mutant allele encodes a protein comprising a duplication of at least one amino acid selected from amino acids 94 to 101 of SEQ ID NO: 2, SEQ ID NO: 8 and SEQ ID NO: 9 (or the equivalent amino acids in a protein comprising at least 95% identity to any of these).

In one aspect the mutant allele encodes a protein comprising a duplication of at least Serine 97 of SEQ ID NO: 2, SEQ ID NO: 8 and SEQ ID NO: 9 (or the equivalent amino acid in a protein comprising at least 95% identity to any of these).

In yet a further aspect the mutant allele encodes a protein comprising a duplication of amino acids 94 to 101 of SEQ ID NO: 2, SEQ ID NO: 8 or SEQ ID NO: 9 (or the equivalent amino acids in a protein comprising at least 95% identity to any of these).

The aspects described further above for assays for detecting the watermelon ClD14 wild type and/or mutant alleles apply also to assays for detecting cucumber CsD14 wild type and/or mutant alleles or melon CmD14 wild type and/or mutant alleles.

In a different aspect a watermelon, cucumber or melon plant, seed or plant part is provided comprising at least one copy of a mutant allele of a gene name ClD14 in watermelon, CsD14 in cucumber and CmD14 in melon, wherein said mutant allele either
a) comprises one or more mutations in a regulatory element, resulting in no expression or reduced expression of the allele compared to the wild type allele, and/or
b) encodes a mutant protein comprising one or more amino acids replaced, inserted, duplicated or deleted compared to the wild type protein,

wherein said mutant allele of a) or b) confers an increased average number of secondary branches developing when the mutant allele is in homozygous form (compared to the plant comprising the wild type allele in homozygous form), and wherein the wild type watermelon ClD14 allele encodes a protein of SEQ ID NO: 2 or a protein comprising at least 95%, 96%, 97%, 98%, 99% or more sequence identity to SEQ ID NO: 2, wherein the wild type cucumber CsD14 allele encodes a protein of SEQ ID NO: 8 or a protein comprising at least 95%, 96%, 97%, 98%, 99% or more sequence identity to SEQ ID NO: 8, wherein the wild type melon CmD14 allele encodes a protein of SEQ ID NO: 9 or a protein comprising at least 95%, 96%, 97%, 98%, 99% or more sequence identity to SEQ ID NO: 9.

The wild type functional D14 protein of watermelon is provided in SEQ ID NO: 2, of cucumber in SEQ ID NO: 8 and of melon in SEQ ID NO: 9. There may however be some amino acid sequence variation within watermelons, cucumbers and melons and functional D14 proteins may comprise e.g. at least 1, 2, 3, 4, 5, 6, 7, 8, 9, 10, 11, 12, 13, 14, 15 or more amino acids which are different than in SEQ ID NO: 2. SEQ ID NO: 8 or SEQ ID NO:9 provided herein or whereby the protein comprises comprising at least 95%, 96%, 97%, 98%, 99% or 99.3%, 99.4%, 99.5% or 99.6%, 99.7%, 99.8% or 99.9% sequence identity to the proteins of SEQ ID NO: 2, 8 or 9 (when aligned pairwise using e.g. Emboss-Needle). Such functional variants of the D14 protein of SEQ ID NO: 2, 8 or 9 may exist in other lines or varieties. These alleles may, thus, vary in sequence, but the phenotype of the plant is equal to the wild type phenotype. Such functional variant alleles (allelic variants) can be found by e.g. sequencing the D14 gene of many different watermelon, cucumber or melon lines or varieties which have a normal secondary branching pattern.

Therefore, in one aspect functional variants of the proteins of SEQ ID NO: 2, 8 or 9 are proteins comprising at least 90%, 91%, 92%, 93%, 94%, 95%, 9%%, 97%, 98%, 99% or 99.3%, 99.4%, 99.5% or 99.6%, 99.7%, 99.8% or 99.9% sequence identity to the protein of SEQ ID NO: 2, 8 or 9, when aligned pairwise (using e.g. Needle with default parameters).

In one aspect a watermelon, cucumber or melon plant, seed or plant part is provided comprising at least one copy of a mutant allele of a gene name D14, wherein said mutant allele encodes a mutant protein comprising one or more amino acids inserted, duplicated, deleted or replaced in the region of the protein selected from:
a) the region starting at amino acid 94 and ending at amino acid 101 of SEQ ID NO: 2, 8 or 9, or the equivalent amino acids in a variant D14 protein comprising at least 95% sequence identity to SEQ ID NO: 2, 8 or 9.
b) the region of the IPR000073 domain starting at amino acid 22 and ending at amino acid 259 of SEQ ID NO: 2, 8 or 9, or the equivalent amino acids in a variant D14 protein comprising at least 95% sequence identity to SEQ ID NO: 2, 8 or 9,
c) the region of the helical lid domain starting at amino acid 136 and ending at amino acid 193 of SEQ ID NO: 2, 8 or 9, or the equivalent amino acids in a variant D14 protein comprising at least 95% sequence identity to SEQ ID NO: 2, 8 or 9,
d) the catalytic triad amino acids or the 1, 2, 3, 4, 5, 6, 7, or 8 amino acids preceding or following the catalytic triad amino acids S97, D218 and 11247 of SEQ ID NO: 2, 8 or 9, or the equivalent amino acids in a variant D14 protein comprising at least 95% sequence identity to SEQ ID NO: 2, 8 or 9,

and wherein said mutant allele confers a (significantly) increased average number of secondary branches developing when the mutant allele is in homozygous form, preferably an intermediate multibranching or full multibranching phenotype when the mutant allele is in homozygous form.

The term ‘starting at’ and ‘ending at’ or ‘from’ and ‘to’ includes the first and last amino acid mentioned.

Under a) the insertion, duplication, deletion and/or replacement of one or more amino acids in the in the region of the protein starting at amino acid 94 and ending at amino acid 101 of SEQ ID NO: 2, 8 or 9, may be the insertion, duplication, deletion and/or replacement of at least 1, 2, 3, 4, 5, 6, 7 or 8 amino acids, preferably of at least S97.

In one aspect at least 1, 2, 3, 4, 5, 6, 7 or 8 consecutive amino acids of amino acids 94 to 101 are duplicated, deleted or replaced, preferably including at least a duplication, deletion or replacement of S97. In one aspect the mutant allele comprises a duplication or deletion or replacement of H9 (Histidine 96) and S97 (Serine 97); or of S97 (Serine 97) and V98 (Valine 98); or of H96 (Histidine 96), S97 (Serine 97) and V98 (Valine 98); or of G95 (Glycine 95), H96 (Histidine %), S97 (Serine 97), V98 (Valine 98) and S99 (Serine 99); or of V94 (Valine 94), G95 (Glycine 95), H96 (Histidine 96), S97 (Serine 97), V98 (Valine 98), S99 (Serine 99) and A100 (Alanine 100); or of V94 (Valine 94), G95 (Glycine 95), H % (Histidine 96). S97 (Serine 97), V98 (Valine 98), S99 (Serine 99), A100 (Alanine 100) and M101 (Methionine 101).

In another aspect a watermelon, cucumber or melon plant, seed or plant part is provided comprising at least one copy of a mutant allele of a gene name D14, wherein said mutant allele encodes a mutant protein comprising one or more amino acids inserted, duplicated, deleted or replaced in the region of the protein starting at amino acid 197 and ending at amino acid 249 of SEQ ID NO: 2, 8 or 9, or the equivalent amino acids in a variant D14 protein comprising at least 95% sequence identity to SEQ ID NO: 2, 8 or 9, and wherein said mutant allele confers an increased average number of secondary branches developing when the mutant allele is in homozygous form. Thus, one aspect is an insertion, duplication, deletion and/or replacement of one or more amino acids in the in the region of the protein starting at amino acid 197 and ending at amino acid 249 of SEQ ID NO: 2, 8 or 9, may be the insertion, duplication, deletion and/or replacement of at least 1, 2, 3, 4, 5, 6, 7 or 8 amino acids, preferably of at least D218 or 1H247.

In yet another aspect a watermelon, cucumber or melon plant, seed or plant part is provided comprising at least one copy of a mutant allele of a gene name D14, wherein said mutant allele encodes a mutant protein comprising at least 4, 5, 6, 7, 8, 9, 10, 20, 30, 40, 50, 60, 70, 80, 90, 100, 110, 120, 130, 140, 150, 160, 170, 180, 190, 200 or more amino acids inserted, duplicated, deleted and/or replaced in SEQ ID NO: 2, 8 or 9 or in a variant D14 protein or a protein comprising at least 95% sequence identity to SEQ ID NO: 2, 8 or 9, and wherein said mutant allele confers the modified phenotype described when the mutant allele is in homozygous form. The mutant D14 protein may thus e.g. be truncated at the N-terminal or C-terminal, lacking said at least 4, 5, 6, 7, 8, 9, 10, 20, 30, 40, 50, 60 70, 80, 90, 100, 150, 200 more amino acids at the N-terminal or C-terminal, or any other at least 4, 5, 6, 7, 8, 9, 10 amino acids may be deleted, replaced or inserted or duplicated compared to the wild type functional D14 protein. In one aspect at least 1, 2, 3, 4, 5, 6, 7 or 8 amino acids (preferably consecutive amino acids) are deleted, duplicated or replaced, whereby the deletion, duplication or replacement includes an amino acid of the catalytic triad, selected from S97, D218 and H247 of SEQ ID NO: 2, 8 or 9, or the equivalent amino acid in a variant sequence of any of these.

Mutant alleles can be generated by various techniques, such as random mutagenesis or targeted gene editing, and the phenotype of the mutant allele can then be analysed in plants homozygous for the mutant allele. Using random or targeted mutagenesis techniques any mutation can be generated or reconstructed, e.g. the mutants described herein can be easily made de novo. TILLING primers can for example be designed to the specific mutations in the allele, enabling de novo identification of e.g. M2 plants comprising the mutants described herein. The mutant allele present in variety Sidekick F1 can also be made de novo. No seed deposit is requirement for enablement when the gene sequence is disclosed. Similarly, targeted gene editing can be used to generate any desired mutation in the allele.

TILLING is described e g in McCallum, et al. (June 2000). “Targeting induced local lesions IN genomes (TILLING) for plant functional genomics”. Plant Physiol. 123 (2): 439-42.

In one aspect the mutant allele of the ClD14 gene is not the mutant allele present in variety Sidekick F1 but a different mutant allele, e.g. one or more nucleotides may be different, but e.g. due to the degeneracy of the genetic code the mutant protein encoded may still be the same (i.e. the protein of SEQ ID NO: 1), or one or more amino acids may be different compared to SEQ ID NO: 1 (i.e. pairwise alignment of the mutant proteins does not give 100% sequence identity to SEQ ID NO: 1). For example, instead of a duplication of 8 amino acids, only 5, 6 or 7 amino acids may be duplicated; or 9, 10 or 11 amino acids may be duplicated. In another aspect the mutant allele of the ClD14 gene is identical to the mutant allele present in variety Sidekick F1, but is induced de novo by mutagenesis techniques, such as CRISPR based techniques.

Any mutant allele in the ClD14. CsD14 or CmD14 gene which results in an insertion, deletion and/or replacement of one or more amino acids of the wild type, functional protein may result in a mutant protein having reduced function or no function and may, thus, result in the phenotype of significantly more secondary branches developing when the mutant allele is in homozygous form. Plants and plant parts comprising such mutant alleles are one embodiment herein.

The ‘equivalent amino acid’ can easily be determined by pairwise amino acid sequence alignment, using e.g. Emboss Needle (default parameters).

In one aspect the mutant allele encodes a protein comprising a duplication or insertion of the codons of amino acid number S97, D218 or 11247 of SEQ ID NO: 2, 8 or 9, or the equivalent amino acid in a protein comprising at least 95%, 96%, 97%, 98% or 99% sequence identity to SEQ ID NO: 2, 8 or 9.

In one aspect the mutant allele encodes a protein comprising a duplication or insertion of the codons of one or more of the amino acid number 94 to 101 of SEQ ID NO: 2, 8 or 9, or the equivalent amino acids in a protein comprising at least 95%, 96%, 97%, 98% or 99% sequence identity to SEQ ID NO: 2, 8 or 9.

A mutation in a codon may be a (at least one) nucleotide insertion, deletion or replacement in the codon, leading to e.g. a different reading frame or a different codon, e.g. encoding a different amino acid or a STOP codon. Also the entire codon may be deleted or replaced by a different codon (or optionally a stop codon), resulting in either a deletion of the encoded amino acid, or the replacement thereof.

In one aspect the mutant allele encodes a protein comprising an amino acid substitution (replacement) or deletion or a stop codon of amino acid number S97, D218 or H247 of SEQ ID NO: 2, 8 or 9, or the equivalent amino acid in a protein comprising at least 95%, 96%, 97%, 98% or 99% sequence identity to SEQ ID NO: 2, 8 or 9.

In one aspect the mutant allele encodes a mutant ClD14, CsD14 or CmD14 protein which comprises a truncation of at least 10, 20, 30, 40, 50, 60, 70, 80, 90, 100, 110, 113, 115, 120, 130, 140, 150, 160, 170, 180, 190 or 200 amino acids of the C-terminal end of the protein of SEQ ID NO: 2, 8 or 9 or of the C-terminal end of a protein comprising at least 95%, 96%, 97%, 98% or 99% sequence identity to SEQ ID NO: 2, 8 or 9. In one aspect all amino acids starting at (and including) amino acid 94, 95, 96 or 97, or starting at (and including) amino acid 218, or starting at (and including) amino acid 247 of SEQ ID NO: 2, 8 or 9, or the equivalent amino acid in a protein comprising at least 95%, 96%, 97%, 98% or 99% sequence identity to SEQ ID NO: 2, 8 or 9, are deleted or replaced by one or more different amino acids.

As mentioned the watermelon, cucumber or melon plant or plant part may comprise a mutant D14 allele, wherein the mutant allele is produced by random mutagenesis or targeted mutagenesis, such as CRISPR based methods. Random mutagenesis may for example be chemical induced (e.g. EMS treatment) or radiation induced mutagenesis or other methods, whereby mutations are randomly induced in the genome and then plants or plant parts comprising mutations in the endogenous D14 gene can be screened for and identified. Targeted mutagenesis are methods whereby mutations are specifically introduced into a target gene, such as the D14 gene, using e.g. Crispr-Cas9, or Crispr-Cpf1 or other known methods.

In one aspect the plant comprising the mutant allele is not produced exclusively by an essentially biological process, meaning that the mutant allele has at one point been generated by human intervention. If such a human generated mutant allele is transferred from one plant to another by crossing and selection, then the patent covers plants comprising the mutant allele, even if the plant itself has been generated solely by crossing and selection.

In one aspect the watermelon, cucumber or melon plant is diploid and comprises at least one copy of a mutant D14 allele as described above, i.e. the plant is heterozygous. As the phenotype is only seen when the mutant allele is in homozygous form, these plants have normal secondary branching. Selfing of such heterozygous plants will generate a plant which is homozygous and which comprises two copies of the mutant allele. In one aspect the watermelon plant is diploid and comprises two copies of a mutant D14 allele as described above, i.e. the plant is homozygous. The plant therefore also has a modified phenotype as described herein.

The plants and plant parts comprising at least one copy of a mutant D14 allele is preferably a cultivated plant, not a wild plant. So preferably cultivated watermelon (Citrullus lanatus), cucumber or melon. The plant may be an inbred line, a F1 hybrid or a breeding line.

In one aspect the plant is a watermelon plant and the watermelon plant is diploid, triploid or tetraploid, comprising at least one copy of a mutant ClD14 allele. The diploid plant or plant part comprises in one aspect two copies, the triploid plant or plant part comprises one, two or three copies and the tetraploid plant or plant part comprises two or four copies of the mutant ClD14 allele. It is noted that the genotyping methods or assays described herein for diploid plants, seeds and plant parts are equally applicable to triploid or tetraploid plants, seeds or tissues/plant parts. One can select triploid plants, seeds or parts comprising 1, 2 or 3 copies of a mutant ClD14 allele or of a wild type allele, and one can select tetraploid plants comprising 1, 2, 3 or 4 copies of a mutant ClD14 allele or of a wild type allele. A KASP assay, for example, can be used to analyze triploids and tetraploid genomic DNA for the ClD14 alleles present and their copy number.

Also seeds from which a plant or plant part as described above can be grown are encompassed herein.

The plant part comprising at least one copy of the mutant D14 allele may be a cell, a flower, a leaf, a stem, a cutting, an ovule, pollen, a root, a rootstock, a scion, a fruit, a protoplast, an embryo, an anther.

Further a vegetatively propagated plant propagated from a plant part and comprising at least one copy of a mutant D14 allele in its genome is provided.

In one aspect also a method of producing diploid, seeded watermelon fruits is provided, said method comprising growing a diploid watermelon plant comprising one or two copies of a mutant ClD14 allele, allowing pollination of the flowers and optionally harvesting the diploid, seeded fruits that develop on the plant, whereby the fruit tissue also comprises one or two copies of the mutant ClD14 allele.

In one aspect also a method of producing seedless watermelon fruits is provided, said method comprising growing a diploid watermelon plant comprising two copies of a mutant ClD14 allele in the vicinity of a triploid watermelon plant, allowing pollination of the flowers of the triploid plant with pollen of the diploid plant and optionally harvesting the seedless fruits that develop on the triploid plant and/or the seeded fruits that develop on the diploid plant following self pollination of the diploid plant.

When referring to ‘growing in the vicinity’ this means that the diploid pollenizer plants are near enough to the triploid plants to allow insects, who can visit the pollenizer plants, to transfer the pollen from the male flowers of the pollenizer plant to the triploid plants. The pollenizer may be interplanted in rows or in between rows or randomly in the same field as the triploid plants. Also, the pollenizer may be grafted to the same rootstock as a triploid plant, to generate a double grafted plant. Such double grafted plants can then be grown in the vicinity of triploid plants, in order to provide pollen to those plants.

In one aspect the mutant ClD14 allele may be combined with a different genes, such as the Ts-gene (tomato-seed size gene) on chromosome 2, as described in WO2021/165091. By combining the mutant ClD14 alleles described herein and the Ts-gene deletion or mutant alleles encoding e.g. a reduced function or loss of function Ts-protein, the plants having a ‘strong multibranching’ or ‘intermediate multibranching’ phenotype, as described herein, can produce fruits having small seeds. As seed size is determined by chromosome 2 and chromosome 6 loci (as described in WO2021/165091), the fruits of plants described herein can actually have any seed size, from large to intermediate to very small.

However, the ClD14 alleles described herein may also be combined with genes conferring parthenocarpy or stenospermocarpy, so that seedless fruits may be produced on the plants having a ‘strong multibranching’ or ‘intermediate multibranching’ phenotype, as described herein. See WO2022/096451, WO2022/078792, WO2019238832, WO2018060444 or WO2017202715, all incorporated herein by reference.

Also provided is a method for screening plants, plant parts, or DNA therefrom, for the presence of a mutant allele of a gene named ClD14, CsD14 or CmD14, or for selecting a plant or plant part comprising a mutant allele of a gene named ClD14, CsD14 or CmD14, or for generating a plant or plant part comprising a mutant allele of a gene named ClD14, CsD14 or CmD14, wherein said mutant allele either
a) comprises one or more mutations in a regulatory element, resulting in no expression or reduced expression of the allele compared to the wild type allele, and/or
b) encodes a mutant protein comprising one or more amino acids replaced, inserted, duplicated and/or deleted compared to the wild type protein,

wherein the wild type watermelon allele encodes a protein of SEQ ID NO: 2 or a protein comprising at least 95% sequence identity to SEQ ID NO: 2, wherein the wild type cucumber allele encodes a protein of SEQ ID NO: 8 or a protein comprising at least 95% sequence identity to SEQ ID NO: 8, wherein the wild type melon allele encodes a protein of SEQ ID NO: 9 or a protein comprising at least 95% sequence identity to SEQ ID NO: 9.

As mentioned before, the methods herein preferably relate to the mutant alleles described herein, which, when in homozygous form, result in ‘full multibranching’ or ‘intermediate multibranching’ phenotypes of the plant.

A method for screening plants, plant parts or DNA therefrom involves providing genomic DNA or sequence information of genomic DNA, determining the D14 gene sequence in the genomic DNA and comparing the gene sequence to the wild type gene sequence, or genotyping the genomic DNA for the alleles at the D14 locus, e.g. amplifying all or part of the gene sequence or cDNA (mRNA) using e.g. PCR primers, or sequencing the genomic region (e.g. genotyping by sequencing) and comparing the D14 allele sequences to the wild type sequences.

A method for generating mutants comprises e.g. mutagenizing one or mom seeds, plants or plant parts of watermelon, cucumber or melon (using e.g. radiation or chemical mutagenic agents), or providing a population of mutagenized plants, and screening the M1 or M2 or further generation for mutant D14 alleles being present. A plant comprising a mutant allele can then be made homozygous for the mutant allele to analyze the phenotype.

In one aspect the mutant ClD14, CsD14 or CmD14 allele comprises a mutation in the genomic DNA, resulting in the expression of a mutant D14 protein comprising one or more amino acids inserted, duplicated, deleted or replaced as described elsewhere herein, e.g. a duplication of amino acids 94 to 101 of SEQ ID NO: 2, 8 or 9 (or the equivalent amino acid in a sequence comprising at least 95% identity to SEQ ID NO: 2, 8 or 9).

Thus, any mutant allele of the ClD14, CsD14 or CmD14 gene, causing at least a (significantly) higher average number of secondary branches to develop when in homozygous form, are embodiments of the invention. Such mutant ClD14, CsD14 or CmD14 alleles can be generated by the skilled person without undue burden. The skilled person can, for example, generate mutants in the ClD14, CsD14 or CmD14 gene and determine whether they result in at least a higher average number of secondary branches when in homozygous form in a diploid plant, compared to e.g. the diploid plant which is homozygous for the wild type allele. The skilled person can also generate mutants which encode a non-functional protein, which plants can e.g. serve as a comparison.

Thereby new mutants can be compared to the wild type branching phenotype and the ‘full multibranching’ phenotype, in order to determine if the effect of the mutant allele is e.g. ‘intermediate multibranching’ or ‘full multibranching’. It is preferred to compare the phenotype of the mutant alleles in the same genetic background line, so e.g. in the non-mutagenized line (control, wild type) and preferably also a mutant line having the full multibranching phenotype in the same background line. That way the lowest branching and the strongest branching phenotypes are in the same background and any new mutants can be compared and positioned in the broadest range.

Having identified the nucleotide sequence of the gene, the skilled person can generate watermelon, cucumber or melon plants comprising mutants in the D14 gene by various methods, e.g. mutagenesis, TILLING or CRISPR-Cas or other methods known in the art. Especially with targeted gene modification technologies such as Crispr-Cas, TALENS and others, targeted mutations can be made by the person skilled in the art. The skilled person can then confirm the phenotype of a plant homozygous for the mutant D14 allele, i.e. developing a higher average number of secondary branches. Therefore, the skilled person is not limited to the specific D14 mutants disclosed herein, but the skilled person can equally generate other mutations in the D14 allele of watermelon, cucumber or melon and thereby generate other mutants which lead to multibranching when in homozygous form. Various mutations can be generated and tested for the resulting phenotype, for example the regulatory elements can be mutated to reduce expression (knock-down) or eliminate expression (knock-out) of the allele and thus reduce or eliminate the amount of wild type D14 protein present in the cell or plant. Alternatively, mutations which lead to reduced function or loss-of-function of the D14 protein can be generated, i.e. mutations (such as missense mutations or frame shift mutations) which lead to one or more amino acids being substituted, inserted, duplicated and/or deleted, or whereby the protein is truncated through the introduction of a premature stop-codon in the coding sequence (non-sense mutations).

As the D14 protein comprises conserved amino acids of the catalytic triad, it is in one aspect encompassed that one or more amino acids of the catalytic triad, or comprising an amino acid of the catalytic triad, are replaced, deleted, duplicated and/or inserted, as such mutations will likely result in a loss of function.

Whether any mutation in a D14 allele results in the expected phenotype can then be tested by generating plants homozygous for the mutation and growing the plant line next to a wild type plant line and analysing the phenotypes of both lines, e.g. the multibranching phenotype.

Alternatively, the skilled person can carry out a method for production of a cultivated watermelon, cucumber or melon plant capable of producing a higher average number of secondary branches (multibranching) and/or a method for generating watermelon, cucumber or melon plants comprising mutant D14 alleles comprising the steps of:
a) introducing mutations in a population of watermelon, cucumber or melon plants, plant parts or seeds, especially cultivated plants, or providing a population of mutated plants or progeny thereof;
b) selecting a plant developing, when grown, a higher average number of secondary branches;
c) optionally determining if the plant selected under b) comprises a mutant allele of a D14 gene; and
d) optionally growing the plants obtained under c).

Steps b) and c) can also be switched, so that step b) is selecting a plant comprising a mutant allele of a D14 gene and step c) is optionally determining if the plant (or a progeny thereof) produce a higher average secondary branching/multibranching phenotype.

Step a) can be carried out by e.g. mutagenizing seeds of one or more lines or varieties of watermelon, cucumber or melon, for example by treatment with mutagenizing agents such as chemical mutagens, e.g. EMS (ethyl methane sulphonate), or irradiation with UV radiation, X-rays or gamma rays or the like. The population may for example be a TILLING population. Preferably the mutagenized plant population is selfed at least once (e.g. to produce an M2 generation, or M3, M4, etc.) prior to carrying out step b).

The phenotyping of step b) can be easily done visually, e.g. by counting the secondary branches.

Such plants, or progeny thereof, can be tested for the presence of the mutant D14 gene by phenotypic analysis (e g secondary branching) and/or by genotyping the plants for mutations in the D14 gene and encoded protein, or expression of the D14 gene, sequencing and other methods known to the skilled person. There are, thus, various methods, or combinations of methods, for verifying if a phenotypically selected plant comprises a mutant allele of a D14 gene.

If step b) is the selection of plants comprising a mutant allele of the D14 gene, the skilled person can also use various methods for detecting the DNA, mRNA or protein of the D14 gene in order to identify a plant comprising a mutant D14 allele. The genomic DNA of the wild type watermelon ClD14 gene, encoding a functional ClD14 protein (SEQ ID NO: 2) is the DNA of SEQ ID NO: 6 and the cDNA (mRNA) encoding the protein of SEQ ID NO: 2 is given in SEQ ID NO: 4. The promoter is upstream of this sequence and can e.g. be retrieved by sequencing or from the watermelon genome database. For example, the at least 1000 or at least 2000 bases upstream of the ATG start include the promoter sequence.

The genomic DNA of the wild type cucumber CsD14 gene, encoding a functional CsD14 protein (SEQ ID NO: 8) is the DNA of SEQ ID NO: 15 and the cDNA (mRNA) encoding the protein of SEQ ID NO: 8 is given in SEQ ID NO: 17. The promoter is upstream of this sequence and can e.g. be retrieved by sequencing or from the cucumber genome database. For example, the at least 1000 or at least 2000 bases upstream of the ATG start include the promoter sequence.

The genomic DNA of the wild type melon CmD14 gene, encoding a functional CmD14 protein (SEQ ID NO 9) is the DNA of SEQ ID NO: 16 and the cDNA (mRNA) encoding the protein of SEQ ID NO 9 is given in SEQ ID NO: 18. The promoter is upstream of this sequence and can e.g. be retrieved by sequencing or from the cucumber genome database. For example, the at least 1000 or at least 2000 bases upstream of the ATG start include the promoter sequence.

In one aspect the mutant allele of the D14 gene is a mutant allele resulting in reduced expression or no expression of the D14 gene or is a mutant allele resulting in one or more amino acids of the encoded D14 protein being replaced, inserted, duplicated or deleted, compared to the wild type D14 protein.

In one aspect the mutant allele of the D14 gene is obtainable by inducing mutations, either targeted or random, into the gene (promoter or other regulatory elements, splice sites, coding region, etc.) and selecting plants, e.g. from the progeny, comprising a mutant D14 allele. In one aspect an allele comprising a mutation in a codon, or comprising an insertion, deletion or duplication of one or more codons, e.g. of one or more of the codons encoding amino acids 94 to 101 of SEQ ID NO: 2, 8 or 9, is selected. In one aspect the mutant allele causes a truncation of the encoded watermelon, cucumber or melon D14 protein.

In one aspect the INDEL marker (marker mWM23349015_k2) is detected in the genome of a watermelon plant or plant part, or DNA therefrom. This INDEL marker is described in the Examples and detects the insertion allele (comprising 24 nucleotides inserted/duplicated, resulting in the duplication of 8 amino acids) and/or the wild type allele in watermelon.

It is noted that reference to the INDEL marker mWM23349015_k2 is not limited to the specific forward and reverse PCR primers provided herein, but relates to any bi-allelic marker which can differentiate between the wild type ClD14 allele of SEQ ID NO: 6 and the mutant ClD14 allele of SEQ ID NO: 5 (comprising 24 nucleotides duplicated/inserted). The skilled person can easily make other allele-specific primers or allele-specific probes to be used as bi-allelic marker for detecting the genotypes for these two ClD14 alleles.

In one aspect the INDEL marker (marker mWM23349015_k2) is detected in the genome of a watermelon plant or plant part, or genomic DNA or cDNA therefrom. Therefore, a method for detecting the presence of an insertion of 24 nucleotides is provided herein. Thus, genomic DNA or cDNA/mRNA of watermelon can be screened for the presence of the wild type ClD14 allele and/or the insertion allele and can optionally be selected.

In another aspect the SNP that confers the single amino acid replacement by another amino acid or by a stop codon as shown in Table A or Table 2 is detected in the genome of a watermelon plant or plant part, or DNA therefrom. Therefore, a method for detecting the presence of any of those SNPs is provided herein. Thus, genomic DNA or cDNA/mRNA of watermelon can be screened for the presence of the wild type ClD14 allele and/or the mutant allele of Table A or Table 2 and can optionally be selected.

For other mutant D14 alleles of watermelon, cucumber or melon, INDEL or SNP markers (or other markers) and INDEL or SNP genotyping (or other genotyping) assays can easily be designed. Thus, allele specific markers and detection methods are encompassed herein, especially for any mutant allele which results in an amino acid insertion, duplication, deletion or replacement of a D14 protein of watermelon, cucumber or melon.

Especially in one aspect the genotype of the INDEL marker (e.g. marker mWM23349015_k2) can be determined and used to select plants or progeny plants comprising the wild type allele of SEQ ID NO: 6 and/or the mutant C/D114 allele of SEQ ID NO: 5.

The diploid plant heterozygous for the mutant ClD14 allele will comprise both SEQ ID NO 5 and SEQ ID NO: 6 in the genome. The diploid plant homozygous for the mutant ClD14 allele will comprise only SEQ ID NO: 5 at the locus on chromosome 8. And the diploid plant homozygous for the wild type allele will comprise only SEQ ID NO: 6 in the genome.

As mentioned, mutant-allele-specific markers and marker assays can equally easily be developed for any mutant D14 allele, as the underlying genomic change, e.g. in a codon, can be used to design a marker assay to detect the genomic change, e.g. underlying the amino acid changes disclosed herein or other genomic changes in the mutant D14 allele compared to the wild type D14 allele.

Using such allele-specific markers, which detect specific mutant D14 alleles, genotyping can be carried out to detect the presence and copy number of the allele in plants and plant material (or DNA derived therefrom).

Concerning the embodiments of the invention, the mutation in the mutant allele of a D14 gene can be any mutation, including deletions, truncations, insertions, point mutations, nonsense mutations, missense or non-synonymous mutations, splice-site mutations, frame shift mutations and/or mutations in regulatory sequences. In one aspect the mutation in the mutant allele of a D14 gene is a point mutation. The mutation can occur in a DNA sequence comprising the coding sequence of a D14 gene or in an RNA sequence encoding a D14 protein or it can occur in the amino acid of a D14 protein. Concerning a DNA sequence of a D14 protein-encoding gene the mutation can occur in the coding sequence or it can occur in non-coding sequences like 5′- and 3′-untranslated regions, promoters, enhancers etc. of a D14 gene. In respect to RNA encoding a D14 protein the mutation can occur in the pre-mRNA or the mRNA. In one aspect the mutant allele results in the protein having a loss-of-function or decrease of function due to one or more amino acids being replaced, inserted, duplicated and/or deleted, for example resulting in one or more amino acids being replaced, inserted, duplicated and/or deleted at the C-terminal end of the protein, in the IPR000073 domain, in the helical lid domain or comprising one of the amino acids of the catalytic triad.

One embodiment of the invention, therefore, concerns plant cells or plants according to the invention comprising a mutant allele of a D14 gene characterized in that the mutant allele comprises or effects one or more of the mutations selected from the group consisting of
a) a deletion, truncation, insertion, point mutation, nonsense mutation, missense or non-synonymous mutation, splice-site mutation, frame shift mutation in the genomic sequence;
b) a mutation in one or more regulatory sequences;
c) a deletion, truncation, insertion, point mutation, nonsense mutation, missense or non-synonymous mutation, splice-site mutation, frame shift mutation in the coding sequence;
d) a deletion, truncation, insertion, point mutation, nonsense mutation, missense or non-synonymous mutation, splice-site mutation, frame shift mutation in the pre-mRNA or mRNA; and/or
e) a deletion, truncation, insertion, duplication or replacement of one or more amino acids in the D14 protein.

In one aspect the mutant allele results in reduced expression or no expression of the D14 gene or the mutant allele encodes a protein having a decreased function or a loss-of-function. In particular, the homozygous form of the mutant allele results in a significant increase in the average number of secondary branches in a plant homozygous for the mutant allele, in comparison to a control plant homozygous for the wild type allele. The significant increase in average secondary branching is either ‘full multibranching’ when the allele is a knock-out allele or produces a non-functional protein, or ‘intermediate multibranching when the allele is a knock-down allele or produces a reduced-function protein.

Reduced expression (knock-down allele) or no expression (knock-out allele) means that there is a mutation in a regulatory region of the D14 gene, such as the promoter, whereby reduced mRNA transcript or no mRNA transcript of the D14 allele is being made, compared to plants and plant parts comprising a wild type D14 allele. The decrease in the expression can, for example, be determined by measuring the quantity of mRNA transcripts encoding D14 protein, e.g. using Northern blot analysis or RT-PCR. Here, a reduction preferably means a reduction in the amount of RNA transcripts by at least 50%, in particular by at least 70%, optionally by at least 85% or by at least 95%, or even by 100% (no expression) compared to the plant or plant part comprising a wild type D14 gene. Expression can be analyzed e.g. in flower tissue or leaf tissue.

In one aspect the protein comprising one or more amino acids replaced, inserted, duplicated or deleted compared to the wild type protein. Thus, for watermelon, cucumber or melon, one or more amino acids are inserted, deleted or replaced compared to the wild type D14 protein of SEQ ID NO: 2, 8 or 9 or a wild type D14 protein comprising at least 95%, 96%, 97%, or 98% or 99% sequence identity to SEQ ID NO: 2, 8 or 9, whereby the mutant protein has reduced function or loss of function compared to the wild type protein and thus results in (intermediate or strong) multibranching when the mutant allele is present in homozygous form in a diploid plant. The mutant alleles of the above wild type alleles are in one aspect mutant alleles having reduced expression or no expression (through e.g. mutations in the promoter or enhancer elements) or producing a mutant protein which comprises one or more amino acids inserted, duplicated, deleted or replaced compared to the wild type protein, whereby the mutant protein has a reduced function or no function in vivo, as can be determined when the mutant allele is in homozygous form in a plant and by analysing the phenotype of the plant homozygous for the mutant allele compared to the plant homozygous for the wild type allele. The same phenotypic analysis can be done for a mutant allele having reduced gene expression or no gene expression. Thus, any mutant allele can be made homozygous in the plant and the phenotype can be compared to the control plant comprising the original, non-mutated allele and/or to a control plant comprising a mutant allele encoding a non-functional protein (such as e.g. ClD14ins or W155*, or the same mutants in cucumber or melon D14 proteins).

When amino acids from one amino acid to another amino acid are mentioned herein this includes the start/first and end/last amino acid mentioned.

When referring to an amino acid being ‘deleted’, this includes a mutation whereby the codon is changed into a stop codon, or the codon is deleted, or a mutation whereby there is a frameshift, resulting in the amino acid not being encoded. Equally, when referring to an amino acid being ‘replaced’, this includes a mutation whereby the codon encodes a different amino acid, or a codon is inserted, or a mutation whereby there is a frameshift resulting in a different amino acid being encoded.

Watermelon may be any type of watermelon. In one aspect the watermelon plant comprising one or two copies of a mutant ClD14 allele, e.g. the mutant allele encoding a protein of SEQ ID NO: 1, or a different mutant allele, is not a pollenizer plant, i.e. it is not suitable as a pollenizer for triploid fruit production, e.g. because flowering time is not synchronized with the triploid flowering and/or pollen production is not sufficiently high to be suitable as pollen producer. In one aspect it is used for fruit production itself and not for pollen production. Thus, it is not interplanted (and not suitable for interplanting) with triploid plants but grown by itself for fruit production via self pollination. The fruits produced after self-pollination are also not ‘non-harvestable’ with pink or white fruit flesh and low brix but are well suited for harvesting and consumption (i.e. have a high brix, red fruit flesh, etc.).

Watermelon plants, and parts thereof, which comprises at least one copy of the mutant D14 allele, may be diploid, tetraploid or triploid. A diploid plant may be heterozygous for the mutant allele or homozygous for the mutant allele, e.g. for the mutant allele encoding the protein of SEQ ID NO: 1 or any other mutant allele described. In one aspect the diploid plant comprising the mutant D14 allele in homozygous form is a double haploid plant (DH), e.g. a double haploid watermelon, cucumber or melon plant or plant cell or plant part.

A triploid watermelon plant may have one, two or three copies of the mutant ClD14 allele. The triploid plant with one copy of a mutant allele can be made by crossing a wild type female tetraploid (with 4 wild type copies) with a diploid male homozygous for the mutant allele. The triploid plant with two mutant alleles can be made by crossing a female tetraploid comprising four copies of a mutant allele with a diploid male homozygous for the wild type allele.

A tetraploid watermelon plant may have one, two, three or four copies of the mutant ClD14 allele. The genotypes comprising two copies of the mutant allele can be made by doubling the chromosomes of a diploid which is heterozygous for the mutant allele. The genotypes comprising four copies of the mutant allele can be made by doubling the chromosomes of a diploid which is homozygous for the mutant allele.

The watermelon, cucumber or melon plants encompassed herein can also be reproduced vegetatively (clonally) and such vegetatively propagated plants, or ‘vegetative propagations’ are an embodiment of the invention. They can easily be distinguished from other plants by the presence of a mutant D14 allele and/or phenotypically (optionally after selfing). The presence of one or more mutant D14 alleles can be determined as described elsewhere herein.

Vegetative propagations can be made by different methods. For example one or more scions of a plant of the invention may be grafted onto a different rootstock, e.g. a biotic or abiotic stress tolerant rootstock.

Other methods include in vitro cell or tissue culture methods and regeneration of vegetative propagations from such cultures. Such cell or tissue cultures comprise or consist of various cells or tissues of a plant of the invention. In one aspect such a cell or tissue culture comprises or consists of vegetative cells or vegetative tissues of a plant of the invention.

In another aspect a cell or tissue culture comprises or consists of reproductive cells or tissues, such as anthers, pollen, microspores or ovules of a plant of the invention. Such cultures can be treated with chromosome doubling agents to make e.g. double haploid plants, or they can alternatively be used to make haploid plants (e.g. to make diploids from a tetraploid or to make haploids from a diploid).

An in vitro cell or tissue culture may, thus, comprise or consist of cells or protoplasts or plant tissue from a plant part selected from the group consisting of: fruit, embryo, meristem, cotyledon, pollen, microspores, ovule, leaf, anther, root, root tip, pistil, flower, seed, stem. Also parts of any of these are included, such as e.g. only the seed coat (maternal tissue).

Thus, in one aspect of the invention a cell culture or a tissue culture of cells of a plant comprising one or two copies of a mutant D14 allele, all as described above, is provided. As mentioned, a cell culture or a tissue culture comprises cells or protoplasts or plant tissue from a plant part of a plant comprising a mutant D14 allele may comprise or consist of cells or tissues selected from the group consisting of: embryo, meristem, cotyledon, pollen, microspore, leaf, anther, root, root tip, pistil, flower, seed, stem: or parts of any of these.

Also provided is a watermelon, cucumber or melon plant regenerated from such a cell culture or tissue culture, wherein the regenerated plant (or progeny thereof, e g, obtained after crossing or selfing the regenerated plant) comprises the mutant D14 allele. Therefore, in one aspect the watermelon, cucumber or melon plant comprising a mutant D14 allele in one or more copies is a vegetatively propagated plant.

In a different aspect the cells and tissues of the invention (and optionally also the cell or tissue culture), comprising a mutant D14 allele in one or more copies, are non-propagating cells or tissues.

### FURTHER METHODS

Also provided is a method for production of a watermelon, cucumber or melon plant capable of producing an increased average number of secondary branches, or a method for producing mutant alleles of the D14 gene, comprising the steps of
a) introducing mutations in a population of watermelon, cucumber or melon plants or providing a mutant population of watermelon, cucumber or melon plants; or providing watermelon, cucumber or melon plants comprising randomly induced mutations or targeted induced mutations in the D14 target gene,
b) selecting a plant comprising a mutant allele of the D14 gene;
c) optionally verifying if the plant selected under b) produces an increased average number of secondary branches when the mutant D14 allele is in homozygous form, compared to a control plant which comprises the wild type alleles of the D14 gene.

Also provided is a method for production of a watermelon, cucumber or melon plant capable of producing a full multibranching phenotype, or a method for producing mutant alleles of the D14 gene which confer a full multibranching phenotype when in homozygous form, comprising the steps of
a) introducing mutations in a population of watermelon, cucumber or melon plants or providing a mutant population of watermelon, cucumber or melon plants; or providing watermelon, cucumber or melon plants comprising randomly induced mutations or targeted induced mutations in the D14 target gene,
b) selecting a plant comprising a mutant allele of the D14 gene, whereby the mutant D14 allele is a knock-out allele or an allele which encodes a loss-of-function D14 protein;
c) optionally verifying if the plant selected under b) produces an increased average number of secondary branches when the mutant D14 allele is in homozygous form, compared to a control plant which comprises the wild type alleles of the D14 gene.

Further provided is a method for production of a watermelon, cucumber or melon plant capable of producing an intermediate multibranching phenotype, or a method for producing mutant alleles of the D14 gene which confer an intermediate multibranching phenotype when in homozygous form, comprising the steps of
a) introducing mutations in a population of watermelon, cucumber or melon plants or providing a mutant population of watermelon, cucumber or melon plants; or providing watermelon, cucumber or melon plants comprising randomly induced mutations or targeted induced mutations in the D14 target gene,
b) selecting a plant comprising a mutant allele of the D14 gene, whereby the mutant D14 allele is a knock-down allele or an allele which encodes a reduced-function D14 protein;
c) optionally verifying if the plant selected under b) produces an increased average number of secondary branches when the mutant D14 allele is in homozygous form, compared to a control plant which comprises the wild type alleles of the D14 gene.

A watermelon, melon or cucumber plant comprising at least one copy of a mutant D14 allele produced by the above method and/or a mutant D14 allele induced and identified by the above method is encompassed. In one aspect the watermelon plant produced by the above method, and comprising a mutant allele which confers full multibranching in homozygous form, does not comprise the mutant allele of SEQ ID NO: 5. In another aspect the watermelon plant produced by the above method, and comprising a mutant allele which confers full multibranching in homozygous form, is in a different watermelon background than variety Sidekick and differs in one or more characteristics from variety Sidekick, in case it does encode the same protein as encoded by the pollenizer Sidekick (protein ClD14ins shown in SEQ ID NO: 1). It may for example differ from Sidekick in not being suitable as a pollenizer, and/or in producing fruits having red fruit flesh, and/or having higher average fruit weight, or other characteristics which distinguish the plant from Sidekick.

The population of watermelon, cucumber or melon plants under a) is preferably a single genotype of a cultivated watermelon, cucumber or melon breeding line or variety, which is treated/has been treated with (or subjected to) a mutagenic agent, or progeny of such a population e.g. obtained after selfing individuals of the population to produce M2, M3 or further generation plants. This may for example be a TILLING population. It may also be a watermelon, cucumber or melon line which has been subjected to targeted gene modification using e.g. Crispr based methods.

In step b) the selection of a plant comprising a mutant allele of the D14 gene can be carried out phenotypically and/or by screening the plants (or plant parts or DNA therefrom) for the presence of a mutant allele of the D14 gene, i.e. an allele which either has reduced expression (in case of a knock-down allele) or no expression (in case of a knock-out allele) of the wild type D14 allele or an allele encoding a mutant D14 protein.

Regarding the screening for the phenotype, it is understood that these can only be selected if the mutant D14 allele is in homozygous form and if the mutant allele has reduced expression or no expression or encodes a reduced function or loss-of-function protein, so that the phenotype is seen. The screening for the phenotype or combination of phenotypes can be done as described, e.g. growing a line comprising the mutant D14 allele in homozygous form under the same growth conditions as a control line or variety comprising the wild type D14 allele in homozygous form and then analysing secondary branching.

Regarding the screening or selection of the plants for the presence of a mutant allele of the D14 gene, this can be done by various methods which detect D14 DNA, RNA or protein, for example by e.g. designing PCR primers which amplify part of the coding region or all of the coding region to amplify the genomic DNA in order to determine if a plant comprises a mutation in the genomic DNA, or other methods.

Thus, to determine the presence or select a plant comprising a mutant D14 allele is present various methods can be used. For example marker analysis or sequence analysis of the allele or of the chromosome region comprising the D14 locus can be carried out, or PCR or RT-PCR can be used to amplify the D14 allele (or a part thereof) or the mRNA (cDNA) or sequencing can be done. Also, genetic analysis to determine the recessive inheritance may be carried out. The allele can, thus, for example be sequenced (e.g. its genomic DNA or cDNA) to determine what mutation is present. In step b) also Provean and/or SIFT analysis can be used to select a plant having a mutant allele which is predicted to reduce or abolish D14 protein function. See Examples.

If gene editing methods have been used, the vector/construct that has been introduced into the plant to induce the mutations in the endogenous allele is preferably removed from the plant line comprising the mutant D14 allele, so that the plant line does not comprise such a vector or construct.

In one aspect the plants do not contain a genetic construct inserted into the genome through transformation.

In one aspect the mutant alleles are generated by mutagenesis (e.g. chemical or radiation mutagenesis) or by targeted mutagenesis, especially using the CRISPR system (e.g. Crispr/Cas9 or Crispr/Cpf1 or other nucleases). In one aspect the cultivated plant comprising the mutant D14 allele is not a transgenic plant, i.e. non transgenic progeny are selected which do not comprise e.g. the CRISPR construct.

In one aspect the mutant allele of the D14 gene comprises a human induced mutation, i.e. a mutation introduced by mutagenesis techniques, such as chemical mutagenesis or radiation mutagenesis, or targeted mutagenesis techniques, such as Crispr based techniques.

A method for targeted mutagenesis of the endogenous D14 gene in watermelon, cucumber or melon is provided herein, using any targeted gene modification method, such as CRISPR based methods (e.g. Crispr/Cas9 or Crispr/Cpf1), TALENS, Zinc Fingers or other methods.

In one aspect an isolated mutant D14 protein and an isolated wild type D14 protein is provided or an isolated nucleic acid molecule encoding a mutant D14 protein or a wild type D14 protein. Also an antibody able to bind a mutant or wild type D14 protein is encompassed herein. The isolated mutant protein is in one aspect the protein of SEQ ID NO: 1, comprising a duplication of 8 amino acids, but may also be an isolated protein of any other mutant D14 allele described herein. In one aspect the isolated mutant protein is a protein described in Table A or Table 2. In one aspect the isolated nucleic acid is a DNA or RNA encoding a mutant protein described in Table A or Table 2.

In a further aspect fragments of the nucleotide sequences, or nucleic acid molecules, provided herein (and/or of the complement strand of the sequences or molecules) are encompassed, as these can be used as PCR primers or probes to detect the sequences in DNA or RNA samples. Fragments include for example stretches of at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65 or more nucleotides of the genomic sequences of SEQ ID NO: 5 or 6, SEQ ID NO: 13 or 14, SEQ ID NO: 15 or 16, SEQ ID NO: 10, 11 or 12, or the complement strand or reverse complement strand of any of these, or of the mRNA or cDNA sequences, or molecules, of SEQ ID NO: 3 or 4, or SEQ ID NO: 17 or 18, or the complement strand or reverse complement strand of any of these. Also fragments of the isolated nucleic acid molecules or sequences (DNA or RNA) encoding a mutant protein described in Table A or Table 2 are encompassed.

### Detection Methods

In one aspect a screening method for identifying and/or selecting seeds, plants or plant parts or DNA from such seeds, plants or plant parts comprising in their genome a mutant allele and/or a wild type allele of a D14 protein-encoding gene is provided.

The method comprises screening at the DNA (especially genomic DNA), RNA (or cDNA) or protein level using known methods, in order to detect the presence of the mutant allele and/or of the wild type allele. There are many methods to detect the presence of a mutant and/or wild type allele of a gene.

Thus, a method for screening and/or selecting plants or plant material or plant parts, or DNA or RNA or protein derived therefrom, for the presence of a mutant D14 allele and/or a wild type D14 allele is provided comprising one or more of the following steps:
a) determining the gene expression of the endogenous D14 gene, e.g. to detect if it is reduced or abolished;
b) determining the amount of wild type D14 protein, e.g. to detect if it is reduced or abolished;
c) determining if a mutant and/or wild type mRNA, cDNA or genomic DNA encoding a mutant or wild type D14 protein is present;
d) determining if a mutant and/or wild type D14 protein is present;
e) determining if plants or progeny thereof show a mutant phenotype (as described. e.g. strong multibranching or intermediate multibranching) or a wild type phenotype (normal branching).

Routine methods can be used, such as RT-PCR, PCR, antibody-based assays, sequencing, genotyping assays (e g allele-specific genotyping), genotyping-by-sequencing, phenotyping, etc.

The plants or plant material or plant parts may be watermelon, cucumber or melon plants or plant materials or plant parts, such as leaves, leaf parts, cells, fruits, fruit parts, ovaries, stem, hypocotyl, seed, parts of seeds, seed coat, embryo, etc.

For example, if there is a single nucleotide difference (single nucleotide polymorphism, SNP, or Insertion Deletion polymorphism. InDel) between the wild type allele and the mutant allele, a SNP or InDel genotyping assay can be used to detect whether a plant or plant part or cell comprises the wild type nucleotide (or nucleotides) or the mutant nucleotide (or nucleotides) in its genome. For example, the SNP or INDEL can easily be detected using a KASP-assay (see world wide web at kpbioscience.co.uk) or other genotyping assays, especially bi-allelic genotyping assays. For developing a KASP-assay, for example about 70 base pairs upstream and about 70 base pairs downstream of the SNP or INDEL can be selected and two allele-specific forward primers and one reverse primer can be designed. See e.g. Allen et al. 2011, Plant Biotechnology J. 9, 1086-1099, especially p 097-1098 for KASP-assay method.

Equally other genotyping assays can be used. For example, a TaqMan SNP genotyping assay, a High Resolution Melting (HRM) assay, SNP-genotyping arrays e.g. microarrays (e.g. Fluidigm, Illumina, etc.) or DNA sequencing (e.g. genotyping by sequencing) may equally be used.

Thus, based on the difference between the genomic sequence of the wild type allele and the mutant allele, the skilled person can easily develop markers or assays which can be used to detect specific alleles.

Also provided herein is a method for identifying a watermelon, cucumber or melon plant (or plant part) comprising a mutant D14 allele, the method comprising detecting in the plant (or plant part) the presence of a mutant D14 allele, wherein the presence is detected by at least one marker (e.g. SNP marker or InDel marker) within the D14 allele or by detecting the protein encoded by the D14 allele. The method for detecting the mutant D14 allele is selected from the group consisting of methods comprising PCR amplification, nucleic acid sequencing, nucleic acid hybridization and an antibody-based assay (e.g. immunoassay) for detecting the D14 protein encoded by the allele.

Also provided herein is a method for identifying a watermelon, cucumber or melon plant (or plant part) comprising a mutant D14 allele comprising a mutation in a regulatory element, the method comprising detecting in the plant (or plant part) the reduced gene expression or absence of gene expression of the mutant D14 allele, wherein the presence is detected by mRNA levels (cDNA) of the wild type D14 allele or by detecting the protein levels of the wild type D14 protein. The method for detecting the mutant D14 allele is selected from the group consisting of PCR amplification (e.g. RT-PCR), nucleic acid sequencing, western blotting and an antibody based assay (e.g. immunoassay) for detecting the D14 protein encoded by the allele.

Also provided is a method for determining, or detecting or assaying, whether a cell or of a watermelon, cucumber or melon plant or plant part comprises a mutant allele of a gene name D14 encoding a protein of SEQ ID NO: 2, 8 or 9, or a protein comprising at least 95%, 9%%, 97%, 98%, 99% or 100/% sequence identity to SEQ ID NO: 2, 8 or 9, is provided herein. In one aspect the method comprises determining the expression of the allele, and/or determining the coding sequence of the allele and/or determining part of the coding sequence of the allele (e.g. a SNP or INDEL genotype of the allele), and/or determining the amino acid sequence of the protein produced and/or the amount of protein produced.

Various method can be used to determine whether a plant or part thereof comprises a mutant D14 allele of the invention. As mentioned, the mRNA (or cDNA) level of the wild type allele may be determined, or the wild type protein level may be determined, to see if there is a reduced expression or no expression of the wild type allele. Also, the coding sequence or part thereof may be analyzed, for example if one already knows which mutant allele may be present, an assay can be developed to detect the mutation, e.g. a SNP or INDEL genotyping assay can e.g. distinguish between the presence of the mutant allele and the wild type allele, e.g. genotyping for marker mWM23349015_k2 (see Examples) or genotyping for any of the mutant alleles of Table A or Table 2, or other mutant alleles.

A method for selection of a plant comprising the steps of:
a) identifying a plant which has a mutation in an allele encoding a D14 protein-encoding gene, wherein the wild type allele of the gene encodes a D14 protein comprising at least 95%, 96%, 97%, 98%, 99% or 100% sequence identity to any one of the proteins selected from the group of: SEQ ID NO: 2, 8 or 9, and optionally
b) determining whether the plant, or a progeny plant, produces a multibranching phenotype and optionally
c) selecting a plant comprising at least on copy of the mutant allele of step a).

A method for production of a plant comprising the steps of:
a) introducing mutations in a population of plants or providing a population of mutated plants (e g, a TILLING population),
b) selecting a plant producing a multibranching phenotype and/or comprising a mutant D14 allele,
c) optionally verifying if the plant selected under b) has a mutation in an allele encoding a D14 protein-encoding gene and selecting a plant comprising such a mutation, and optionally
d) growing/cultivating the plants obtained under c), wherein the wild type allele of the gene encodes a D14 protein comprising at least 95% sequence identity to the protein of: SEQ ID NO:2, 8 or 9.

A method for selection of a plant comprising a strong multibranching phenotype or an intermediate multibranching phenotype comprising the steps of:
a) screening plants (or DNA therefrom) for the presence of mutant alleles of the D14 gene, wherein the wild type allele of the gene encodes a D14 protein comprising at least 95%, 96%, 97%, 98%, 99% or 100% sequence identity to any one of the proteins selected from the group of: SEQ ID NO: 2, 8 or 9, and
b) selecting a plant comprising a mutant allele which either i) is a knock-out allele or encodes a non-functional D14 protein, which allele in homozygous form produces a strong multibranching phenotype or which ii) is a knock-down allele or encodes a reduced-function D14 protein, which allele in homozygous form produces an intermediate multibranching phenotype, and optionally
c) confirming that the plant, or a progeny plant comprising the mutant allele in homozygous form, produces said strong multibranching phenotype of i) or said intermediate multibranching phenotype of ii.

In one aspect step b) comprises predicting whether the mutant allele encodes a D14 protein that has reduced function or a loss-of function by e.g. carrying out SIFT or Provean analysis of the effect of the amino acid change(es) on protein function. The plant or plants comprising an allele that is predicted to be ‘deleterious’ in Provean analysis and/or ‘not tolerated’ in SIFT analysis are selected in step b).

A method for production or selection of a plant comprising a strong multibranching phenotype or an intermediate multibranching phenotype comprising the steps of:
a) introducing mutations in a population of plants, or providing a population of mutant plants (e.g. a TILLING population),
b) selecting a plant comprising a mutant D14 allele, which either i) is a knock-out allele or encodes a non-functional D14 protein, which allele in homozygous form produces a strong multibranching phenotype or which ii) is a knock-down allele or encodes a reduced-function D14 protein, which allele in homozygous form produces an intermediate multibranching phenotype,
c) selecting a plant comprising a mutant allele of i) or ii),

wherein the wild type allele of the gene encodes a D14 protein comprising at least 95% sequence identity to the protein of: SEQ ID NO: 2, 8 or 9.

The selected plant may be selfed to produce a plant which comprises the mutant allele in homozygous form, and the homozygous plant may be grown to determine the phenotype.

A method for production of a plant comprising the steps of:
a) introduction of a foreign nucleic acid molecule into a plant, wherein the foreign nucleic acid molecule is chosen from the group consisting of
i) DNA molecules, which code at least one antisense RNA, which effects a reduction in the expression of an endogenous gene encoding a D14 protein;
ii) DNA molecules, which by means of a co-suppression effect lead to the reduction in the expression of an endogenous gene encoding a D14 protein;
iii) DNA molecules, which code at least one ribozyme, which splits specific transcripts of an endogenous gene encoding a D14 protein;
iv) DNA molecules, which simultaneously code at least one antisense RNA and at least one sense RNA, wherein the said antisense RNA and the said sense RNA form a double-stranded RNA molecule, which effects a reduction in the expression of an endogenous gene encoding a D14 protein (RNAi technology);
v) nucleic acid molecules introduced by means of in vivo mutagenesis, which lead to a mutation, or an insertion of a heterologous sequence, in an endogenous gene encoding a D14 protein, wherein the mutation or insertion effects a reduction in the expression of a gene encoding a D14 protein or results in the synthesis of a loss-of-function or reduced function D14 protein;
vi) nucleic acid molecules, which code an antibody, wherein the antibody results in a reduction in the activity of an endogenous gene encoding a D14 protein due to the bonding of the antibody to an endogenous D14 protein;
vii) DNA molecules, which contain transposons, wherein the integration of these transposons leads to a mutation or an insertion in an endogenous gene encoding a D14 protein, which effects a reduction in the expression of an endogenous gene encoding a D14 protein, or results in the synthesis of an inactive protein;
viii) T-DNA molecules, which, due to insertion in an endogenous gene encoding a D14 protein, effect a reduction in the expression of an endogenous gene encoding a D14 protein, or result in the synthesis of a loss-of-function or reduced function D14 protein;
ix) nucleic acid molecules encoding rare-cleaving endonucleases or custom-tailored rare-cleaving endonucleases preferably a meganuclease, a TALENs or a CRISPR/Cas system.
b) selecting a plant or progeny of a plant, wherein the plant, or a progeny of the plant, produces a higher percentage of male flowers and/or flowers with fused petals and/or leaves, optionally
c) verifying if the plant or progeny selected under b) has a decreased activity of a D14 protein compared to wild type plants into whose genome e.g. no foreign nucleic acid molecules had been integrated, optionally
d) growing/cultivating the plants obtained under c).

A plant obtained by any of the methods above is encompassed herein.

In one aspect a genetically modified plant and plant part is provided, whereby the plant has reduced expression or no expression of the endogenous D14 gene, e.g. through silencing of the endogenous D114 gene. Such a plant may be any plant, in one aspect it is a watermelon, cucumber or melon.

In another aspect a watermelon, cucumber or melon plant and plant part is provided, comprising a mutation in the endogenous D14 gene, e.g. an induced mutation generated e.g. by targeted mutagenesis, whereby either the gene expression is reduced or abolished, or the expressed gene encodes a reduced function or loss of function D14 protein compared to the wild type protein.

In another aspect a method is provided for detecting, and optionally selecting, a watermelon plant, seed or plant part comprising at least one copy of a wild type allele and/or of a mutant allele of a gene name (ClD14 (Citrullus lanatus Dwarf14), comprising the steps of:
a) providing one or more genomic DNA samples of one or more watermelon plants, seeds or plant parts,
b) carrying out a genotyping assay, using the DNA samples of a) as template, that discriminates between the wild type ClD14 allele and the mutant ClD14 allele, wherein said genotyping assay is based on nucleic acid amplification making use of ClD14 allele-specific oligonucleotide primers, and/or wherein said genotyping assay is based on nucleic acid hybridization making use of ClD14 allele-specific oligonucleotide probes, and optionally
c) selecting a plant, seed or plant part comprising one or two copies of the mutant allele, wherein the wild type ClD14 allele comprises the sequence of SEQ ID NO: 6 and the mutant ClD14 allele comprises one or more nucleotides inserted, duplicated, deleted or replaced with respect to the sequence of SEQ ID NO: 6.

In the method above, ClD14 allele-specific oligonucleotide primers or said ClD14 allele-specific oligonucleotide probes may be used which comprise at least 10, 11, 12, 13, 14, 15 or more nucleotides of SEQ ID NO: 6 or of the complement strand of SEQ ID NO: 6.

In one aspect in the method above the mutant allele comprises at least one codon inserted or duplicated in the coding region of the allele, or at least one codon changed into another codon, or at least one codon deleted or changed into a STOP codon.

In one aspect of the above method the mutant allele comprises the sequence of SEQ ID NO 5.

In another aspect of the above method the mutant allele is an allele encoding a protein as described in Table A or Table 2.

In another aspect of the above method the mutant allele is an allele encoding a loss-of-function D14 protein or a reduced-function D14 protein, as described elsewhere herein.

In one aspect in the above method the oligonucleotide primers or oligonucleotide probes comprise at least 15, 16, 17 or more nucleotides complementary to SEQ ID NO: 6 or to the complementary sequence of SEQ ID NO: 6.

Preferably the genotyping assay used in the above method is a KASP-assay, said KASP-assay comprises a first forward primer detecting the wild type allele of SEQ ID NO: 6 in the DNA sample, a second forward primer detecting the mutant allele comprising one or more nucleotides inserted, deleted or replaced with respect to SEQ ID NO: 6 in the DNA sample, and one common reverse primer.

In one aspect the second forward primer detects the mutant allele of SEQ ID NO: 5 in the DNA sample.

In another aspect the second forward primer detects the mutant allele, which is an allele encoding a protein as described in Table A or Table 2.

In another aspect the second forward primer detects the mutant allele, which is an allele encoding a loss-of-function D14 protein or a reduced-function D14 protein, as described elsewhere herein.

In one aspect of the KASP assay the first forward primer comprises SEQ ID NO: 11 or the complementary sequence thereof and/or the second forward primer comprises SEQ ID NO: 10 or the complementary sequence thereof.

Also encompassed herein is a synthesized or synthetic nucleic acid primer or probe, wherein said primer or probe comprises at least 15 nucleotides of e.g. SEQ ID NO: 5 (or another mutant allele) or of SEQ ID NO: 6, or of the complement sequence of either of these. Such oligos can be synthesized using common methods for oligo synthesis. The primers or probes are preferably DNA oligos and provided e.g. in a buffer solution, to be used in e.g. a genotyping assay.

Further provided is a method for detecting, and optionally selecting, a watermelon, cucumber or melon plant, seed or plant part comprising at least one copy of a wild type D14 allele and/or of a mutant D14 allele of a gene name ClD14 (Citrullus lanatus Dwarf14), CsD14 (Cucumis sativus Dwarf14) or CmD14 (Cucumis melo Dwarf14) comprising the steps of:
a) providing one or more genomic DNA samples of one or more watermelon, cucumber or melon plants, seeds or plant parts,
b) carrying out a genotyping assay, using the DNA samples of a) as template, wherein said genotyping assay is based on nucleic acid amplification making use of D14 allele-specific oligonucleotide primers, and/or wherein said genotyping assay is based on nucleic acid hybridization making use of D14 allele-specific oligonucleotide probes, and optionally
c) selecting a plant, seed or plant part comprising one or two copies of the mutant allele,

wherein the wild type D14 allele encodes the protein of SEQ ID NO: 2 (in watermelon), SEQ ID NO: 8 (in cucumber) and SEQ ID NO: 9 (in melon) and the mutant D14 allele comprises one or more amino acids inserted, deleted or replaced with respect to SEQ ID NO: 2, SEQ ID NO: 8 or SEQ ID NO: 9.

In one aspect the mutant D14 allele is an allele encoding a loss-of-function D14 protein or a reduced-function D14 protein, as described elsewhere herein.

In one aspect of the method said D14 allele-specific oligonucleotide primers or said D14 allele-specific oligonucleotide probes comprise at least 10, 11, 12, 13, 14, 15, 16, 17, 18, 19, 20 or more nucleotides of SEQ ID NO: 6, or of SEQ ID NO: 15 or of SEQ ID NO: 16, or of the complement strand of any of these.

In a further aspect of the above method the mutant allele encodes a protein comprising a duplication of at least one amino acid selected from amino acids 94 to 101 of SEQ ID NO: 2, SEQ ID NO: 8 and SEQ ID NO: 9. In one aspect the mutant allele encodes a protein comprising a duplication of at least Serine 97 of SEQ ID NO: 2, SEQ ID NO: 8 and SEQ ID NO: 9. In another aspect the mutant allele encodes a protein comprising a duplication of amino acids 94 to 101 of SEQ ID NO: 2, SEQ ID NO: 8 or SEQ ID NO: 9.

Further a breeding method for watermelon is provided comprising marker assisted selection (MAS) using an InDel marker to select watermelon lines, wherein said InDel marker detects in the genomic DNA sample or samples the sequence of SEQ ID NO: 6 (deletion allele, or the complement sequence thereof) and/or the sequence of SEQ ID NO: 5 (insertion allele, or the complement sequence thereof).

Also a breeding method for watermelon is provided comprising marker assisted selection (MAS) using an InDel marker or a SNP marker to select watermelon lines, wherein said InDel marker or SNP marker detects in the genomic DNA sample or samples the allele which encodes the wild type protein of SEQ ID NO: 2 and/or an allele which encodes a mutant protein, comprising one or more amino acids deleted, inserted, duplicated or replaced with respect to the wild type protein of SEQ ID NO: 6. In one aspect the mutant protein is a loss-of-function D14 protein or a reduced-function D14 protein, as described elsewhere herein. In one aspect the mutant protein is the protein of Table A or Table 2.

In one aspect the mutant protein comprises a duplication of one or more amino acids of SEQ ID NO: 94 to 101 of SEQ ID NO: 2. In one aspect the mutant protein comprises the sequence of SEQ ID NO: 1.

In one aspect the InDel marker is marker mWM23349015_k2.

Also a breeding method for cucumber or melon is provided comprising marker assisted selection (MAS) using an InDel marker or a SNP marker to select cucumber or melon lines, wherein said InDel marker or SNP marker detects in the genomic DNA sample or samples the allele which encodes the wild type protein of SEQ ID NO: 8 or 9 and/or an allele which encodes a mutant protein, comprising one or more amino acids deleted, inserted, duplicated or replaced with respect to the wild type protein of SEQ ID NO: 8 or 9. In one aspect the mutant protein comprises a duplication of one or more amino acids of SEQ ID NO: 94 to 101 of SEQ ID NO: 8 or 9.

These methods above can be used to select or detect or breed with any of the mutant D14 alleles described elsewhere herein.

In a different aspect a method for producing a plant, especially a watermelon plant, cucumber plant or melon plant, is provided comprising:
providing a first inbred plant having two copies of a wild type 1) 4 allele.
providing a second inbred plant having two copies of a mutant D14 allele, e.g. the mutant allele of SEQ ID NO: 5 for watermelon, or any other mutant allele described herein.
crossing the first plant with the second plant to produce seeds of an F1 hybrid,
optionally collecting the F1 hybrid seeds.

In a different aspect a method for producing a plant, especially a watermelon plant, cucumber plant or melon plant, is provided comprising:
providing a first inbred plant having two copies of a mutant D14 allele,
providing a second inbred plant having two copies of a mutant D14 allele, e.g. the mutant allele of SEQ ID NO: 5 for watermelon, or any other mutant allele described herein,
crossing the first plant with the second plant to produce seeds of an F1 hybrid,
optionally collecting the F1 hybrid seeds.

In a different aspect a method for producing a plant, especially a watermelon plant, cucumber plant or melon plant, is provided comprising:
providing a first plant having two copies of a wild type D14 allele,
providing a second plant having one or two copies of a mutant D14 allele, e.g. the mutant allele of SEQ ID NO: 5 for watermelon, or any other mutant allele described herein,
crossing the first plant with the second plant to produce seeds of an F1 plant,
selfing the F1 plant to produce an F2 plant or crossing the F1 plant to another plant to produce a progeny plant.
optionally further selfing the F2 plant or further (back-) crossing the F2 plant or the progeny plant of the previous step to produce a further selfing or backcross plant,
optionally selecting a plant having at least one copy of the mutant D14 allele.

In a further embodiment a method for introgressing a mutant D14 allele into a breeding line or variety of watermelon, cucumber or melon, comprising crossing a plant comprising a mutant D14 allele to a plant lacking a mutant D14 allele, backcrossing the F1, F2 or further generation progeny to the recurrent parent and eventually selecting a recurrent parent comprising the mutant D14 allele Optionally MAS may be used to select the mutant and/or wild type D14 allele in the first or second plant, or in any further generation, such as F2, F3 etc. or backcross generation.

These methods above can be used to select or detect or breed with any of the mutant D14 alleles described elsewhere herein.

A seed and/or a plant produced by any of the above methods and comprising at least one, optionally two copies of a mutant D14 allele is encompassed herein.

SEQUENCE DESCRIPTION

| | |
|---|---|
| SEQ ID NO: 1: | mutant D14 protein of watermelon (ClD14ins), comprising an insert |
| SEQ ID NO: 2: | wild type D14 protein of watermelon (ClD14) |
| SEQ ID NO: 3: | cDNA encoding the mutant ClD14ins protein of SEQ ID NO: 1 |
| SEQ ID NO: 4: | cDNA encoding the wild type D14 protein of SEQ ID NO: 2 |
| SEQ ID NO: 5: | genomic DNA encoding the mutant ClD14ins protein of SEQ ID NO: 1, comprising 24 |
| | nucleotides inserted/duplicated |
| SEQ ID NO: 6: | genomic DNA encoding the wild type ClD14 protein, intron from nucleotide 375 to 463 |
| SEQ ID NO: 7: | Arabidopsis thaliana D14 protein |
| SEQ ID NO: 8: | wild type D14 protein of cucumber, Cucumis sativus, CsD14 protein |
| SEQ ID NO: 9: | wild type D14 protein of melon, Cucumis melo, CmD14 protein |
| SEQ ID NO: 10: | FAM primer of KASP assay for marker mWM23349015_k2 |
| SEQ ID NO: 11: | VIC primer of KASP assay for marker mWM23349015_k2 |
| SEQ ID NO: 12: | common reverse primer of KASP assay for marker mWM23349015_k2 |
| SEQ ID NO: 13: | minus strand of ClD14 wild type allele, used to design KASP primers |
| SEQ ID NO: 14: | minus strand of ClD14ins mutant allele (comprising 24 nucleotides inserted/duplicated), |
| | used to design KASP primers |
| SEQ ID NO: 15: | genomic DNA of cucumber wild type CsD14 gene |
| SEQ ID NO: 16: | genomic DNA of melon wild type CmD14 gene |
| SEQ ID NO: 17: | cDNA of cucumber wild type CsD14 gene |
| SEQ ID NO: 18: | cDNA of melon wild type CmD14 gene |

The following non-liminting examples are provided.

### EXAMPLES

### Example 1

QTL mapping for secondary branching (also referred to as ‘multibranching’) was done on an F2 population developed by crossing the multibranching variety Sidekick F1 with a proprietary normal branching watermelon plant.

Phenotyping was done by counting the number of secondary branches starting on the main stem at 90 cm from the crown to the end of the stem. The counting was done for 5 to 7 plants per line/genotype and the average secondary branching was calculated.

It was found that a gene on chromosome 8 caused the multibranching phenotype in Sidekick F1. The gene contained a duplication of 24 nucleotides, which encoded 8 additional amino acids compared to the wild type gene, present in the normal-branching parent. The gene is herein referred to as ClD14. The multibranching phenotype was only seen when the mutant allele of the gene (comprising the 24-nucleotide duplication) was present in homozygous form.

A KASP marker, referred to as mWM23349015_k2, was developed for distinguishing between the wild type allele of the gene, shown in SEQ ID NO: 6, and the mutant allele of the gene comprising 24 additional nucleotides (the insertion is a duplication of 24 nucleotides of the wild type sequence), shown in SEQ ID NO: 5. See FIG. 4 (intron sequence in bold).

F3 populations were analyzed for mWM23349015_k2 and the average number of secondary branches, with the results below:

| | | Average number | Relative increase |
|---|---|---|---|
| | | of secondary | secondary branches |
| | | branches (90 cm | compared to wild type (set |
| | mWM23349015_k2 genotype | from crown) | at 100% branches) |
| F3 | Homozygous for 24 nucleotide | 46.27 | 240% |
| | duplication (ClD14ins/ClD14ins) | | |
| F3 | Heterozygous (ClD14ins/ClD14) | 20.64 | |
| F3 | Homozygous for wild type ClD14 | 19.27 | 100% |
| | allele (ClD14/ClD14) | | |

Thus, the mutant allele of the gene (SEQ ID NO: 5; ClD14ins, comprising an insertion of 24 nucleotides) was responsible for changing the average branching pattern of the watermelon plants comprising the mutant allele in homozygous form to 45 or more secondary branches being formed.

The KASP assay mWM23349015_k2 was carried out using the two forward primers and common/reverse primer:

SEQ ID NO: 10
(Fam primer, 5′GAGACGGAGTGGCCGACC3′)
and
SEQ ID NO: 11
(VIC primer, 5′GGAGACGGAGTGGCCGACA3′)
SEQ ID NO: 12
(Common primer 5′CACGTCCACCGCTGCGCCTT3′).

The Fam and Vic primers also contained a tail sequence at the 5′end as described for KASP-assays.

It is noted that the DNA sequences for the KASP assay were designed on the reverse DNA strand (minus strand) but can be equally designed based on the plus strand of the allele. Plus and minus strands are complementary strands of the double stranded DNA. Nucleotide G is a C in the complementary strand and nucleotide A is a T in the complementary strand.

The DNA sequences used for KASP assay primer design, mWM23349015 k2 (with FAM and VIC primers shaded grey):

| | |
|---|---|
| Wild | 5′GAGTTCGGGACGOCGOATGGAGGCGAGGATGCCGA |
| type | CCATGGCGGAGACGGAGTGGCCGACAAAGGCGCAGCG |
| allele | GTGGACGTGGAGAGAGTCKAGGATGGAGATGAG |
| (minus | ATCGTCRACGAAGGCG 3′ |
| strand) | (SEQ ID NO: 13, sequence template of |
| | wild type allele, comprising |
| | no insertion) |
| | (Italicized is the VIC primer) |
| Insertion | 5′GAGTTCGGGACGGCGGATGGAGGCGAGGATGCCGA |
| allele | CCATGGCGGAGACGGAGTGGCCGACCATGGCGGAGAC |
| (minus | GGAGTGGCCGACAAAGGCGCAGCGGTGGACGTGGAGA |
| strand) | GAGTCKAGGATGGAGATGAGATCGTCRACGAAGGC |
| | G 3′ |
| | (SEQ ID NO: 14, sequence template of |
| | mutant allele, comprising an insertion |
| | of 24 nucleotides, underlined) |
| | (Italicized is the Fam primer) |

According to the KASP brochure, KASP™ genotyping technology from LOC, Biosearch Technologies™ utilizes a unique form of competitive allele-specific PCR (polymerase chain reaction) that enables highly accurate bi-allelic scoring of SNPs (single nucleotide polymorphisms) and indels (insertions/deletions) at specific loci across a wide range of genomic DNA samples. Bi-allelic discrimination is achieved through the competitive binding of two allele-specific forward primers, each with a unique tail sequence that corresponds with one of two universal probes; one labelled with FAM™ dye and the other with HEX™ dye.

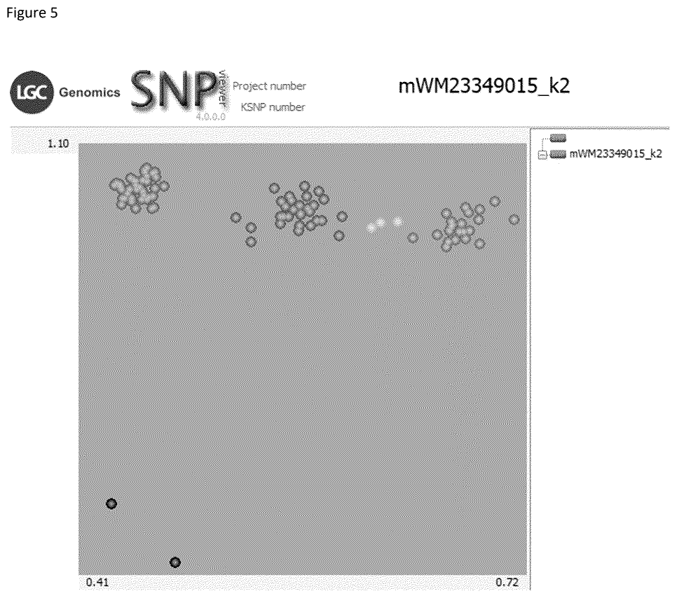

Apart from the DNA templates (genomic DNA of various watermelons lines or populations derived from crosses with Sidekick) and PCR-primers as described above, standard components (e.g. KASP-assay mix, KASP-master mix, etc.) and assay protocols were used in the assay as described by LGC, Biosearch Technologies™ world wide web at biosearchtech.com/products/pcr-kits-and-reagents/genotyping-assays/kasp-genotyping-chemistry). The Allelic Discrimination Plot (FIG. 5) differentiated between samples that were homozygous wild type, heterozygous and homozygous for the ClD14ins allele. It is noted that due to the presence of the repeat sequence/duplication in the ClD14ins allele, the ClD14ins allele containing samples (ClD14ins/ClD14 or ClD14ins-ClD14ins) generated more of signal compared to the wild type sample (ClD14-ClD14), whereby the distribution of the signal deviates from the classical distribution in the Allelic Discrimination Plot, but the genotypes are clearly distinguished in different clusters. The top left cluster in FIG. 5 is for plants homozygous for the wild type allele, the top right cluster is for plants homozygous for the mutant allele comprising the insertion/duplication, and the middle cluster is for heterozygous plants.

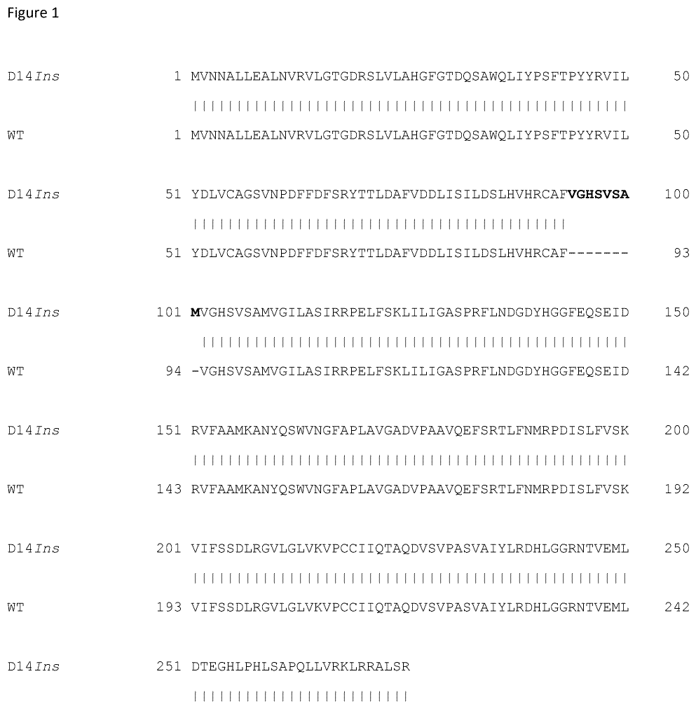

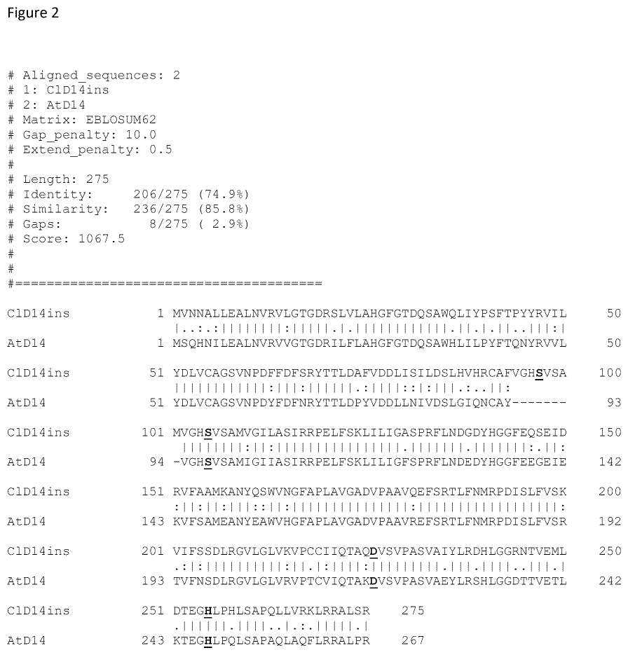

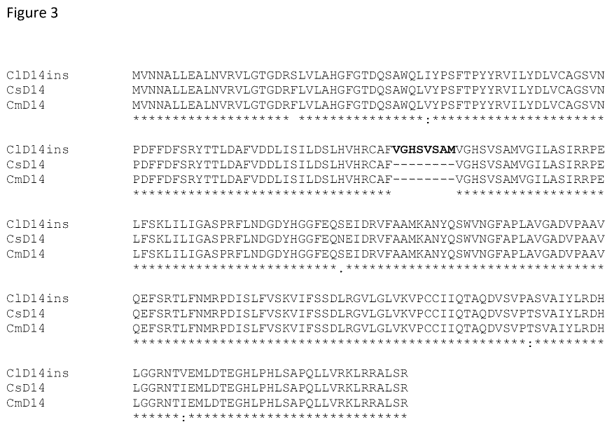

In FIG. 4 an alignment of the two genomic sequences (plus strand) is shown, with SEQ ID NO: 6 being the wild type genomic sequence (lacking the insertion) and SEQ ID NO: 5 being the mutant ClD14 sequence, comprising the insertion of 24 nucleotides, which in turn lead to an insertion (duplication) of 8 amino acids in the ClD14 protein, also referred to as ClD14ins (see FIGS. 1 and 3).

The above KASP assay can, thus, be used to detect the wild type ClD14 allele of SEQ ID NO: 6 (lacking an insertion/duplication of 24 nucleotides) or the mutant ClD14 allele of SEQ ID NO: 5 comprising an insertion (duplication) of 24 nucleotides in the genomic sequence. i.e. in SEQ ID NO: 5 nucleotides 280 to 303 are inserted, see FIG. 4, which is in fact a duplication of nucleotides 281 to 304 of the wild type sequence of SEQ ID NO: 6 (shown in italics in FIG. 4).

This KASP assay or other assays can be used to detect the wild type allele of the ClD14 gene of SEQ ID NO: 6, and/or a mutant allele of a ClD14 gene comprising one or more nucleotides inserted, deleted or replaced with respect to the wild type allele, such as for example the mutant allele of SEQ ID NO: 5.

BLAST analysis of the ClD14 protein against Uniprot/Swiss-prot was carried out to identify the orthologs of the ClD14 gene in other species, and two orthologs were identified, the Cucumis sativus CsD14 gene encoding the protein of SEQ ID NO: 8 and the Cucumis melo CmD14 gene encoding the protein of SEQ ID NO: 9.

### Example 2

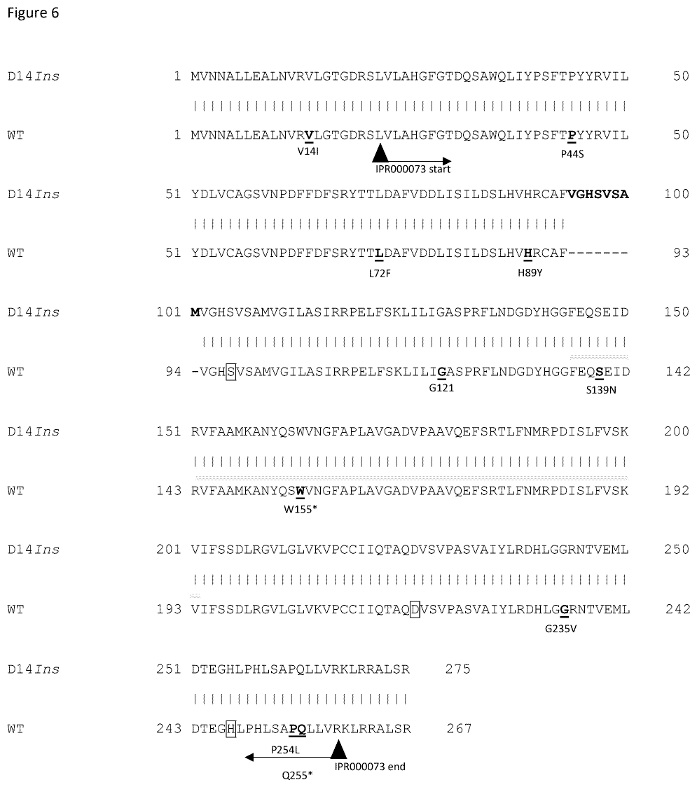

The watermelon TILLING population was screened, and several mutants were found in the ClD14 gene, leading to amino acid substitutions or STOP codons. The mutants are listed in Table 2 below and are also indicated in FIG. 6.

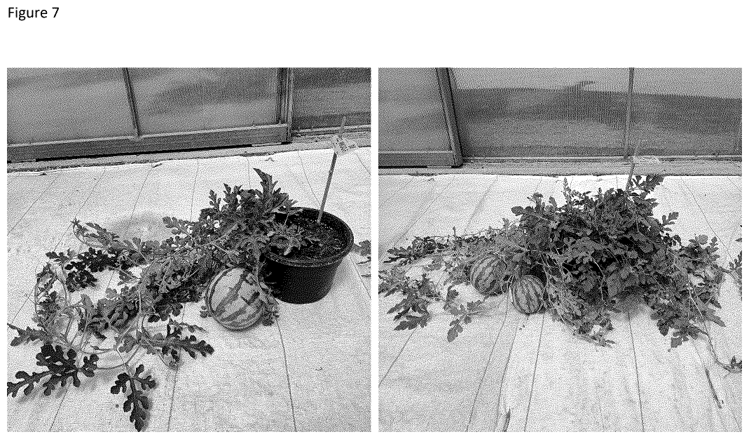

TABLE 2

| | | | Confirmed in plant | |
|---|---|---|---|---|
| | | | (He = | |
| | | | Heterozygous, Ho = | |
| Gene | Mutation in codon | Mutation | homozygous) | Phenotype |
| dwarf14 | GTC (V), ATC (I) | V14I | | |
| dwarf14 | CCT (P), TCT (S) | P44S | He | |
| dwarf14 | CTC (L), TTC (F) | L72F | | |
| dwarf14 | CAC (H), TAC (Y) | H89Y | | |
| dwarf14 | GGC (G), AGC (S) | G12IS | | |
| dwarf14 | AGC (S), AAC (N) | S139N | He | |
| dwarf14 | TGG (W), TAG (Stop) | W155Stop | He and | He = wild type |
| | | | Ho | Ho = |
| | | | | multibranching |
| | | | | see FIG. 7, |
| | | | | right photo |
| dwarf14 | GGC (G), GTC (V) | G235V | | |
| dwarf14 | CCT (P), CTT (L) | P254L | | |
| dwarf14 | CAG (Q), TAG (Stop) | Q255Stop | He | |

The W155Stop mutant has a multibranching phenotype, when the mutation is in homozygous form (W155*/W155*). The phenotype looks like that of the original mutant (ClD14ins/CID14ins), comprising the duplication of 8 amino acids. The average number of secondary branches was determined at 40 cm from the crown and is shown in Table 3, below.

TABLE 3

| | Average number | Relative increase in |
|---|---|---|
| | of secondary | secondary branches |
| | branches (40 cm | compared to wild type (set |
| Genotype of plants | from crown) | at 100% branches) |
| W155* allele | 31 | 238% |
| homozygous | | |
| W155* allele | 12 | |
| heterozygous | | |
| Wild type | 13 | 100% |

As the phenotypes are identical and as the W155* protein must be non-functional (it is truncated and lacks 113 amino acids of the C-terminal end), it can, surprisingly, be concluded that also the ClD14ins mutant allele must be encoding a non-functional protein, which does not transmit the signal to suppress secondary branching. Therefore, it is concluded that knock-out of the ClD14 gene or mutants that lead to non-functional ClD14 proteins, do not transmit any signal anymore, and, therefore, there is no inhibition of secondary branching anymore. This may also be referred to as “full multibranching” or “strong multibranching”.

This, now, also makes it clear that other mutants can be generated, whereby the multibranching phenotype is less strong, with some inhibition of secondary branching still being active, so that the multibranching phenotype is in-between the normal, wild type branching and the strong multibranching phenotype seen in ClD14ins/ClD14ins and in W155*/W155* plants, which results in about 240% of the average number of secondary branches relative to the wild type plants.

As the protein is highly conserved, with almost the entire protein being a conserved domain (IPR00073, see www at ebi.ac.uk/interpro/entry/InterPro/IPR000073/), single amino acid substitutions, deletions and/or insertions can be generated, e g. in the IPR00073 domain, which still enable some strigolactone binding to the protein pocket and some transmission of the signal in the strigolactone signalling pathway. For example, any of the TILLING mutants in in Table 2 above may, in homozygous form, lead to a reduced function ClD14 protein and to ‘intermediate multibranching’, e.g. at least about 110%, 120%, 130%, 140%, 150%, 160%, 170%, 180%, 190% secondary branches relative to the wild type plant, but not ‘full multibranching” as seen in plants where the ClD14 protein has lost its function and does not suppress secondary branching anymore.

Such more “moderate multibranching” or “intermediate multibranching” is desirable, as in the strong multibranching plants the humidity under the leaves gets high and diseases, such as fungi, easily develop.

Any newly induced mutants in the ClD14 gene (and plants comprising these in heterozygous or homozygous form), other than the already existing ClD14ins mutant (comprising an 8 amino acid duplication, shown in SEQ ID NO: 1), are, therefore, encompassed herein, especially mutants which result in a less strong multibranching (‘intermediate multibranching’) than the ClD14ins mutant or the W155* mutant.

However, also any knock-out mutant or mutant allele resulting in a non-functional ClD14 protein, other than the already existing ClD14ins mutant (comprising an 8 amino acid duplication, shown in SEQ ID NO: 1), is also encompassed herein, as are plants comprising such mutants in heterozygous or homozygous form.

### Example 3—Targeted Mutagenesis

Target-specific genome editing using engineered nucleases has become widespread in various fields. In watermelon Crispr has been successfully used to modify target genes, see e.g. Wang, Y., Wang, J., Guo, S. et al. CRISPR/Cas9-mediated mutagenesis of CIBG1 decreased seed size and promoted seed germination in watermelon. Hortic Res 8, 70 (2021). https://doi.org/10.1038/s41438-021-00506-1, which methods and vectors can also be used to generate mutations in the D14 gene.

Single-base substitutions or deletions of one or more nucleotides can be performed by homologous recombination (HR).

A binary CRISPR/Cas9 vector can be used, for example as described in Wang et al. (supra). Specific single guide RNAs (sgRNAs) targeted to D14 can be selected according to the assessment with CRISPR-P (http://cbi.hzau.edu.cn/crispr/). The target sequence is cloned into the vector is then used to transform a watermelon cultivar.

Watermelon explants can be transformed according to a modified method of Yu et al. (2011 Plant Cell Rep 30: 359-371). In brief, surface-sterilized watermelon seeds were sown on basic Murashige and Skoog solid medium supplemented with 3% Sue for 3 d. Then cotyledons without embryo were cut into 2×2 mm pieces. Agrobacterium tumefaciens strain EHA105 that harbors the vector can be used for transformation. The cotyledon explants are co-cultivated in the dark for 4 d and then transferred onto selective induction medium containing 1.5 mg/L 6 BA, 2 mg/L Basta. The regenerated adventitious buds are excised and transferred onto selective elongation medium, containing 0.1 mg/L 6 BA, 0.01 mg/L NAA, 2 mg/L Basta.

The plasmid vector harbors cassettes expressing CAS9 and two guideRNAs (gRNAs) and a donor fragment as template for homology-directed repair (HDR). Expression of the Cas9 gene and gRNA are driven by a strong promoter, such as a ubiquitin promoter. The gRNAs are be designed at opposite strands of the of the two targeting sites.

The donor fragment contains the desired mutation in the middle of a fragment that corresponds to the sequence of the target D14 gene (except for the mutation). Optionally, additional synonymous mutations, that do not change amino acid residues in the donor fragment, would prevent Cas9 from cutting the donor fragment again, once HDR is successfully achieved. The fragment is flanked with two gRNA target sequences including the PAM motifs, respectively, so that the donor DNA can be released by Cas9/gRNAs from the plasmid vector, see e.g. Sun et al. (2016) Molecular Plant 9, 62831 DOI: 10.1016/j.molp.2016.01.001.

To increase HDR, additional free DNA donor fragment can be co-introduced in the explant. After transformation, regenerated shoots selected based on e.g. plasmid vector encoded antibiotics resistance, are grown and analysed for the presence of mutations. This could be done by primers to amplify a target gene sequence from DNA by PCR. Primers are designed so that they cannot amplify a fragment from the plasmid. The amplified product can be sequenced to validate the presence of the mutation.

Plants can be regenerated from transformed plant material comprising the desired mutation using standard methods.

For example as described by Wang et al. (supra), genomic DNA can be extracted from young leaves of T0-T4 transgenic plants, which was then used for creating templates to amplify the specific fragments in the target gene using primers flanking two targeted sites. PCR can be conducted under the following conditions: 94° C./5 min; 94° C./30 s, 56° C./30 s, and 72° C./1 min (35 cycles); and 72° C./10 mm as the final extension. PCR products can directly be sequenced used standard methods.

The transgenic plants can also be verified as Cas9-free with primers specific for Cas9. PCR can be conducted under the following conditions: 94° C./5 min 94° C./30 s, 60° C./30 s, and 72° C./l min (29 cycles); and 72° C./10 min as the final extension.

1. A watermelon plant or plant part or a watermelon seed comprising a mutant allele of a gene named ClD14 (Citrullus lanatus Dwarf14), wherein the mutant allele encodes a protein which is truncated at the C-terminal end whereby at least one of the amino acids of the IPR00073 domain, starting at amino acid 22 and ending at amino acid 259 of SEQ ID NO: 2, is missing or is replaced by a different amino acid wherein the mutant allele results in said plant developing an increased average number of secondary branches when the mutant allele is in homozygous form,
wherein the ClD14 protein of the wild type allele is encoded by nucleic acid molecules selected from:
a) nucleic acid molecules, which encode a protein with the amino acid sequence given under SEQ ID NO: 2; or
b) nucleic acid molecules, which comprise the nucleotide sequence shown under SEQ ID NO: 6 or a complimentary sequence thereof.

2. The watermelon plant or plant part or a watermelon seed according to claim 1, wherein the mutant allele encodes a protein in which the codon for amino acid 155 or amino acid 255 is replaced by a stop codon.

3. The watermelon plant or seed according to claim 1, wherein the plant or seed is homozygous for the mutant allele and develops an increased average number of secondary branches compared to the plant which is homozygous for the wild type allele.

4. The watermelon plant, plant part or seed according to claim 1, wherein said plant, plant part or seed is diploid, triploid or tetraploid and said mutant allele is present in one or two copies in said diploid plant or plant part or seed or in two or four copies in said tetraploid plant or plant part or seed, or in one, two or three copies in said triploid plant or plant part or seed.

5. A method for detecting, and optionally selecting, a watermelon plant, seed or plant part comprising at least one copy of a mutant allele of a gene named ClD14 (Citrullus lanatus Dwarf14), comprising the steps of:
a) providing one or more genomic DNA samples of one or more watermelon plants, seeds or plant parts,
b) carrying out a genotyping assay, using the DNA samples of a) as template, that discriminates between the wild type ClD14 allele and the mutant ClD14 allele, wherein said genotyping assay is based on nucleic acid amplification making use of ClD14 allele-specific oligonucleotide primers, and/or wherein said genotyping assay is based on nucleic acid hybridization making use of ClD14 allele-specific oligonucleotide probes, and optionally
c) selecting a plant, seed or plant part comprising one or two copies of the mutant allele,
wherein the mutant ClD14 allele comprises one or more nucleotides inserted, duplicated, deleted or replaced with respect to the sequence of SEQ ID NO: 6, resulting in a mutant ClD14 protein which comprises one or more amino acids inserted, duplicated, deleted or replaced with respect to the sequence of SEQ ID NO: 2.

6. The method according to claim 5, wherein said ClD14 allele-specific oligonucleotide primers or said ClD14 allele-specific oligonucleotide probes comprise at least 10 nucleotides of SEQ ID NO: 6 or of the complement strand of SEQ ID NO: 6.

7. The method according to claim 5, wherein the mutant allele comprises at least one codon inserted or duplicated in the coding region of the allele, or at least one codon changed into another codon, or at least one codon deleted or changed into a STOP codon.

8. The method according to claim 5, wherein the mutant allele comprises the sequence of SEQ ID NO: 5.

9. The method according to claim 5, wherein the oligonucleotide primers or oligonucleotide probes comprise at least 15 nucleotides complementary to SEQ ID NO: 6 or to the complementary sequence of SEQ ID NO: 6.

10. The method according to claim 5, wherein said genotyping assay is a KASP-assay, said KASP-assay comprises a first forward primer detecting the wild type allele of SEQ ID NO: 6 in the DNA sample, a second forward primer detecting the mutant allele comprising one or more nucleotides inserted, deleted or replaced with respect to SEQ ID NO: 6 in the DNA sample, and one common reverse primer.

11. A method for generating a PCR amplification product and/or an oligonucleotide hybridization product of a part of the genomic DNA of watermelon plants, seeds or plant parts comprising the steps of:
a) providing a sample or a plurality of samples of genomic DNA of a watermelon plant or of a plurality of plants,
b) providing at least one pair of PCR primers or at least one oligonucleotide probe, which primers or oligonucleotide probe comprise at least 10, 11, 12, 13, 14, 15, 16, 17, 18, 19, 20, 21, 22 or more consecutive nucleotides of the genomic allele of the ClD14 gene and can hybridize to the genomic allele and/or amplify part of the genomic allele in a PCR assay, and
c) carrying out a PCR assay using the primer pair or a hybridization assay using the probe of step b) on the sample(s) of step a) to generate a PCR amplification product and/or an oligonucleotide hybridization product, and optionally
d) selecting a plant or plant part or seed comprising one or two copies of an allele of the ClD14 gene in the genome,
wherein the wild type allele of the ClD14 gene encodes the protein of SEQ ID NO: 2 or comprises the genomic DNA of SEQ ID NO: 6 or the complementary sequence of SEQ ID NO: 6.

12. A method for amplifying and/or hybridizing a part of the genomic DNA of watermelon plants, seeds or plant parts comprising the steps of:
a) providing a sample or a plurality of samples of genomic DNA of a watermelon plant or of a plurality of plants,
b) providing at least a pair of PCR primers or at least one oligonucleotide probe, which primers or oligonucleotide probe comprise at least 10, 11, 12, 13, 14, 15, 16, 17, 18, 19, 20, 21, 22 or more consecutive nucleotides of the genomic allele of the ClD14 gene and can hybridize to the genomic allele and/or amplify part of the genomic allele in a PCR assay, and
c) carrying out a PCR assay using the primer pair or a hybridization assay using the probe of step b) on the sample(s) of step a) to generate a PCR amplification product and/or an oligonucleotide hybridization product, and optionally
d) selecting a plant or plant part or seed comprising one or two copies of an allele of the ClD14 gene in the genome,
wherein the wild type allele of the ClD14 gene encodes the protein of SEQ ID NO: 2 or comprises the genomic DNA of SEQ ID NO: 6 or the complementary sequence of SEQ ID NO: 6.

13. The method according to claim 12, wherein said plurality of plants in step a) is selected from: an F2 population, inbred lines, a backcross population, a breeding population, or hybrid plants.

14. The method according to claim 12, wherein said one or two copies of an allele of the ClD14 gene in step d) are selected from a wild type allele and/or a mutant allele of the ClD14 gene.
